(19) United States
(12) Patent Application Publication
(10) Pub. No.: US 2016/0137975 A1
(43) Pub. Date: May 19, 2016

(54) GENERATION OF MALE GERM CELLS

(71) Applicant: UNIVERSITY OF PITTSBURGH - OF THE COMMONWEALTH SYSTEM OF HIGHER EDUCATION, Pittsbugh, PA (US)

(72) Inventors: Cyril Yeshwant Ramathal, San Jose, CA (US); Jens Dumuthy-Durruthy, Stanford, CA (US); Renee A. Reijo Pera, Los Altos, CA (US); Kyle E. Orwig, Pittsburgh, PA (US)

(21) Appl. No.: 14/904,396

(22) PCT Filed: Jul. 10, 2014
(86) PCT No.: PCT/US14/46200

§ 371 Date: Jan. 11, 2016

Related U.S. Application Data
(60) Provisional application No. 61/844,802, filed on Jul. 10, 2013.

(51) Int. Cl. C12N 5/076; A61K 9/00; A61K 35/52; C12N 5/074; A61K 35/545

(52) U.S. Cl. CPC C12N 5/061; C12N 5/0696; A61K 35/545; A61K 35/52; A61K 9/0034; C12N 2506/45; C12N 2506/02; C12N 2501/603; C12N 2501/602; C12N 2501/604; C12N 2501/606; C12N 2501/605; C12N 2501/608

(57) ABSTRACT

Methods are provided for the generation of male germ cells from somatic cells. Included are methods of non-integrative reprogramming for germ cell differentiation with a reduced risk of neoplasia during in vivo differentiation by the inclusion of VASA with the reprogramming factors. Also included are methods of generating male germ cells from reprogrammed pluripotent cells by direct injection of the reprogrammed cells into the seminiferous tubules. In some embodiments the somatic cells are derived from a male with oligospermia or azoospermia, which may be the result of a genetic abnormality in Azoospermia Factor (AZF) region.

### GOVERNMENT RIGHTS

This invention was made with Government support under contract HD068158 awarded by the National Institutes of Health. The Government has certain rights in this invention.

### BACKGROUND OF THE INVENTION

Germ cells are the cells that give rise to the gametes of a sexually reproductive organism. They are the only cells that can undergo meiosis as well as mitosis. During early development, primordial germ cells (PGCs) are segregated from the lineages of somatic cells, and migrate to the developing gonads. Proliferation also occurs during migration.

The local cellular environment around germ cells influences their development. In males, the Sry gene induces somatic cells to develop into a testis, which tissue includes Sertoli cells. Sertoli cells stimulate PGCs to differentiate toward sperm. In human males, spermatogenesis begins at puberty in seminiferous tubules in the testicles and goes on continuously. Spermatogonia proliferate continuously by mitotic divisions around the outer edge of the seminiferous tubules, next to the basal lamina. Some of these cells stop proliferation and differentiate into primary spermatocytes. After they proceed through the first meiotic division, two secondary spermatocytes are produced. The two secondary spermatocytes undergo the second meiotic division to form four haploid spermatids. These spermatids differentiate morphologically into sperm by nuclear condensation, ejection of the cytoplasm and formation of the acrosome and flagellum.

Infertility refers to failure of a couple to conceive following a period of unprotected regular intercourse; estimates for prevalence of the condition range from 10-25% of couples in the United States. Male factor infertility is partially or fully responsible for approximately 30-55% of cases of infertility. Azoospermia, which is the complete absence of sperm in the ejaculate, accounts for 10-15% of male infertility cases and generally affects 1% of the male population. Azoospermia is divided into two major categories: obstructive azoospermia (OA), in which there is genital tract outflow obstruction, blocking passage of the sperm, and non-obstructive azoospermia (NOA), in which the testicle fails to produce mature sperm in the ejaculate. Azoospermia of a genetic origin is primarily caused by a wide array of genetic disorders, such as chromosomal abnormalities, monogenic disorders, multifactorial genetic diseases, and epigenetic disorders.

There is great clinical interest in methods of generating male germ cells for therapeutic purposes, including treatment of low sperm counts or azoospermia. The present invention addresses this need.

### SUMMARY OF THE INVENTION

Methods are provided for producing male germ cells from somatic cells, including without limitation fibroblasts. In the methods of the invention, a somatic cell reprogrammed to pluripotency is induced to differentiate into a male germ cell by introducing the pluripotent cell into a Sertoli cell environment. In some embodiments the introducing step comprises the step of directly injecting the pluripotent cell into the seminiferous tubules of a male mammal, wherein the reprogrammed cell differentiates to generate male germ cells. In some embodiments the reprogrammed pluripotential cells are transplanted in the absence of in vitro differentiation along the germ cell pathway, i.e. the transplanted cells are pluripotent. In some embodiments the germ cells give rise to spermatogonia in the in vivo setting.

In some embodiments, somatic cells are reprogrammed to pluripotency by a non-integrative method, e.g. by the introduction into the cell of a cocktail of modified mRNA encoding reprogramming factors; by the introduction into the cell a cocktail of proteins that are reprogramming factors; and the like. Preferably the cocktail of factors includes VASA (also known as Ddx4) protein, or an mRNA encoding VASA. In some embodiments the cocktail of reprogramming factors comprises OCT3/4 [O], SOX2 [S], KLF4 [K], cMYC [M] and VASA [V]. Transient ectopic expression of OSKMV reprograms human fibroblasts into a stable pluripotent state, which reprogrammed cells have a higher efficiency of germ cell formation in vivo in the testis relative to cells reprogrammed without VASA, and further have been shown to be non-tumorigenic in an animal model.

In some embodiments the somatic cells are derived from a human male with oligospermia or azoospermia, which may be non-obstructive azoospermia (NOA). In some such embodiments, the azoospermia results from a genetic abnormality, for example a deletion, in the (AZF) region. Such genetic abnormalities may include a deletion in one or more of AZFa, AZFb and AZFc. Surprisingly, it is found that even azoospermic somatic cell donors can give rise to germ cells through the process of somatic cell reprogramming and direct transplantation into the seminiferous tubules.

In some embodiments a treatment is provided for male infertility associated with azoospermia resulting from a genetic abnormality, for example a deletion, in the (AZF) region. Such genetic abnormalities may include a deletion in one or more of AZFa, AZFb and AZFc. In such methods, a somatic cell is obtained from a male donor suffering from infertility. The somatic cell is reprogrammed to pluripotency, optionally with a cocktail of reprogramming factors in combination with VASA. The pluripotent cells are injected directly into the seminiferous tubules of the donor, where the pluripotent cells are brought into contact with a Sertoli cell environment and induced to differentiate into male germ cells, including sperm cells. Additional mRNAs or genes may be introduced to supplement deleted genes. For example, men with DAZ gene deletions may be provided DAZ mRNA in the reprogramming mixture or prior to introduction.

These and other objects, advantages, and features of the invention will become apparent to those persons skilled in the art upon reading the details of the subject methods and compositions as more fully described below.

### BRIEF DESCRIPTION OF THE DRAWINGS

The invention is best understood from the following detailed description when read in conjunction with the accompanying drawings.

FIG. 1. Functional validation of mRNA expression encoding for VASA. Immunostaining of VASA in mRNA transfected BJ fibroblasts 24 h after transfection. VASA protein localized correctly in the cytoplasm. Mock transfected, secondary antibody stained only and non-transfected samples served as negative controls. Scale bar, 10 μm.

FIG. 2. Functional and molecular studies of RiPSC.HUF1 derived with OSKM and OSKMV. (a) Gene expression analysis of markers associated with the germ line lineage in an undifferentiated state. Three genes (PRDM14, VASA, DPPA3) showed significantly higher expression in OSKMV derived clones (purple bar) compared to OSKM. (student t-test, mean±s.d.; n≧16 for each gene and sample *p<0.05, **p<0.01, ***p<0.001, ****p<0.0001). (b) Gene expression analysis of clones derived with OSKM and OSKMV, RiPSC.BJ line, and H9 during in vitro differentiation into primordial germ cells with BMP4. Samples were isolated at day 0, 2, and 4 post BMP4/vehicle treatment. Three key markers (NANOS3, VASA, DPPA3) were upregulated in OSKMV derived clones (student t-test, mean±s.d.; n≧16 for each gene and sample *p<0.05, **p<0.01, ***p<0.001, ****p<0.0001). (c) Quantification of meiotic spreads. Approximately 200-300 cells were counted for each line. Staining patterns were classified as negative, punctuated or elongated. p=0.0299 for elongated and p=0.0077 for punctuated staining (student t-test, mean±s.d.; n≧200 for each sample).

FIG. 3. Transplantation of OSKM and OSKMV cells into busulfan-treated mouse testes. (a) Histology cross-sections of tubules inside mouse testes stained with co-localizing human-specific NUMA (red) and VASA (green). White arrows indicate transplanted cells positive for VASA germ cell marker; red dashed arrows indicate VASA negative transplanted iPSCs. Scale bar, 50 μm. (b) Histology cross-sections of tubules of mouse testes stained with additional germ cell specific markers (DAZ, PLZF) demonstrating NUMA co-localization. H9 cells are negative for germ cell marker selection. White arrows indicate transplanted cells positive for VASA germ cell marker; red dashed arrows indicate VASA negative transplanted iPSCs. Scale bar, 50 μm. (c) Weight of testis two months after transplantation of cells (OSKM and OSKMV cells with two controls, Human Testis and H9 hESC line). (d) Histology cross-sections of entire mouse testis with cell masses after OSKM cell transplantation. Testes were harvested two months after cell transplantation. Non-treated mouse testis is shown as negative control. Scale bar, 300 μm.

FIG. 4. Transplantation of OSKM and OSKMV followed by quantification analysis. (a) Hematoxylin and eosin staining of histology cross-sections of tubules inside mouse testes. Black arrows indicate individual tubules. Yellow arrows indicate cartilage formation in H9 transplanted cells. Scale bar, 100 μm. (b) Representative low magnification image of histology cross-section of multiple tubules stained with NUMA and VASA indicating that only a fraction of tubules formed VASA positive cells. Red dashed rectangle indicate tubules with NUMA only cells along the basement membrane. White rectangle indicate NUMA/VASA positive tubules. Scale bar, 50 μm. (c) Representative image of histology cross-section of one single tubule. Single NUMA/VASA positive cells were counted for quantification. Scale bar, 50 μm. (d-f) Quantification of immunohistochemistry results of cross-sections from OSKM and OSKMV cells compared to both controls. All serial sections were subject to counting (see Materials and Methods) (d): Percentage of tubules with positive VASA/NUMA co-staining calculated (against total number of counted tubules). (e): For each positive tubule, VASA positive cells that co-stained with NUMA were counted and calculated against positively stained tubules. (f): Relative germ cell forming potential calculated by multiplying fraction of positively stained tubules with number of VASA/NUMA co-stained cells for each sample. Scale bar, 80 μm.

FIG. 5. Germ cell formation of OSKM and OSKMV transplanted cells in vivo. (a) Whole mount analysis on transplanted human fetal testis cells and OSKMV cells into mouse testes. Chain (white dashed rectangle) and cluster formation (white arrows) visible in human fetal testis control cells. Transplanted OSKMV cells gave rise to cluster formation indicated by white dashed rectangle. Scale bar, 50 μm. (b) Observed endogenous germ cell activity inside the basement membrane of transplanted mouse testes. Cells stained positive for VASA, PLZF and DAZ but do not co-localize with NUMA indicating residual germ cells after busulfan treatment. Scale bar, 50 μm. (c) Detection of Sertoli cells indicated by GATA4 staining that is of mouse origin and near OSKMV transplanted cells at the basement membrane of mouse testes. Scale bar, 50 μm. (d) Additional germ cell markers stain positive with NUMA in OSKM and OSKMV transplanted cells in mouse testes and co-localize with each other. GFRα1 was only detected in OSKMV transplanted cells and human fetal testis control sections. Yellow dashed rectangles indicate magnified snapshots in each panel and for each sample. Scale bar, 80 μm.

FIG. 6. Validation of in vitro transcribed mRNA encoding for VASA. (a) Quantitative measurement of ivT product before (1100 ng/μl) and after dilution to working concentration (100 ng/μl). Ratio of A260/A280 indicates pure product. (b) Denaturing formaldehyde agarose gel for size validation. Synthesized VASA mRNA had the correct size and little degradation was observed.

FIG. 7. Derivation of mRNA induced pluripotent stem cells in feeder- and xeno-free conditions. (a) Flowchart of cloning strategy. First, a DNA vector containing “backbone sequence” was designed. PCR amplification of the ORF of interest was performed with a specific forward primer (containing the Kozak sequence and restriction enzyme X sequence) and a specific reverse primer (containing the restriction enzyme Y sequence). Both, backbone clone and amplified ORF were digested with restriction enzyme X and Y and ligated. Next, transformation of template clone and amplification of positive clones was carried out, followed by a final digest with restriction enzyme Z to cut out ORF+UTR. A polyA tail was added at the 3′ end with a Tail PCR and this template was subject to in vitro transcription reaction. (b) Backbone sequence and DNA template cloning strategy for in vitro transcription of modified RNA. Sequence contains all essential features such as UTR regions, a multiple cloning site (MCS), a T7 promoter and specific restriction enzyme sites. (c) Detailed sequence of in vitro transcribed template encoding a gene of interest. Arrows indicate restriction enzyme cleavage sites. +1 indicates the first base incorporated into RNA during transcription. SpeI=restriction enzyme Z, NheI and AgeI=restriction enzyme X and Y, respectively. (d) Overview of feeder-free reprogramming with modified mRNA. (e) Morphology tracking of reprogrammed human fibroblasts during the course of 8 days. Fibroblasts show early epitheliod morphology (day 4), small cluster formation that form into small hES cell like colonies (day 4-7). Small colonies grow in size and become mature RiPSC colonies (day 8). Arrow indicates forming colony. Mock transfected cells proliferated until 100% confluent by day 8. (f) Live staining against Tra-1-60 and Tra-1-81 during reprogramming for colony identification. Scale bar, 150 μm. Abbreviations: HBA, hemoglobin alpha; IVT, in vitro transcription; ORF, open reading frame; PCR, polymerase chain reaction; UTR, untranslated region.

FIG. 8. Pluripotency assessment of lentiviral/mRNA derived iPS cell lines (HUF1, BJ) with OSKM and OSKMV. (a) No differences in morphology, AP staining nor the ability to form embryoid bodies detected in OSKM and OSKMV derived clones. (b) Gene expression of endogenous VASA was examined by immunocytochemistry in undifferentiated OSKMV cells. Scale bar, 15 μm. (c) Gene expression of markers associated with pluripotency in lentiviral/mRNA derived HUF1 and BJ iPSCs and in lentiviral HUF1 iPSCs. A great subset of markers were significantly decreased in OSKMV derived clones compared to OSKM. (student t-test, mean±s.d.; n≧16 for each gene and sample *p<0.05, **p<0.01, ***p<0.001). (d) Gene expression of markers associated with germ cell lineage in RiPSC.BJ and lentiviral derived HUF1 iPSCs. Ectopic lentiviral VASA expression was confirmed in OSKMV cells. (student t-test, mean±s.d.; n≧16 for each gene and sample *p<0.05). (e) Immunostaining showing expression of all three lineage markers after in vitro differentiation of RiPSC.HUF1 cells derived with OSKM and OSKMV. (f) Bisulfite sequencing of H9, HUF1 fibroblast and RiPSC.HUF1 derived with OSKM and OSKMV. (g) Normal karyotype of RiSPC.HUF1.OSKM line. (h) Derived teratomas showing ectoderm (neural rosettes, epidermis), mesoderm (cartilage), and endoderm (gut-like endothelium) of RiPSC.HUF1 cells derived with OSKM and OSKMV.

FIG. 9. Molecular and functional characterization of HUF1 RiPSCs derived with OSKM and OSKMV. (a) Morphological changes of RiPSC.HUF1 clones upon BMP4 differentiation. (b) Gene expression analysis of pluripotency associated genes during PGC differentiation. (student t-test, mean±s.d.; n≧16 for each gene and sample *p<0.05, **p<0.01, ***p<0.001) (c) Qualitative analysis of meiotic progression. Meiotic spreads were prepared for all samples followed by immunostaining against the meiotic specific SCP3 (green), CENPA (red) and counterstained with DAPI. Each image is representative. Staining patterns were classified as negative, punctuated or elongated. Scale bar, 10 μm. (d-e) Differential methylation analysis at four imprinted genes with bisulfite sequencing. d: Quantitative calculation of bisulfite sequencing results. Percentage of methylated CpG islands was calculated for each sample and imprinted gene (Fisher's exact test, mean±s.e.m.; n≧14 for each gene and sample, ****p<0.0001). e: Bisulfite sequencing of four loci in five different samples. Unique sequences of each DNA clone are represented as rows of circles, with each circle symbolizing the methylation state of one CpG (black=methylated, white=demethylated).

FIG. 10. RNAseq to assess global gene expression of OSKM and OSKMV cells. (a-d) Pairwise comparison between all four samples using scatter plots, volcano plots, JS distance, and significant genes overview matrix. (e-f) Principal component analysis (PCA) and multidimensional scaling (MDS). (g-h) Hierarchical clustering and heatmap of differential expressed genes of RNAseq data.

FIG. 11. Transplantation of undifferentiated iPSCs into mouse testes. (a) Immunohistochemistry of Human Fetal Testis and Mouse tissue cross-sections. NUMA antibody specifically recognizes human cells (nuclear). Scale bar, 30 μm. (b) IgG control for NUMA primary antibody. Scale bar, 50 μm. (c) Immunohistochemical analysis of cross section. Transplanted OSKMV cells into mouse testes were stained for germ cell differentiation events. Co-localization of VASA and DAZ, counterstained with DAPI. Scale bar, 50 μm. (d) Representative images of OCT3/4 stained cross sections of iPSC transplanted mouse testes. OCT3/4 co-localized with NUMA intra- and extra-tubular in an unorganized fashion. Specific OCT3/4 staining along the inside of the basement membrane of the mouse tubule appeared to be of mouse origin and did not co-localize with NUMA. Scale bar, 50 μm.

FIG. 12. Derivation and characterization of iPSCs from azoospermic patient fibroblasts. (A) Genotype-phenotype map to visualize deletion of AZF a, b and c regions in hESC lines and patient samples used for this study. A deletion map was constructed for every hESC line, patient fibroblast and iPSC line by testing for the presence of 20 major Sequence-Tagged-Sites (STS) sites by PCR. Vertical black bars (top) represent the STS amplification sites and genes we used to diagnose AZFa, b and c deletions. Dashed lines indicate the absence of a Y chromosome in female H9 hESCS. Grey boxes represent the deleted regions of the Y chromosome. The fertility phenotype (SCO=Sertoli cell only; EMA=early maturation arrest) of each patient is listed (left) and karyotype (right). The Δ symbol indicates the deletion of an AZF region in that cell line. (B) Immunocytochemistry in all patient-derived iPSC lines for nuclear (OCT4, NANOG) and cell surface (SSEA1/3/4, TRA1-60, TRA1-81) markers of pluripotency. (C) In vitro differentiation of 4 patient-derived lines to cells representative of all three germ layers (AFP=α-fetoprotein, βIII-Tub=β-III-tubulin/Tuj1, SMA=smooth muscle actin). (D) In vivo teratoma formation of iAZFΔbc, iAZFΔc & iAZFΔa lines showing evidence of all 3 germ layers (gut-like endoderm, muscle-like mesoderm and neural ectoderm).

FIG. 13. Characterization of iPSC-derived germ cell differentiation and germ cell identity in vitro. (A) Efficiency of germ cell differentiation of AZF-intact, control iPSCs (iAZF1), hESCs (H1) and AZF-deleted iPSCs (iAZFΔc, iAZFΔbc, iAZFΔa). Percentage of VASA:GFP+ populations obtained from FACS sorting of two independent clones of each cell line after 7 days of differentiation. (B) Immunocytochemistry of GFP+ cells for GFP protein (red) and VASA protein (green). Nuclei are counterstained with DAPI (blue). Scale bar, 50 μm. (C) Heat maps of single cell RT-PCR measurements (20 single cells; rows in heat map) and 27 germ cell genes (columns in heat map). Primordial germ cell markers and Post-primordial germ cell (Post-PGC) markers are indicated at top. Legend of heat index indicates high (red) and low (blue) gene expression; grey=not present. (D) The percentage of GFP+ sorted single cells expressing GFP transcript and 12 additional germ cell-associated genes as plotted by the number of markers within each individual cell. Dashed, vertical lines indicate the segment of the population of single cells from each line relative to the middle 70-80% of control, iAZF1 population. Error bars indicate SEM (n=2); asterisk, significant difference in % of VASA:GFP+ population by two-tailed students t test (p<0.05).

FIG. 14. Germ cell differentiation of hESCs engrafted in mouse spermatogonial tubules. Testis xenografts were analyzed by immunohistochemistry using a human cell-specific antibody, NUMA and a pan-germ cell marker, VASA antibody. (A) Human fetal testis (22 week-old) with nuclear localization of NUMA (red) and cytoplasmic VASA expression (green) in germ cells. Xenografts derived from (B) H1 hESC (XY) and (C) H9 hESC (XX). In all images: single channels for NUMA (i; red) and VASA (ii; green) are shown for a specific region. White rectangles indicate regions of positive co-localization of NUMA and VASA (iii & iv) and represented as merged channels (MERGE; NUMA+VASA+DAPI). Arrows indicate cells or clusters with positive co-localization of NUMA and VASA. Asterisks (*) indicate NUMA-negative, endogenous mouse germ cells. Dotted white lines indicate the outer edges of spermatogonial tubules. Sections were counterstained with DAPI (blue). Scale bar, 50 μm.

FIG. 15. Germ cell differentiation of patient-derived iPSCs engrafted in mouse spermatogonial tubules. Testis xenografts were analyzed by immunohistochemistry using a human cell-specific antibody, NUMA and a pan-germ cell marker, VASA antibody. Xenografts derived from (A) iAZF1; (B) iAZFΔc; (C) iAZFΔbc & (D) iAZFΔa cell lines. In all images: single channels for NUMA (i; red) and VASA (ii; green) are shown for a specific region. Nuclei are counter-stained with DAPI (blue). Regions of positive co-localization of NUMA and VASA (iii-v) are represented as merged channels (MERGE; NUMA+VASA+DAPI). Arrows indicate cells or clusters with positive co-localization of NUMA and VASA. Dashed white lines indicate the outer edges of spermatogonial tubules. Scale bar, 50 μm. (E) Percentage of tubules with positive VASA/NUMA co-staining were calculated (against total number of counted tubules). (F) For each positive tubule, the fraction of VASA/NUMA double-positive cells was determined. (G) The relative germ cell potential for human fetal germ cells and iPSC lines as a product of % tubule occupancy and number of NUMA/VASA-costained cells per tubule. Data are represented as mean+/−SEM of replicates. Significant differences in percentages/ratios were determined by one-way analysis of variance (ANOVA). *, P<0.05; **, P<0.001.

FIG. 16. Xenotransplanted iPSCs and hESCs exhibit multiple markers of PGCs and gonocytes. Xenografts of H1, iAZF1 and AZF-deleted human iPSCs were stained in adjacent, serial cross sections for the germ cell markers VASA, DAZL, UTF1, STELLA, PLZF and DAZ in NUMA+ regions. (A) Cross-section of a human fetal testis (22 wks) with positive immunostaining for all markers tested. Insets in each panel indicate zoomed-in regions. Scale bar, 50 μm. (B-F), Cross-sections of mouse testes after transplantation of iAZF1, H1 hESC, iAZFΔc, iAZFΔbc and iAZFΔa cell lines respectively. For each triplet of panels, NUMA+VASA double positive cells are shown adjacent to each marker without and with DAPI counterstaining. Immunostaining for STELLA is shown in the first column for each iPSC line, while all other markers are shown horizontally and labeled with the marker being tested. Dashed white lines indicate the outer edges of spermatogonial tubules. Nuclei are counterstained with DAPI (blue). Scale bar, 30 μm.

FIG. 17. DNA methylation properties and Meiotic activity in Xenotransplanted iPSCs. Human fetal (22 wk) and adult testes and testis xenografts of iAZF1 human iPSCs were stained for VASA, 5-methylcysotine (5 MC), 5-hydroxymethlcytosine (5 hMC), Synaptonemal Complex protein-3 (SCP3) and phosphorylated histone H2AX (γH2AX) in NUMA+ regions. (A) Cross-section of a human fetal testis (22 wks) with positive immunostaining for VASA, 5 MC and 5 hMC. Areas in white rectangles are shown in higher magnification below each panel. (B) Cross-sections of mouse testes xenografts after transplantation of undifferentiated iAZF1 cells with immunostaining for VASA, 5 MC and 5 hMC. Non-germ cell containing regions of the xenografts are shown in right column. Scale bar, 50 μm. Cross-section of a human fetal testis (C) and human adult testis (D) with positive immunostaining for SCP3, VASA and γH2AX. Areas in white rectangles are shown in higher magnification below each panel. (E) Cross-sections of mouse testes after transplantation of undifferentiated iAZF1 cells with immunostaining for NUMA, VASA, 5 MC and 5 hMC. Non-germ cell containing regions of the xenografts are shown in right column. Scale bar, 50 μm. In all panels, dashed lines indicates edges of spermatogonial tubules. Dotted ovals indicate cells with differential 5 MC and 5 hMC signals.

FIG. 18. Schematic illustration of major findings of this study. A schematic summarizing the major findings of this study. iPSC derived from AZF-intact and AZF-deleted patients form PGCS in vitro with reduced germ cell ‘identity’ and limited meiotic entry in the latter. When transplanted, male hESCs and iPSCs specifically differentiate to PGCs and gonocytes inside the spermatogonial tubule niche where they contact sertoli cells. However, outside the tubule, all patient-derived iPSCs and hESCs remain undifferentiated as primitive tumors. The transplantation strategies proposed here offer a potential avenue for fertility restoration for infertile men.

FIG. 19. Generation and characterization of iPSCs from normal and azoospermic patients. (A) Excision of the STEMCCA reprogramming cassette in iPSC clones from 4 patient-derived fibroblasts. PCR was performed with primers against 3′ end of STEMCCA cassette and excised clones were confirmed by the absence of an amplified fragment (arrows). The original donor plasmid (STEMCCA) and pre-excised iPSC (iAZFΔbc, iAZFΔc) were used as positive controls while the donor fibroblasts (AZFΔbc Fibr, AZFΔc Fibr) were used as negative controls. GAPDH expression was measured by PCR as a loading control. (B) Heat map representation of Fluidigm gene expression analysis of 10 core pluripotency-associated genes in H9 hESC cells, donor fibroblasts, pre- and post-excised clones (red=high expression, blue=low expression). (C) Spectral karyotyping (SKY) analysis of iPSC lines. (D) Summary table of all patient lines derived with corresponding AZF regions deleted, karyotype, clinical spermatogenesis phenotype and iPS derivation method.

FIG. 20. Single cell hierarchical clustering of VASA:GFP sorted populations. (A) IPSCs from AZF-intact (iAZF1) and AFZ-deleted cells (iAZFΔc, iAZFΔbc & iAZFΔa) were transduced with VASA:GFP lentivirus, differentiated for 7 days and sorted for GFP+ populations. Single cell gene expression was analyzed by Fluidigm-based qRT-PCR. 150-160 single cells were analyzed and shown above for each cell line. Normalized gene expression values were hierarchically clustered by average linkage method for all germ cell marker genes in a heat map. (B) Linear discrimination analysis was performed on single cells (n=150-160) of each population as indicated in the legend. Each data point represents a single cell of each population.

FIG. 21. Meiotic activity of germ cells derived in vitro from iPSC lines. (A) Synaptonemal Complex Protein-3 (SCP3) expression in VASA:GFP+ populations of each cell line. Punctate SCP3 (top) staining indicates pre-meiotic cells while elongated, fiber-like SCP3 staining (bottom) labels cells entering meiosis I (leptotene, zygotene or pachytene stages). Phosphorylated histone H2AX (γ-H2AX), a marker of DNA strand breaks during meiosis I was used as an additional marker. As controls, spermatocytes isolated from a mouse testis were also stained for SCP3 (left column). (B and C) Quantification of unstained, pre-meiotic and meiotic cells by SCP3 staining localization in at least 1000 cells of each cell line tested (B) and of only SCP3-positive cells (C); Scale bar, 50 μm. (D) In two independent experiments, germ cells derived from iPSCs on D7 of differentiation were stained with propidium iodide and analyzed by FACs for haploid (1N), diploid (2N) and tetraploid (4N) cells to evaluate the percentage of putative haploid germ cells. As a control, human sperm were also stained and verified to be largely 1N cells (top); Scale bar, 25 μm. Haploid cells were sorted and subjected to (E) DNA Fluorescent In Situ Hybridization (DNA FISH) for chromosomes 16 and 18 to verify single chromosome copies Scale bar, 25 μm. (F) Genomic DNA from VASA-GFP+ cells from AZF-intact (iAZF1) and AFZ-deleted cells (iAZFΔc, iAZFΔbc & iAZFΔa) was bisulfite-converted and PCR amplification of imprinted loci was carried out with specific primers annealing to the PEG1 or H19 DMR regions respectively. Analysis for CpG methylation was performed using the BiQ Analyzer (V3.0) software. 20 bacterial clones were analyzed per population. The fraction of methylated CpGs was determined for PEG1 and H19 DMRs from all clones per cell line. Dashed grey line indicates 50% methylation. As a control, human sperm DNA was also analyzed and quantified here. Error bars indicate SD of replicate clones (n=30 for PEG1; n=18 for H19).

FIG. 22. Survival of iPSC and hESC post-transplantation in mouse spermatogonial tubules. (A) Human fetal testis cells, (B) H1 human ES cells, (C) AZF-intact or (D-F) AZF-deleted human iPSCs were injected into seminiferous tubules of busulfan-treated mouse testes (n=8). After 2 months, xenografts were analyzed by performing whole-mount immunohistochemistry using an anti-human antibody. Representative images for spermatogonial chain/cluster formation (green) (i) and magnified cells are shown (ii & iii). Arrows indicate A_{single}-like cells or chains of cells in each image. Arrowheads (ii & iii) indicate surviving human donor cells in singlets, pairs or clusters. Wherever shown, dashed lines indicate edges of spermatogonial tubules. For all cell lines, at least 3 testes were analyzed by whole mount immunohistochemistry and representative images shown. Scale bar, 100 μm. Xenografts were also analyzed by immunohistochemistry using NUMA & VASA antibodies. Nuclear localization of NUMA (red) and cytoplasmic VASA expression (green) in donor-derived cells. Xenografts derived from (G) iAZF1; (H) H1 hESC; (I) iAZFΔc; (J) iAZFΔbc & (K) iAZFΔa cell lines. In all images: merged channels for NUMA (red) and VASA (green) co-localized in the same cells inside spermatogonial tubules. Asterisks indicate spermatogonial tubules that are not filled with NUMA/VASA double-positive cells. Sections were counterstained with DAPI (blue). Scale bar, 100 μm.

FIG. 23. Transplanted iPSCs and hESCs did not form GATA4-positive cells and express OCT4 and SOX2. Xenografts of H1, iAZF1 and AZF-deleted human iPSCs were stained in adjacent, serial cross sections for early germ cell markers OCT4 and SOX2 (A) in NUMA+ regions and for the sertoli cell marker, GATA4, (B). (A) Cross-section of a human fetal testis (right) with positive immunostaining for OCT4 and SOX2 in a small number of cells. OCT4 and SOX2 staining cross-sections of mouse testes after transplantation of iAZF1, H1 hESC, iAZFΔc, iAZFΔbc and iAZFΔa cell lines respectively. Nuclei are counterstained with DAPI (blue). (B) GATA4 staining in nuclei of putative sertoli cells indicated by asterisks (*). NUMA staining in adjacent sections confirms presence of donor cells. Areas in rectangles were magnified in lower two panels for each line to indicate that GATA4-positive cells are not positive for VASA and NUMA. Nuclei are counterstained with DAPI (blue). Scale bar, 50 μm.

FIG. 24. Morphological patterns of testes xenografted with female H9 (XX karyotype) (A) Testis xenografts were analyzed by Haemotoxylin & Eosin staining for gross histology of interstitial tumors. Arrows indicate embryonal carcinoma-like regions; * indicate yolk sac tumor-like regions. Scale bar, 500 μm. (B) immunohistochemistry was performed on adjacent tissue sections to those used in (A) using NUMA, Sox2 and Oct4 antibodies. Shown are representative images of xenografts from each cell line with NUMA-positive regions (red; top) colocalized with Sox2 (green; middle) and Oct4 (red; bottom) All sections were counterstained with DAPI (blue). Scale bar, 100 μm.

FIG. 25. Transplantation of pre-differentiated iPSC and hESC into mouse spermatogonial tubules. AZF-intact human iPSC cells (iAZF1) and ES cells (H1 hESC) that were transduced with a VASA:GFP reporter were differentiated for 7 days in the presence of BMP4 and BMP8, Retinoic acid and LIF and then FACs-sorted for VASA-GFP+ cells. Subsequently, unsorted (A) and GFP+ sorted (B) cells were injected into seminiferous tubules of busulfan-treated mouse testes (n=8). After 2 months, at least 6 testis xenografts were analyzed by performing whole-mount and cross sectional immunohistochemistry using an anti-human antibody to detect donor cells. (A & B) Representative images and magnified regions in white rectangles are shown for each transplanted population. Arrows indicate surviving donor-derived cells in each image. Nuclei were counterstained with DAPI (blue). (C) A representative cross section from xenografts of differentiated, unsorted iAZF1 cells stained for anti-human NUMA (red), VASA (green) and counterstained with DAPI. Area in white rectangles is magnified in lower panel. Xenografts were also analyzed for 5-methylcytosine (5 MC), 5 hydroxmethylcytosine (5 hMC), Synaptonemal Complex protein-3 (SCP3) and phosphorylated histone H2AX (γH2AX). (D) DNA methylation analysis of testis xenografts after transplantation of pre-differentiated iAZF1 cells with immunostaining for NUMA, VASA, 5 MC, 5 hMC. Areas in white rectangles are shown at higher magnification below each panel. Dotted ovals indicate cells with different 5 MC and 5 hMC signals. (E) Meiosis analysis of mouse testis xenografts after transplantation of pre-differentiated iAZF1 cells with immunostaining for NUMA, VASA and SCP3. Scale bars, 50 μm. Dashed lines always indicate edges of spermatogonial tubules.

### DETAILED DESCRIPTION OF THE INVENTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a peptide” includes a plurality of such polypeptides, and reference to “the primordial germ cell” includes reference to one or more primordial germ cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Methods are provided for the generation of male germ cells from somatic cells. Included are methods of non-integrative reprogramming for germ cell differentiation with a reduced risk of neoplasia during in vivo differentiation. Also included are methods of generating male germ cells from reprogrammed pluripotent cells by direct injection of the undifferentiated reprogrammed cells into the seminiferous tubules. In some embodiments the somatic cells are derived from a male with oligospermia or azoospermia, which may be non-obstructive azoospermia (NOA) of known or unknown origin. In some such embodiments, the azoospermia results from a genetic abnormality in the region at human chromosome Yq11 referred to as the “Azoospermia Factor” (AZF) region.

Non-obstructive azoospermia (NOA) is a heterogeneous disorder that is characterized by various testicular tissue alterations. Such changes result in poor and/or absent spermatogenesis within the testes and the absence of sperm in the ejaculate. NOA accounts for approximately 60% of men with azoospermia and represents the most severe form of male factor infertility. NOA is generally divided into two major categories: pre-testicular and testicular. Genetic testicular causes of NOA include the following: i) chromosomal abnormalities, ii) Y chromosome microdeletions, iii) failure of the primordial germ cells to reach the developing gonads, iv) lack of differentiation of the primordial germ cells to spermatogonia, and v) male germ line mutations that affect spermatogenesis. The lattermost cause is further divided into mutations that control transcription, signal transduction, apoptosis, cell response to stress factors, cytokines (cross-talk), immune sensitization of germ cells, meiotic divisions, and epigenetic factors. Genetic mutations of androgen receptors are also included in this category. For purposes of the present invention, individuals treated for NOA include, without limitation, genetic causes as well as idiopathic cases.

Specific genetic mutations that can give rise to azoospermia include non-mosaic, (47,XXY) and mosaic (47,XXY/46,XY) Klinefelter syndrome; XX males; mutations in X-linked USP26; X-linked SOX3 mutations; Noonan or Noonan-associated syndromes; 45X/46,XY mosaicism (mixed gonadal dysgenesis); Y chromosome microdeletion; deletions in the AZF region, autosome translocations; DAZL mutations; androgen receptor mutations; steroidogenic acute regulatory protein (StAR) mutations; 3BHSD type 2 deficiency; SRD5A2 mutation

Of particular interest for the methods of the invention are genetic mutations in the AZF region, including one or more of AZFa, AZFb and AZFc. AZFa region is the smallest portion of the AZF and spans approximately 400-600 kb of DNA. This subregion contains three genes: USP9Y, DBY (DDX3Y) and UTY. Two protein-coding genes are directly related to male infertility: USP9Y and DBY (recently termed DDX3Y). Complete and partial deletions of AZFa have been described. Complete deletions that remove both genes cause Sertoli cell-only syndrome (SCOS) and bilateral small-sized testes. Partial deletions have also been reported, with particular involvement of USP9Y.

The AZFb subregion spans approximately 6 Mb and is located in the distal portion of interval 5 and the proximal portion of interval 6 (subinterval 5O-6B). The meiotic arrest of spermatogenesis at the primary spermatocyte stage is usually observed when AZFb is deleted. AZFb contains 32 genes and overlaps with AZFc. As such, AZFb deletions often remove certain genes from the AZFc region (e.g., DAZ1 and DAZ2), as well as one copy each of BPY2, CDY1, and PRY. The primary protein-encoding genes in AZFb are RBMY and PRY. Six copies of RBMY are located in the distal portion of AZFb and are only expressed in germ cells. RBMY encodes four types of testis-specific RNA-binding proteins that are involved in mRNA processing, transport, and splicing.

AZFc spans over 3.5 Mb and contains a large number of amplicons that are arranged as direct repeats, inverted repeats, or palindromes. Seven distinct gene families, encompassing 23 genes, are observed in the AZFc region. These families include PRY (two copies), TTY (eight copies), BPY (three copies), DAZ (four copies), GOLGA2LY (two copies), CSPYG4LY (two copies), and CDY (two copies). Deletions in the AZFc region alone or deletions in this region that are combined with deletions in other AZF regions are the most common types and account for as many as 87% of Yq microdeletions. The incidence of these deletions is 1/4,000 males. Although AZFa and AZFb deletions result in azoospermia, deletions in the AZFc region can result in either azoospermia or oligozoospermia.

In some embodiments of the invention, an individual is tested for the presence of a genetic basis for azoospermia prior to reprogramming of somatic cells, where an individual shown to have a mutation, e.g. deletion, in an AZF region is selected for germ cell reconstitution by the methods of the invention. Given that males with deletion of the AZF regions of the long arm of the Y chromosome are infertile, the deletions are generally de novo and are therefore absent from the father of the proband. In azoospermia, genetic testing using karyotyping, Yq chromosome microdeletion analysis and CFTR mutation screening reveals a genetic etiology in approximately 30% of cases.

There are three groups of genetic tests conventionally used to detect genetic diseases in azoospermic men: a) cytogenetic tests that detect chromosomal aneuploidy and structural alterations, such as conventional karyotyping; b) polymerase chain reaction to detect Y chromosome microdeletions; and c) specific gene sequencing for mutational analysis of a specific gene.

Cytokinetic tests are based on analysis of a metaphase spread from a suitable cell population, usually blood cells. The Y chromosome microdeletion (YCMD) assay is a PCR-based blood test that detects the presence or absence of defined sequence-tagged sites (STSs). This technique therefore enables the detection of the presence or absence of any clinically relevant microdeletion. Yq microdeletion analysis is generally performed using multiplex polymerase chain reaction (PCR) to amplify the AZFa, AZFb, and AZFc loci in the long arm of the Y chromosome. Various high throughput methods can be used for specific gene sequencing and mutational analyses. Genetic testing in azoospermic males allows couples to make educated decisions regarding their choice to use a sperm donor or to opt for advanced assisted conception techniques. Such techniques can be coupled with preimplantation genetic diagnosis (PGD) if an abnormal result is obtained.

Germ Cells.

A germ cell is a progenitor cell that will give rise to the gametes of an organism, that is, sperm and egg.

One example of a germ cell is a primordial germ cell (PGC). A PGC is a diploid cell of the germ cell lineage that has the capacity to self-renew as well as differentiate into gametes. PGCs are derived in the yolk sac of the embryo, and migrate through the mesentery of the gut to take up residence in the gonadal ridge, the anlage for the mature gonads. Characteristics of primordial germ cells include their expression of the germ-cell specific genes VASA/DDX4, DAZL, PRDM1/BLIMP1, and DPPA3/STELLA; the hypomethylated state of their genomic DNA both globally and specifically at imprinted loci including H19, PEG1/MEST, SNRPN, and KCNQ; and their ability to give rise to haploid germ cells under certain culture conditions and embryonic germ (EG) cells under others. EG cells are dense, multilayered colonies of cells that express SSEA-1, SSEA-3, SSEA-4, TRA1-60, TRA1-81, and alkaline phosphatase, that are capable of self-renewal, and that can be further cultured to give rise to embryoid bodies comprising derivatives of all three primary germ layers—endoderm, mesoderm and ectoderm (Geijsen, N. et al. (2004) Nature 427: 148-154; West, J. A. et al. (2009) Nature 460: 909-913).

Another example of a germ cell is a “late-stage germ cell”. A late-stage germ cell is any cell of the germ cell lineage that is more differentiated than a primordial germ cell. In other words, a late-stage germ cell is a cell that has already passed through the primordial germ cell stage. An example of a late-stage germ cell would be a cell that has entered meioisis, for example a spermatocyte or oocyte. Late-stage germ cells may express one or more genes associated with meiosis, including γH2AX (an indicator of meiotic recombination) and SCP3 (an indicator of synaptonemal complex formation in meiotic prophase 1). Late-stage germ cells may have a DNA content of 1N, that is, a haploid DNA content. Late-stage germ cells may demonstrate increased methylation of genomic imprinting loci relative to their primordial germ cell ancestor, and may demonstrate differential methylation of these loci relative to their pluripotent, e.g. ES cell or iPS cell, ancestor. Finally, late stage germ cell may express mature gamete markers, for example TEKT1 and ACR, which mark the spermatid to spermatazoan stages of male gamete differentiation; such markers will be known to one of ordinary skill in the art.

In embodiments of the invention, a pluripotent cell is brought into contact with a Sertoli cell environment, in which the pluripotent cell is induced to undergo spermatogenesis, including without limitation one or more, preferably all of the cell types: Type Ad spermatogonium, Type Ap spermatogonium, Type B spermatogonium, Primary spermatocyte and Secondary spermatocyte.

Pluripotent cells. A pluripotent cell is any cell having the ability to differentiate into multiple types of cells in an organism. A reprogrammed, or induced pluripotent cell is derived from a somatic cell, which is reprogrammed to pluripotency by exposure to a cocktail of factors. Examples of methods of generating and characterizing iPS cells may be found in US Application No. 20090047263; US Application No. 20090068742 and U.S. Application No. 61/276,112, which are incorporated herein by reference. iPS cells can be cultured over a long period of time while maintaining the ability to differentiate into ectoderm, mesoderm, or endoderm tissues in a living organism. In some embodiments the methods of the invention include derivation of an induced pluripotent cell from a somatic cell. In other embodiments the methods of the invention utilize an already reprogrammed pluripotent cell.

Reprogramming factors, as used herein, refers to one or a cocktail of biologically active polypeptides or small molecules that act on a cell to alter transcription, and which when expressed reprogram a somatic cell to pluripotency. In some embodiments of the present invention, it is desirable that the reprogramming factors be non-integrating, i.e. provided to the recipient somatic cell in a form that does not result in integration of exogenous DNA into the genome of the recipient cell.

In some embodiments the reprogramming factor is a transcription factor, including without limitation, Oct3/4; Sox2; Klf4; c-Myc; and Nanog. Also of interest as a reprogramming factor is Lin28, which is an mRNA-binding protein thought to influence the translation or stability of specific mRNAs during differentiation.

A Klf4 polypeptide is a polypeptide comprising the amino acid sequence that is at least 70% identical to the amino acid sequence of human Klf4, i.e., Kruppel-Like Factor 4 the sequence of which may be found at GenBank Accession Nos. NP_004226 and NM_004235. Klf4 polypeptides, e.g. those that are at least 70%, 75%, 80%, 85%, 90%, 91%, 92%, 95%, 97%, 99%, or 100% identical to the sequence provided in GenBank Accession No. NM_004235, and the nucleic acids that encode them find use as a reprogramming factor in the present invention.

A c-Myc polypeptide is a polypeptide comprising an amino acid sequence that is at least 70% identical to the amino acid sequence of human c-Myc, i.e., myelocytomatosis viral oncogene homolog, the sequence of which may be found at GenBank Accession Nos. NP_002458 and NM_002467. c-Myc polypeptides, e.g. those that are at least 70%, 75%, 80%, 85%, 90%, 91%, 92%, 95%, 97%, 99%, or 100% identical to the sequence provided in GenBank Accession No. NM_002467, and the nucleic acids that encode them find use as a reprogramming factor in the present invention.

A Nanog polypeptide is a polypeptide comprising an amino acid sequence that is at least 70% identical to the amino acid sequence of human Nanog, i.e., Nanog homeobox, the sequence of which may be found at GenBank Accession Nos. NP_079141 and NM_024865. Nanog polypeptides, e.g. those that are at least 70%, 75%, 80%, 85%, 90%, 91%, 92%, 95%, 97%, 99%, or 100% identical to the sequence provided in GenBank Accession No. NM_024865, and the nucleic acids that encode them find use as a reprogramming factor in the present invention.

A Lin-28 polypeptide is a polypeptide comprising an amino acid sequence that is at least 70% identical to the amino acid sequence of human Lin-28, i.e., Lin-28 homolog of C. elegans, the sequence of which may be found at GenBank Accession Nos. NP_078950 and NM_024674. Lin-28 polypeptides, e.g. those that are at least 70%, 75%, 80%, 85%, 90%, 91%, 92%, 95%, 97%, 99%, or 100% identical to the sequence provided in GenBank Accession No. NM_024674, and the nucleic acids that encode them find use as a reprogramming factor in the present invention.

An Oct3/4 polypeptide is a polypeptide comprising an amino acid sequence that is at least 70% identical to the amino acid sequence of human Oct 3/4, also known as Homo sapiens POU class 5 homeobox 1 (POU5F1) the sequence of which may be found at GenBank Accession Nos. NP_002692 and NM_002701. Oct3/4 polypeptides, e.g. those that are at least 70%, 75%, 80%, 85%, 90%, 91%, 92%, 95%, 97%, 99%, or 100% identical to the sequence provided in GenBank Accession No. NM_002701, and the nucleic acids that encode them find use as a reprogramming factor in the present invention.

A Sox2 polypeptide is a polypeptide comprising the amino acid sequence at least 70% identical to the amino acid sequence of human Sox2, i.e., sex-determining region Y-box 2 protein, the sequence of which may be found at GenBank Accession Nos. NP_003097 and NM_003106. Sox2 polypeptides, e.g. those that are at least 70%, 75%, 80%, 85%, 90%, 91%, 92%, 95%, 97%, 99%, or 100% identical to the sequence provided in GenBank Accession No. NM_003106, and the nucleic acids that encode them find use as a reprogramming factor in the present invention.

VASA.

A VASA polypeptide is a polypeptide comprising the amino acid sequence at least 70% identical to the amino acid sequence of human VASA, also referred to as DEAD/H BOX 4; DDX4, the sequence of which may be found at GenBank Accession Nos. NM_001166534.1; NM_001166533.1; NM_001142549.1 or NM_024415.2. VASA polypeptides, i.e. those that are at least 70%, 75%, 80%, 85%, 90%, 91%, 92%, 95%, 97%, 99%, or 100% identical to the sequence provided in these GenBank Accession Nos., and the nucleic acids that encode them find use as in the generation of male germ cells in the present invention.

VASA encodes a member of the DEAD box family of ATP-dependent RNA helicases.

The deduced 724-amino acid VASA protein contains the 8 conserved domains found in all known DEAD box proteins. VASA has a glycine-rich N terminus with multiple repeats of an RGG motif believed to function in RNA binding. Northern blot analysis of fetal and adult tissues demonstrated that expression of a predominant 2.6-kb VASA transcript is restricted to the ovary and testis. The VASA protein is cytoplasmic and expressed in migratory primordial germ cells in the region of the gonadal ridge in both sexes, consistent with an evolutionarily conserved role in germ cell development.

Non-Integrative Reprogramming.

In some embodiments of the invention, somatic cells are reprogrammed to pluripotency through non-integrative means, i.e. the genome of the cell is not permanently modified. Various methods have been described in the art for this purpose, for example as reviewed by Hayes and Zavazava (2013) Methods Mol Biol. 1029:77-92; and by Gonzalez et al. (2011) Nature Reviews Genetics 12, 231-242, each incorporated by reference. Methods of non-integrative reprogramming include, inter alia, integration followed by excision, typically by employing polycistronic reprogramming cassettes and flanking such polycistronic cassettes with loxP or piggyBac recognition sequences. Thus, these strategies allow for excision of the entire transgene cassette, limiting the potential for the integration of exogenous transgenes to have detrimental effect.

In certain embodiments of the invention, transfection of mRNA or miRNA is used to reprogram cells. As demonstrated herein, multiple rounds of transfection are used, e.g. at least 4, at least 5, at least 6 rounds of transfection with mRNA is used. In some embodiments a modified mRNA is preferred. Key features to modify mRNAs are: i) incorporation of modified ribonucleoside bases for mRNA synthesis, for example substitution of 5-methylcytidine [5mC] for cytidine and pseudouridine [psi] for uridine); ii) treatment with phosphatase of the synthesized RNA molecules after in vitro transcription (ivT). mRNA molecules bearing 5′ triphosphates can be detected by the ssRNA sensor RIG-I and lead to global repression of protein translation, both member of the cell's innate immune response; iii) incorporation of an anti-reverse cap analog (ARCA) during ivT reaction to properly cap mRNA molecules at their 5′ terminus resulting in the initiation of protein translation. Cytoplasmic mRNA contains a cap structure [5₇G(5′)ppp(5′)N, with N=any nucleotide] at its 5′ end that is essential for recognition by the initiation factor eIF4F complex. To further increase cell viability during treatment with exogenous mRNA the culture media may be supplemented with B18R protein, a vaccinia virus decoy receptor for type 1 interferons.

During the process of reprogramming, the somatic cells are transfected repeatedly with the mRNA cocktail. An effective dose of the mRNA cocktail is used, usually at a concentration of at least about 0.1 μg/ml, at least about 1 μg/ml, at least about 10 μg/ml. For example, an effective dose may be at least about 0.01 ng/cell, at least about 0.05 ng/cell, at least about 0.1 ng/cell, at least about 0.5 ng/cell; and may be from about 0.6 to 0.12 ng/cell. Transfection may be performed daily, every two days, etc., for a period of from about 8 days, 10 days, 12 days, 14 days, 16 days, 18 days, 20 days, 22 days etc. until reprogramming is achieved. A pre-defined ratio of factors may be used. The cells can be maintained on feeder layers or in feeder layer-free culture.

In certain specific embodiments the cocktail of reprogramming factors comprises Oct3/4; Sox2; Klf4; and c-Myc (OSKM). The cocktail may further comprise VASA (OSKMV). The ratio of factors may be important to successful reprogramming. Where the cocktail is OSKMV, the ratio may be 3:0.5:1:0.5:1 of the agents. Alternatively, a ratio of OSKM 3:1:1:1; or OSKMV of 3:1:1:1:1 can be used.

Cell penetrating peptide tagged reprogramming factors, which are optionally used in combination with a TLR3 agonist, also find use. Typically, such a polypeptide will comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

Alternatively non-integrating adenoviral vectors, traditional recombinant DNA transfection, transfection of minicircle DNA, or transfection of episomally maintained EBNA1/OriP plasmids can be used to express the cocktail of reprogramming factors. Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line.

The terms “individual,” “subject,” “host,” and “patient,” are used interchangeably herein and refer to any mammalian subject for whom diagnosis, treatment, or therapy is desired, particularly humans.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. In some embodiments the condition being treated is male infertility.

### Reprogramming Somatic Cells to Pluripotency for Generation of Male Germ Cells

In some embodiments of the invention, somatic cells, e.g. adipose tissue cells, bone marrow cells, skin (fibroblast) cells, etc. are obtained from a donor and reprogrammed to pluripotency. The donor may be an individual male mammal, e.g. a human. The donor may be suffering from infertility. Optionally, the donor is screened according to methods known in the art to determine the cause of infertility. In such screening methods, males selected for treatment by the methods of the invention may be determined to have a deletion in the Y chromosome, in particular in one or more of the AZF regions, as described above.

The cells can be reprogrammed by integrative, integrative/excision, or non-integrative methodologies. For therapeutic purposes it is preferable for the resulting pluripotent cells to be free of exogenous DNA.

For reprogramming, a population of somatic cells is contacted with a cocktail of reprogramming factors using any of the above-described technologies for introducing the factors, i.e. modified mRNA, vectors including viral vectors, permeant proteins, etc. Reprogramming factors are described above, and any cocktail that provides for an acceptable conversion to pluripotency may be used. In certain specific embodiments the cocktail of reprogramming factors comprises Oct3/4; Sox2; Klf4; and c-Myc (OSKM). The cocktail may further comprise VASA (OSKMV). As shown herein, the addition of VASA does not affect reprogramming efficiencies or kinetics. However, the addition of VASA to the reprogramming mix significantly reduces the tumorigenic potential of the pluripotent cells in vivo. Reprogrammed cells transiently exposed to VASA are shown to produce clusters of human germ cells in vivo, in the absence of undesirable fibrotic tissue.

The pluripotent cells may be isolated for use in ex vivo or in vitro fertilization, or for transplantation into a recipient, particularly for transplantation back into the somatic cell donor. In such cases, genes are optionally introduced into the pluripotent cells prior to performing the method or into the differentiated germ cells that are produced after performing the method, for example, to replace genes having a loss of function mutation, provide marker genes, etc. Alternatively, vectors may be introduced that express antisense mRNA or ribozymes so as to block expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to introduce nucleic acids into the germ cells, e.g. electroporation, calcium precipitated DNA, fusion, transfection, lipofection, infection and the like, as discussed above. The particular manner in which the DNA is introduced is not critical to the practice of the invention.

Kits may be provided, where the kit will comprise reagents that are sufficient to reprogram somatic cells into pluripotent cells suitable for in vivo differentiation into male germ cells, as described herein. A combination of interest may include one or more agents of the present invention, i.e. a set of OSKMV vectors or modified mRNAs. Kits may also include tubes, buffers, etc., and instructions for use.

Compositions of reprogrammed pluripotent cells, including without limitation pluripotent cells derived from an azoospermic or oligospermic donor, and in particular those donors in which infertility is associated with a deletion in the Y chromosome in the AZF region.

The population of pluripotent cells can be combined with a pharmaceutically acceptable excipient that maintains the viability of the cells. Various media are commercially available and may be used according to the nature of the cells, including dMEM, HBSS, dPBS, RPMI, Iscove's medium, etc., which may be supplemented with HAS, etc. The cell population may be substantially all pluripotent cells, e.g. 80% or more of the cell composition, about 90% or more of the cell composition, about 95% or more of the cell composition, and will preferably be about 95% or more of the cell composition.

The cell population may be used immediately. Alternatively, the cell population may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

For the treatment of infertility, the suspension of pluripotent cells can be injected into the seminiferous tubules of a male being treated for infertility. The dose of cells will be sufficient to provide for a detectable sperm count after a period of time sufficient to allow differentiation of the pluripotent cells, e.g. a total per male of at least 10³, 10⁴, 10⁵, 10⁶, 10⁷, 10⁸, 10⁹ pluripotent cells. The cells may be fractionated, i.e. injected into multiple sites, i.e. 1, 2, 3, 4, 5, 10, 15, 20, 30 or more sites. Any suitable method for injection can be used, e.g. cannulation of efferent ducts, etc. Transplantation may be performed at a single time point, or in a plurality of time points, e.g. 1, 2, 3, 4, 5, 6, 7, 8, 9, 10, 15, 20 etc. time points, over days, weeks, months, etc. as required for the desired treatment of infertility.

For research purposes the suspension of pluripotent cells can be injected into a test animal, e.g. into a busulfan treated mouse or rat, as described in the Examples herein. The cells that differentiate in vivo may be of the same species, e.g. mouse to mouse, etc., and will be expected to differentiate into viable sperm. Where the cells that differentiate are across species, e.g. human to mouse, etc., differentiation will be initiated but will typically not result in sperm production.

Alternatively for research purposes the pluripotent cells may be brought into contact with a Sertoli cell environment in vitro, for example using a culture system known in the art, for example as described by any one of Yokonishi et al. Biol Reprod. 2013; Pan et al. PLoS One. 2013; 8(3):e60054; Aponte et al. Clinics (Sao Paulo). 2013; 68 Suppl 1:157-67; Minaee et al. Acta Med Iran. 2013; 51(1):1-11; Li et al. PLoS One. 2013; 8(2):e56696; etc.

In addition to the treatment of infertility, the male germ cells have many applications in modern biotechnology and molecular biology. They are useful in the production of animals; in the study of gametogenesis and infertility and the provision of pluripotent stem cells for tissue regeneration in the therapy of degenerative diseases and repopulation of damaged tissue following trauma; they are also useful for pharmacological studies and environmental studies of chemical impacts on male germ cells. The male germ cells themselves may be analyzed, for example for the expression of genes, for example to better characterize the cells. The pluripotent cells and male germ cells derived therefrom may be used as a tool for screening agents for activity in modulating the differentiation of these other cell types.

### Screening Assays

Also provided are methods for screening one or more agents for activity in modulating germ cell differentiation. The culture system and compositions described herein provide a useful system to screen candidate agents for activity in modulating germ cell differentiation e.g. by adding a candidate agent to the culture system and assaying for changes in the quantity and/or rate of germ cell differentiation. In screening assays for biologically active agents, a first population of pluripotent cells is contacted with a candidate agent. The characteristics of the contacted cells are then compared with the characteristics of a second population of pluripotent cells that have not been contacted with the agent, wherein differences in the characteristics between the first population and the second population indicate that the candidate agent modulates germ cell differentiation. Prior to contacting the first population of pluripotent cells with the candidate agent, the first population and second population of pluripotent cells are substantially the same. Typically, the first population of pluripotent cells, i.e. the population that was contacted with agent, will be cultured either concurrently with being contacted or subsequent to being contacted under conditions that are permissive for or promote/induce germ cell differentiation. In such cases, the second population of pluripotent cells, i.e. the population that was not contacted with agent, will likewise be cultured under conditions that are substantially the same.

An agent that modulates germ cell differentiation is an agent that modulates, or alters, or affects, the number of germs cells that differentiate in culture or the rate at which the germ cells differentiate in culture relative to the number or rate of germ cells that differentiate in the absence of the agent. Agents that modulate germ cell differentiation may promote, i.e. induce, enhance, or increase, differentiation, or they may inhibit, i.e. prevent, attenuate, or decrease, differentiation. For example, as discussed above, an agent that promotes germ cell differentiation is an agent that induces, enhances, or otherwise increases the quantity/number of germ cells that differentiate in culture and/or the rate at which the germ cells differentiate in culture relative to germ cell differentiation under the same culture conditions but in the absence of the agent. Reciprocally, an agent that inhibits germ cell differentiation prevents, attenuates or decreases the quantity/number of germ cells that differentiate in culture and/or the rate at which the germ cells differentiate in culture relative to germ cell differentiation under the same culture conditions but in the absence of the agent. An agent that modulates germ cell differentiation is one that modulates the quantity/rate of germ cells differentiation by about 1.5-fold, by about 2-fold, by about 2.5-fold, by about 3-fold, by 4-fold, by about 10-fold, by about 20-fold, by about 50-fold, by about 100-fold. As discussed above, agents that may modulate germ cell differentiation include small molecule compounds, nucleic acids, and polypeptides.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype. Positive controls of interest include those genes and polypeptides identified herein as affecting germ cell differentiation.

Characteristics, or parameters, that might be assessed as being reflective of an agent's activity are typically quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Some examples of characteristics, or parameters, that might be assessed include the number of cells that differentiated into germ cells in each population, the genes that are expressed by the cells of the populations, the methylation state of the genomic DNA of the cells of the populations, and the ability of the cells of the populations to give rise to embryonic germ cell populations.

Various methods can be utilized for assessing the characteristics of the cell populations after contact with the candidate agent(s). For measuring the amount of a molecule, e.g. a protein, that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). In some embodiments of the invention, the pluripotent cells are contacted with a polynucleotide comprising a detectable marker under control of a promoter that is selectively active in primordial germ cells prior to culturing in the presence of the candidate agent, which may be used to quantify the number of cells in the population that differentiation into primordial germ cells or isolate the primordial germ cells for better assessing their characteristics.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications that might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Differentiation of Human Germ Cells from Induced Pluripotent Stem Cells In Vitro and In Vivo

Applications in human reproductive biology require reliable methods to differentiate bona fide human germ cells. For this purpose, we derived induced pluripotent stem cells (iPSCs) under xeno-free conditions via use of modified mRNAs that encode the transcription factors (OCT4, SOX2, KLF4 and cMYC (OSKM)) alone or in combination with the germ cell specific mRNA that encodes VASA, an RNA-binding protein (OSKMV). We observed only subtle differences in expression of individual germ cell specific genes, epigenetic profiles and differentiation in vitro with OSKMV relative to OSKM cells. Moreover, when transplanted directly into mouse seminiferous tubules, we observed that both undifferentiated OSKM and OSKMV cells differentiated to human germ cells, with few if any somatic cells formed, within the spermatogenic tubules as indicated by morphology, localization of cells and expression of key germ cell proteins. However, undifferentiated OSKM and OSKMV cells differed in regards to number of germ cells formed and development of masses of undifferentiated/tumorigenic cells. Results indicate that mRNA reprogramming in combination with transplantation directly into seminiferous tubules promotes human germ cell development. Most importantly, the balance between differentiation and proliferation in vivo can be skewed in favor of germ cell differentiation by inclusion of VASA mRNA during reprogramming.

Induction of pluripotency was originally achieved with mouse embryonic fibroblasts with four transcription factors, OCT3/4 (also known as POU5F1), SOX2, KLF4 and cMYC using retroviral vectors for ectopic gene expression; however, other factors are also known to modulate iPSC derivation including the addition of NANOG and LIN28. With the accumulation of additional reprogramming factors (RPFs) and chemical compounds, the identity of derived iPSC lines can be inherently altered. Indeed, we are just beginning to understand how iPSC lines differ from each other and from human embryonic stem cells (hESCs). The quality and phenotype of a derived iPSC line is primarily determined by: i) Identity and number of RPFs, ii) ratio of RPFs to each other, iii) reprogramming strategy and iv) duration of ectopic expression during reprogramming. In fact Carey et al. have shown that even relatively minor differences in expression ratios of reprogramming factors lead to different iPSC line qualities and altered epigenetic states.

It is of particular importance to reproductive biology and medicine to develop reliable methods to differentiate germ cells from iPSCs for basic and potential clinical applications. In this study, we examined the use of mRNA-based reprogramming to produce xeno-free and integration-free iPSCs; we used repeated delivery of modified mRNAs encoding the transcription factors OCT3/4 [O], SOX2 [S], KLF4 [K], and cMYC [M] into human neonatal foreskin and adult dermal fibroblasts over an extended period results in the efficient derivation of mRNA-based integration-free human induced pluripotent stem cells (RiPSCs). During the course of the study, we observed that the number of consecutive transfections can be as few as six, thus limiting manipulation of the cells. Moreover, based on previous studies that documented the ability of germ cell specific proteins to promote germ cell development, we hypothesized that addition of the mRNA encoding for VASA [V] protein, to the Yamanaka mRNA cocktail might impact the reprogramming process and/or the resulting RiPSC lines relative to reprogramming with Yamanaka factors alone. The VASA gene encodes a highly conserved and germ cell specific RNA-binding protein whose role in germ line development remains unclear but has been suggested to act as a chaperone enabling correct folding of different target RNAs in germ cells. We demonstrate that transient, ectopic expression of OSKMV reprogrammed human fibroblasts into a stable pluripotent state that is very similar to RiPSCs reprogrammed without VASA, but distinct in several critical functional assays in vitro and especially in vivo.

### Results

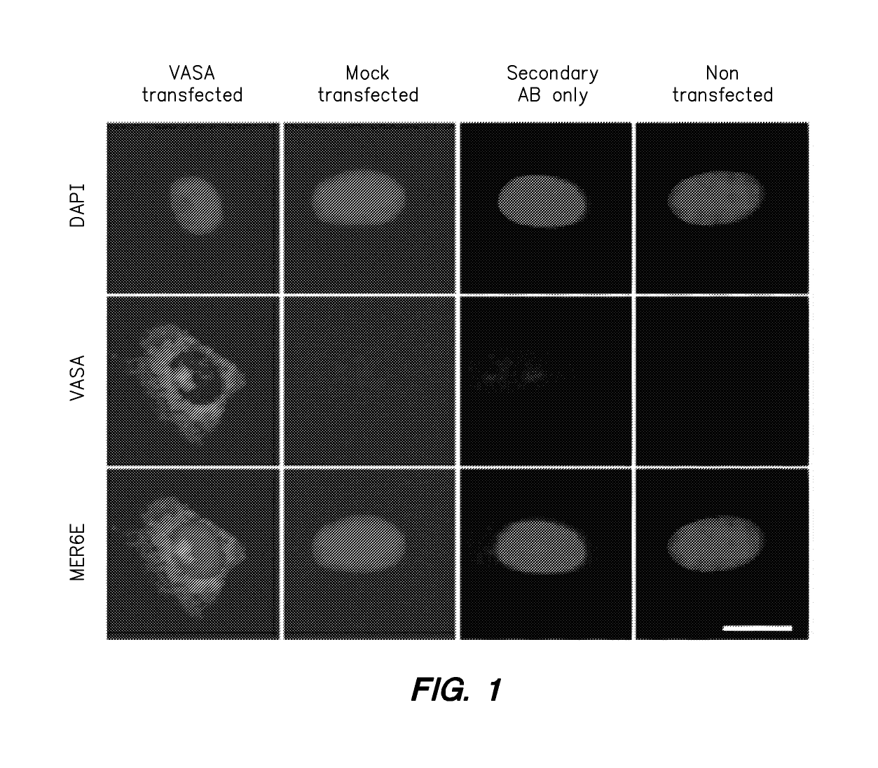

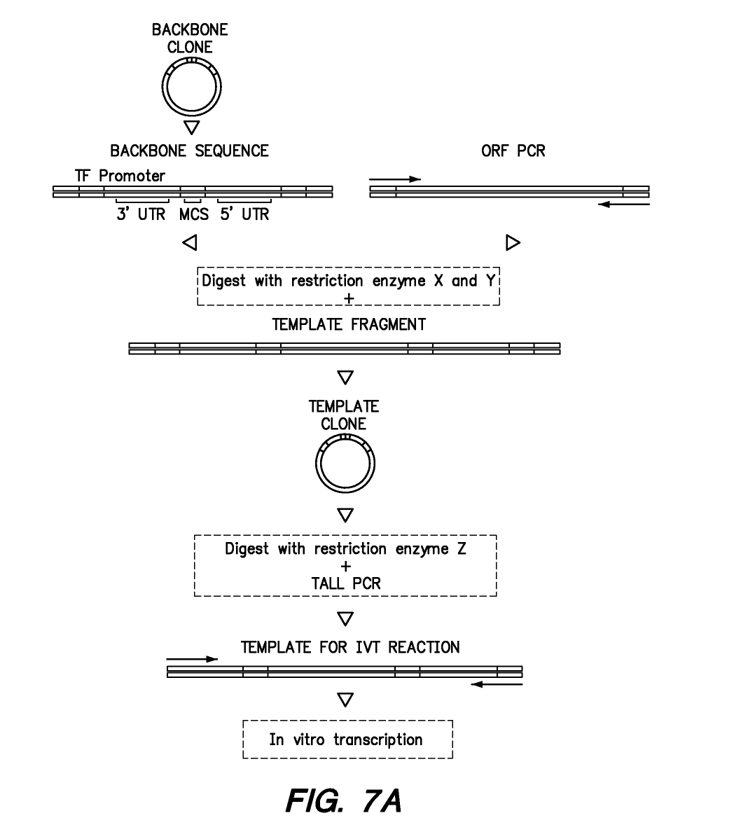

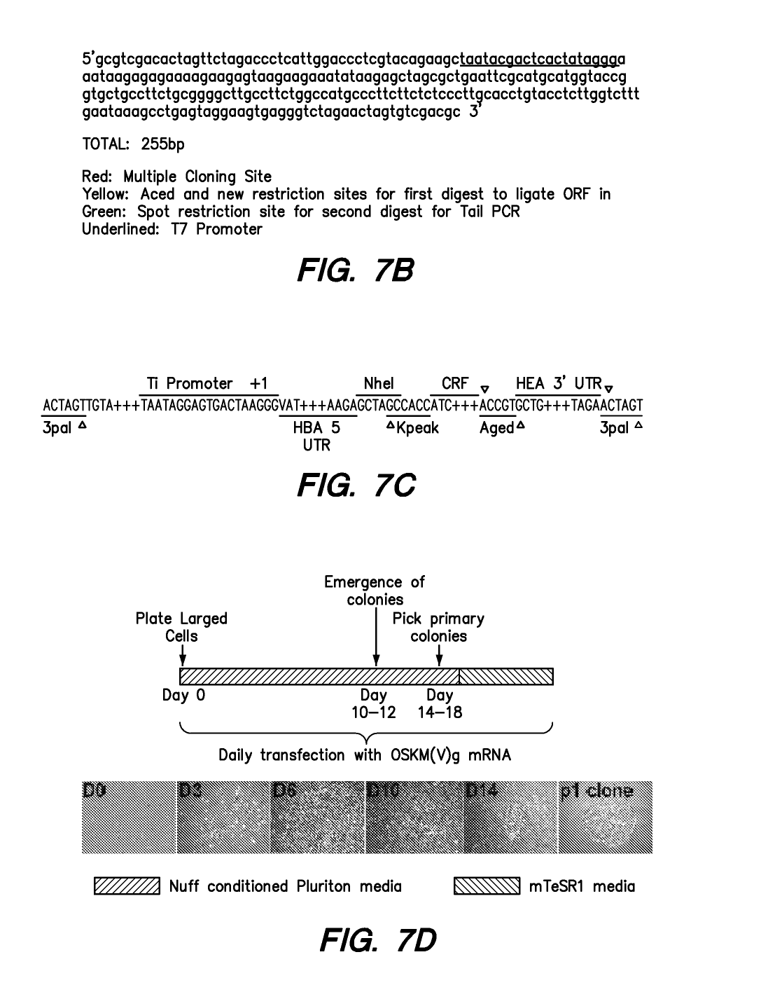

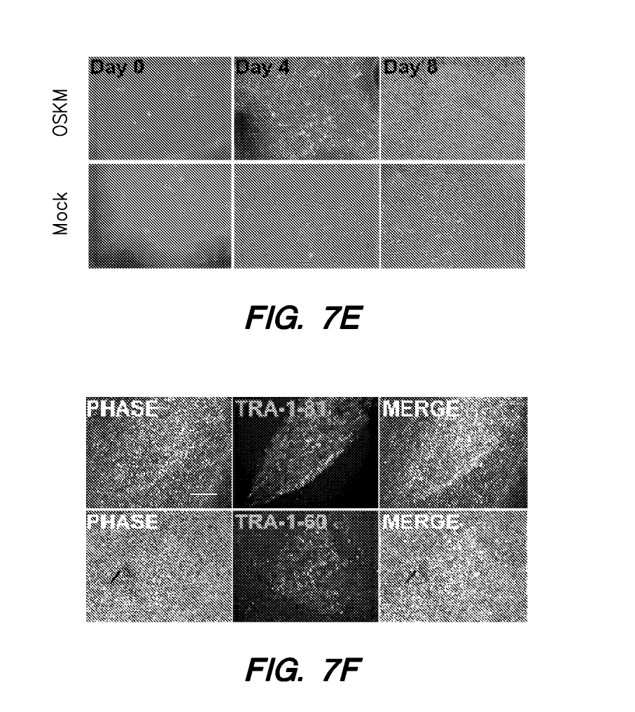

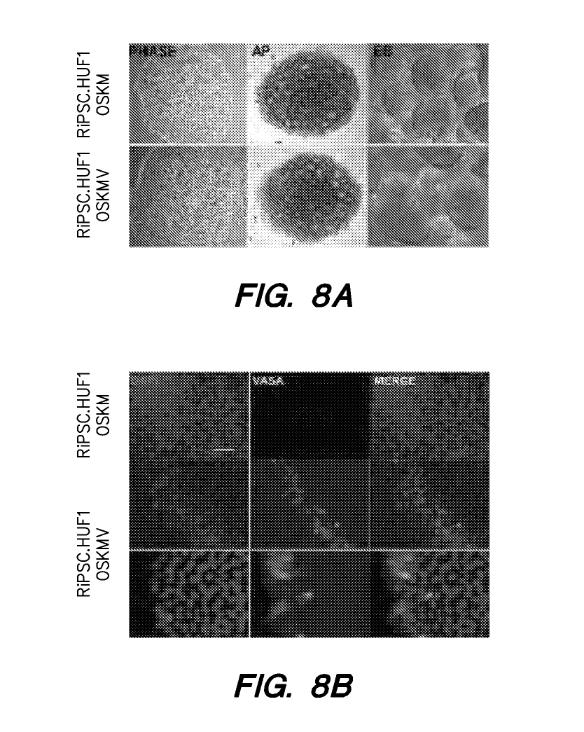

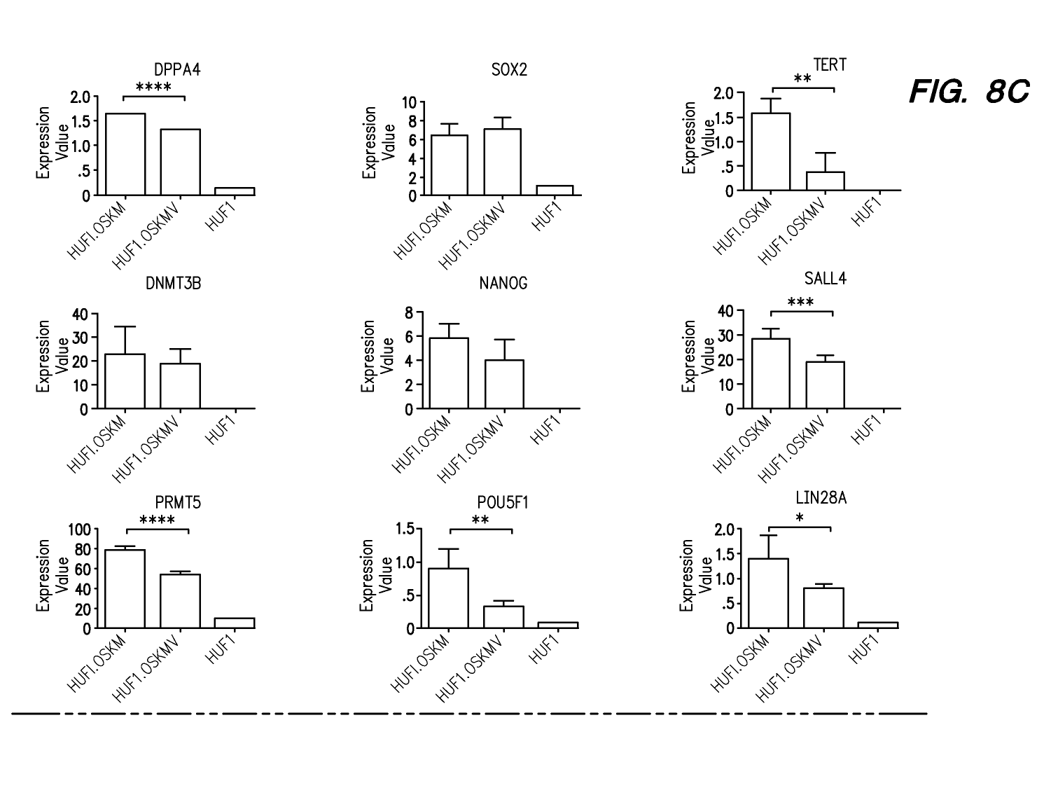

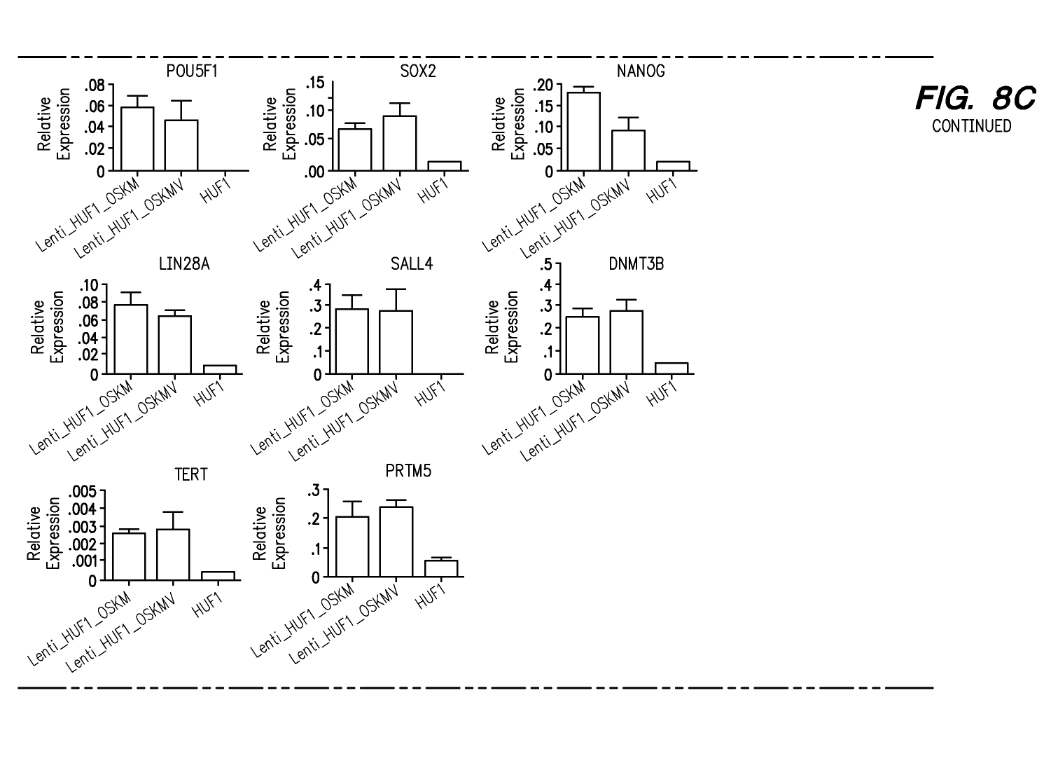

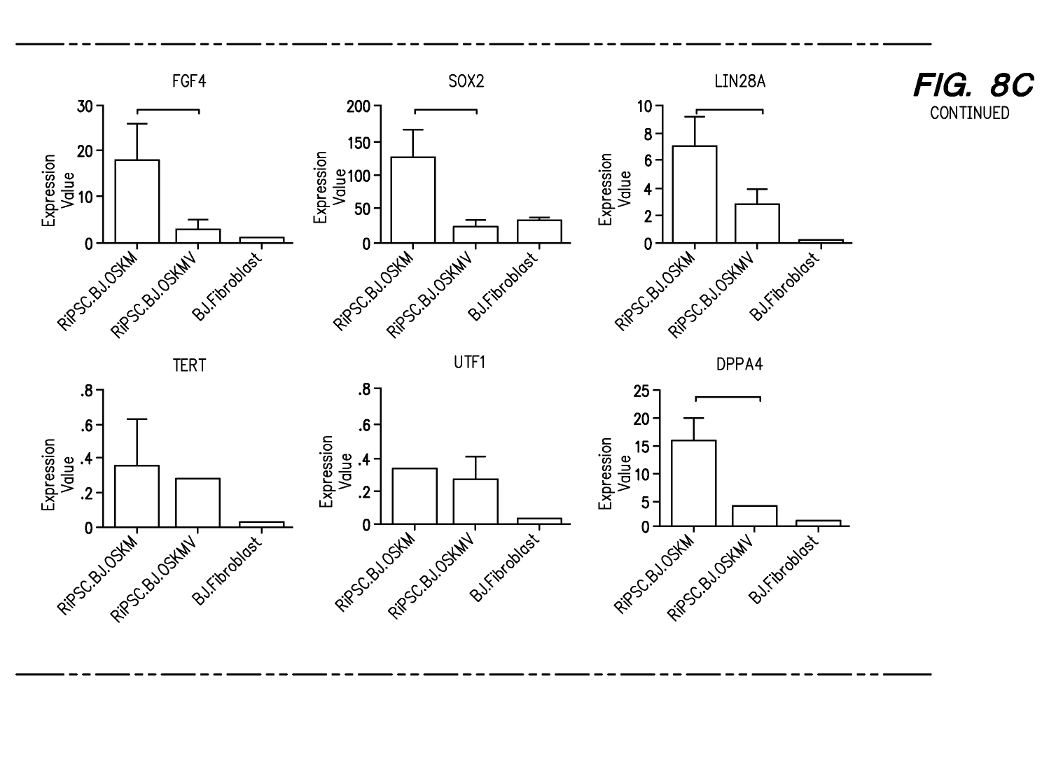

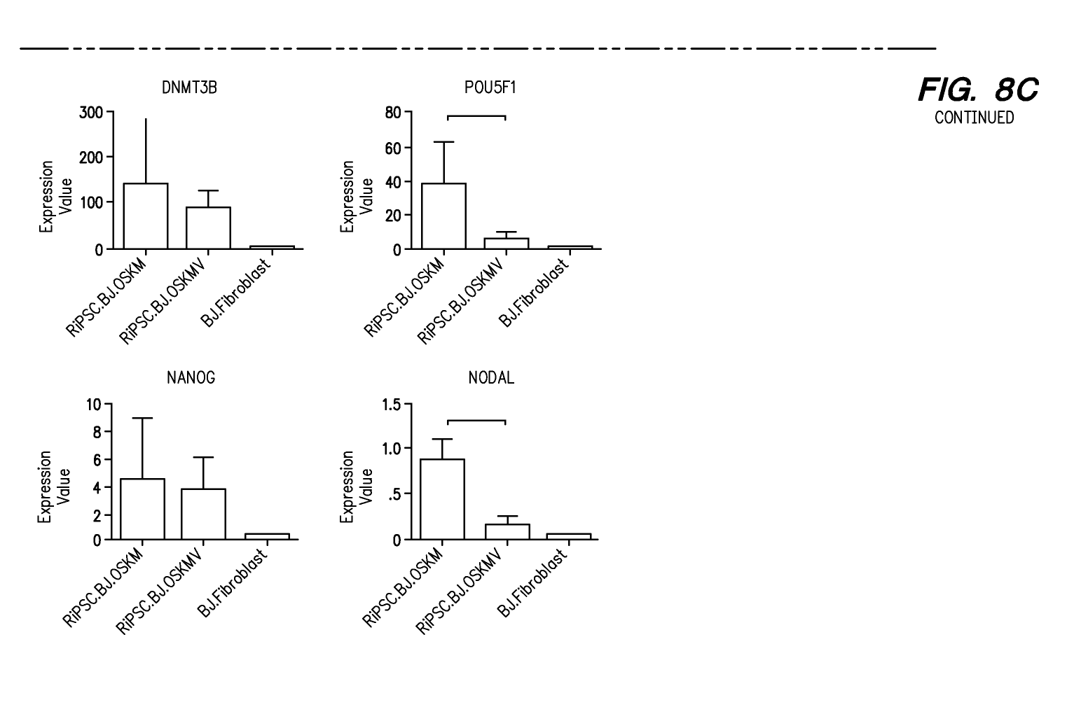

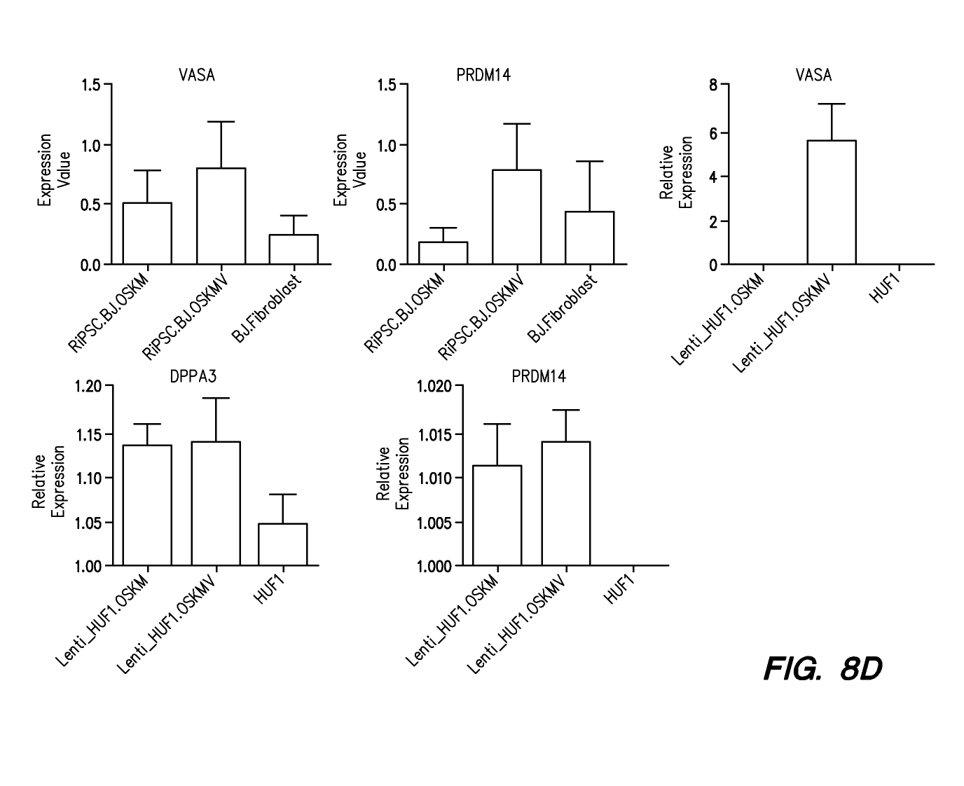

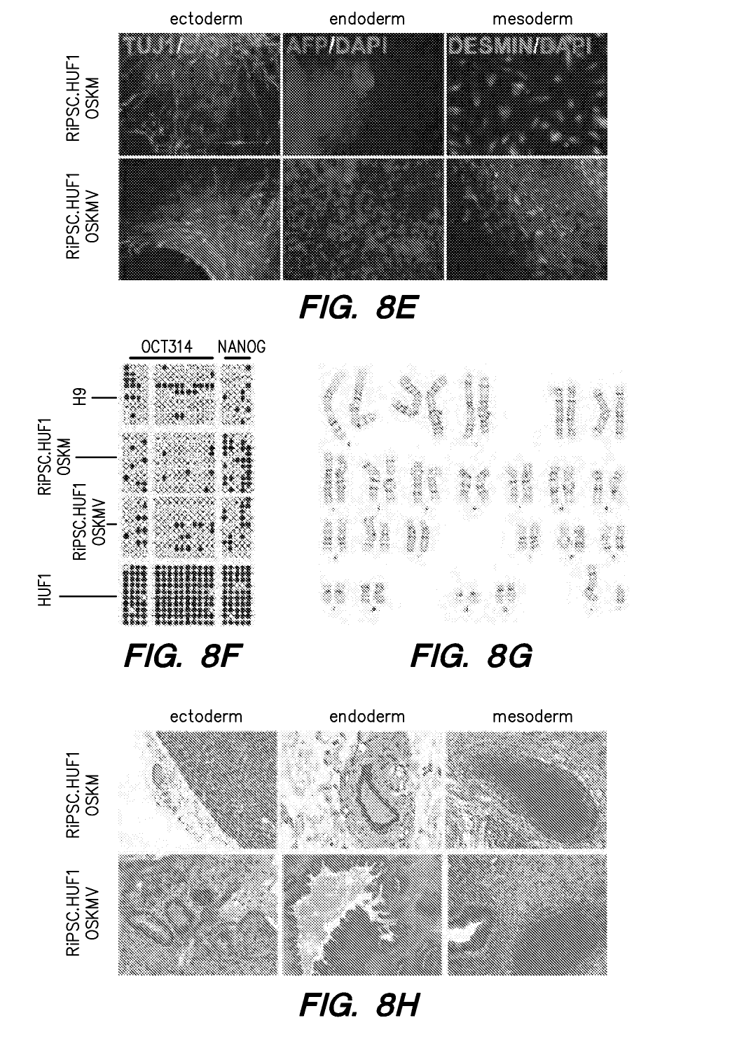

Addition of VASA to OSKM reprogramming mix does not affect reprogramming efficiencies or kinetics. Synthesized modified mRNAs, including VASA mRNA, were produced and validated prior to use as shown (FIG. 6a,b); protein expression and localization was confirmed via immunocytochemistry (FIG. 1). We added synthesized VASA mRNA to OSKM factors in molar ratios of 3:0.5:1:0.5:1 (OSKMV) and examined reprogramming efficiency and kinetics of different somatic lines [BJ (XY), HUF1 (XY), HUF3 (XX), and HUF9 (XX)] with either the OSKM or OSKMV cocktail in parallel. We adapted a previously reported protocol in which feeder-free RiPSCs derivation was accomplished with 6 factors (OSKM+NANOG+LIN28A) and modified OCT3/4. We successfully derived feeder- and xeno-free RiPSCs with OSKM alone and with OSKMV (see FIG. 7a-f). We observed colony formation with both OSKM and OSKMV reprogramming with no significant differences detected in colony number (˜0.5% efficiency) or timing of colony appearance (6-12 days post first-transfection). We then focused our analysis on the XY lines, HUF1 and BJ, and compared OSKM- and OSKMV-derived lines across a series of functional and molecular assays beginning with a comparison of morphology and alkaline phosphatase (AP) staining. We did not observe notable differences, regardless of reprogramming cocktail used (FIG. 8a). Colonies reprogrammed with OSKMV were characterized by a high nucleus/cytoplasm ratio, prominent nucleoli, well defined borders and a distinguishing chromatin structure and nuclear architecture (speckles and heterochromatin domains), all features that are very similar to OSKM reprogrammed colonies and human embryonic stem cells.

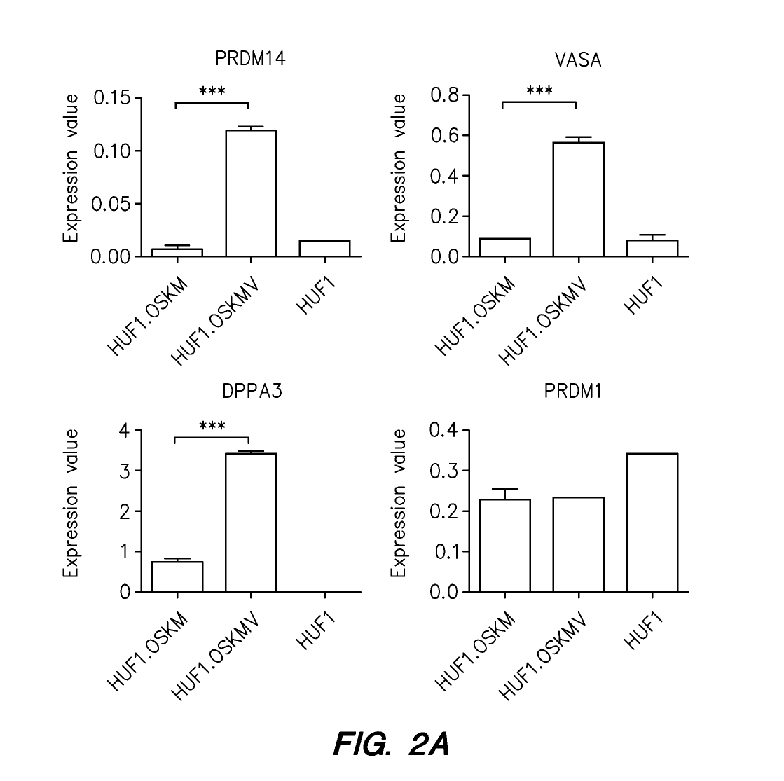

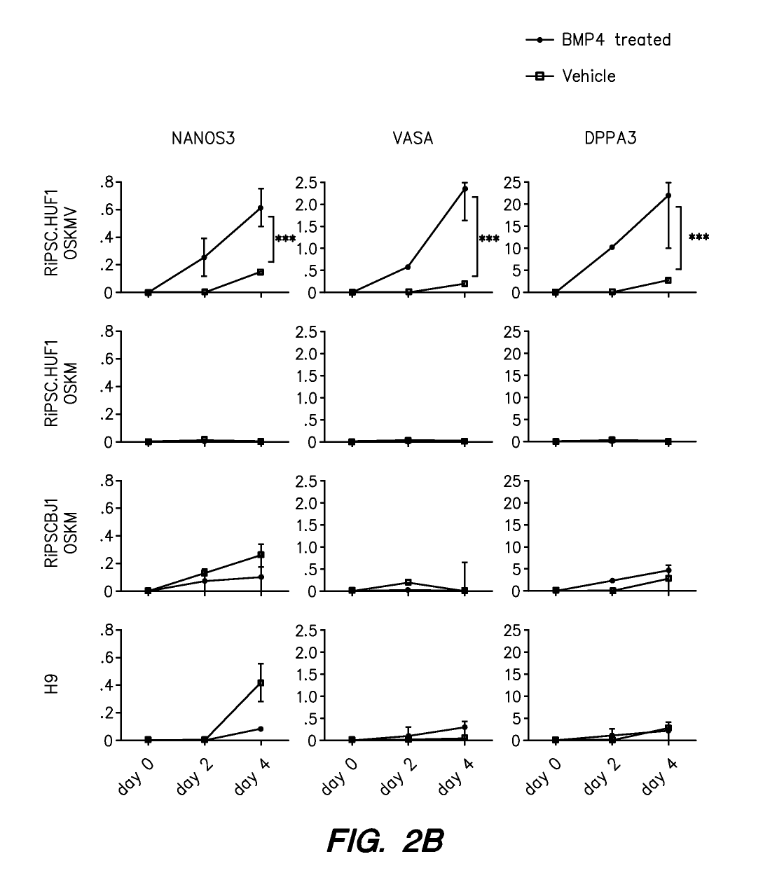

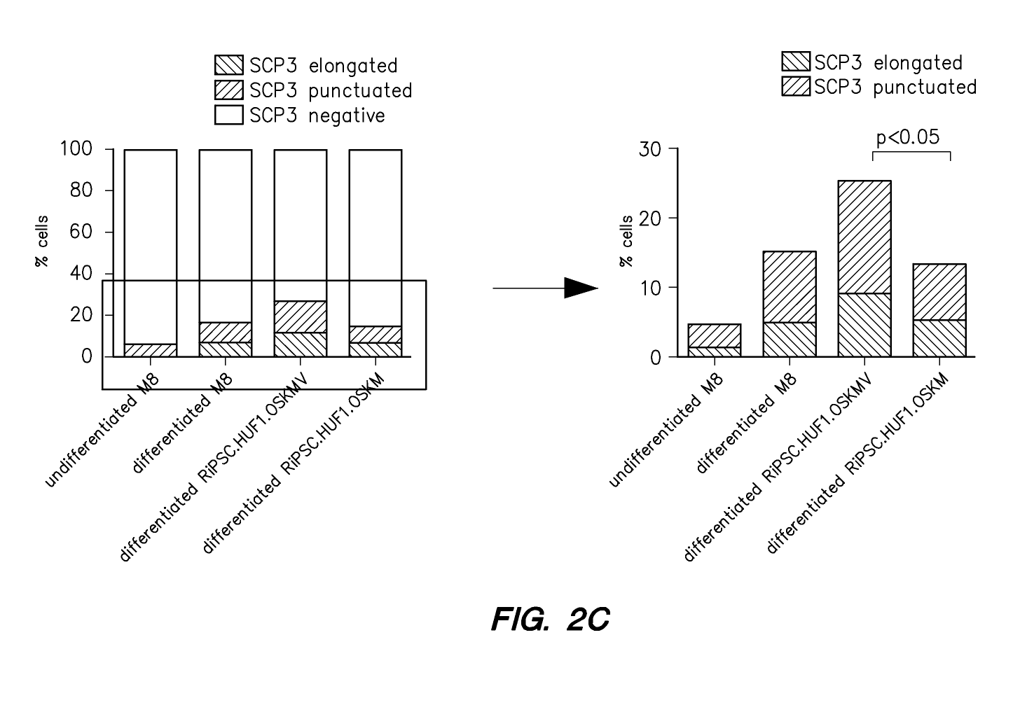

Transient ectopic VASA expression alters gene expression signatures of derived RiPSC lines As part of the assessment of pluripotency, we examined endogenous gene and protein expression of various markers associated with pluripotency including POU5F1, NANOG, SALL4, and DNMT3B in both colony types (FIG. 8c). Notably, expression of the majority of markers associated with pluripotency was significantly lower (p<0.05) in lines reprogrammed with OSKMV relative to their OSKM counterpart, with PRMT5, SALL4, and DPPA4 being the most significantly different (p<0.001). We also confirmed a similar reduction of a subset of markers in lines that were derived with OSKM or OSKMV via a lentiviral reprogramming strategy to exclude reprogramming strategy related events (FIG. 8c). We then examined effects of transient ectopic expression of VASA during reprogramming on expression of genes associated with early germ cell development. We observed that the majority of markers showed gene expression levels similar to the lines reprogrammed with OSKM alone and/or the parental fibroblast line, indicating no gene activation (exemplified by PRDM1). However, a subset (PRDM14, DPPA3 [STELLA], and VASA) was expressed at significantly higher levels (p<0.001) in RiPSC lines reprogrammed with OSKMV relative to OSKM derived colonies (FIG. 2a and FIG. 8d). Again, results were mirrored in the lentiviral-derived HUF1 iPSC line (FIG. 8d). We note that gene expression was measured at two different passages (passage 4 and 14) to eliminate the possibility of expression from exogenous mRNA and to demonstrate stability of the distinct endogenous gene expression profile; we further confirmed endogenous VASA gene expression in OSKMV cells with immunocytochemistry (FIG. 8b).

OSKMV derived lines, similar to OSKM derived lines, are fully pluripotent. To further assess whether the mRNA-reprogrammed lines are fully pluripotent, we performed a variety of molecular and functional assays. Spontaneous differentiation revealed that all OSKMV and OSKM derived RiPSCs can form all three germ layers in vitro (FIG. 8e). Epigenetic analysis revealed similar methylation status of OCT3/4 and NANOG promoters of both OSKM and OKSMV derived RiPSC.HUF1 lines comparable to hESCs, which were substantially different relative to the parental fibroblast line (FIG. 8f). Moreover, we observed that all clones were karyotypically normal as exemplified by RiPSC.HUF1.OSKM (FIG. 8g) and differentiated to all three germ layers in vivo in teratoma assays, following kidney capsule injection into SCID mice (FIG. 8h). Taken together, these results indicate that mRNA derived iPSC clones from multiple independent fibroblast lines are fully reprogrammed, pluripotent and display a molecular and functional phenotype that closely resembles hESCs. Moreover, we show that RiPSC derivation with addition of VASA to OSKM factors leads to a pluripotent state by the most stringent pluripotency assays available to date for human pluripotent cells.

OSKMV and OSKM derived clones respond differently to BMP4 treatment. VASA overexpression from lentiviral vectors has been reported to enhance differentiation into primordial germ cells (PGCs) when overexpressed in both human ESCs and iPSCs. In mice primordial germ cell specification is characterized by BLIMP1 (PRDM1) expression in a small number of cells in the posterior, proximal mouse epiblast at E6.25 followed by upregulation of Stella around E7.25. Several groups demonstrated in vitro differentiation of PGCs from both mouse ESCs and hESCs via media supplementation with bone morphogenic proteins (BMPs). Thus, we hypothesized that lines derived with OSKMV might reveal a different potential in their ability to respond to BMP4 induced differentiation into early germ cells in vitro relative to use of OSKM alone. We supplemented culture media of both OSKM and OSKMV derived lines with BMP4 over the course of four days and assessed activation of gene expression of early germ line markers. We included two controls—an mRNA derived (OSKM) BJ fibroblast line and a hESC line (H9). Although a subset of examined genes (NANOS2, RET, Y chromosome DAZ2 and others) did not show significant changes in gene expression in either the OSKM or OSKMV derived lines, expression of three markers—NANOS3, VASA, and DPPA3—was significantly upregulated (p<0.01) post-BMP4 treatment in the line derived with OSKMV after four days of BMP4 treatment (FIG. 2b). In contrast, lines derived with OSKM as well as control lines did not demonstrate differential expression of these germ cell markers in response to BMP4 treatment, indicating that observed differences are confined to different reprogramming factor cocktails used during RiPSC derivation.

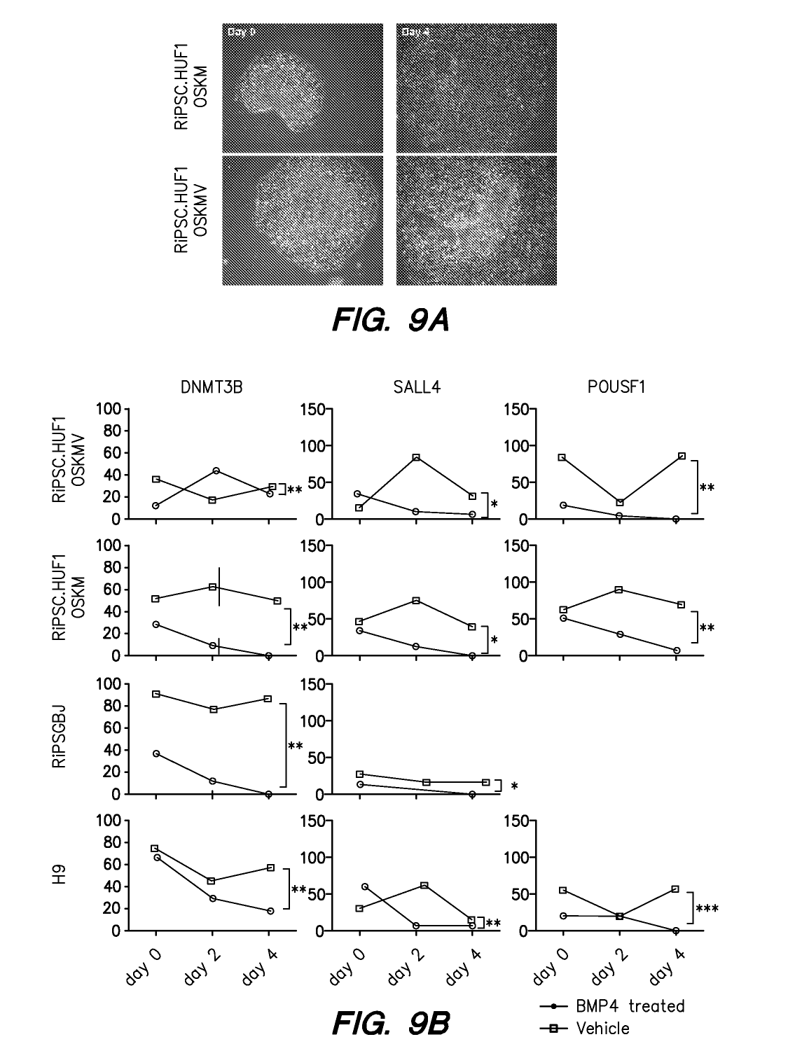

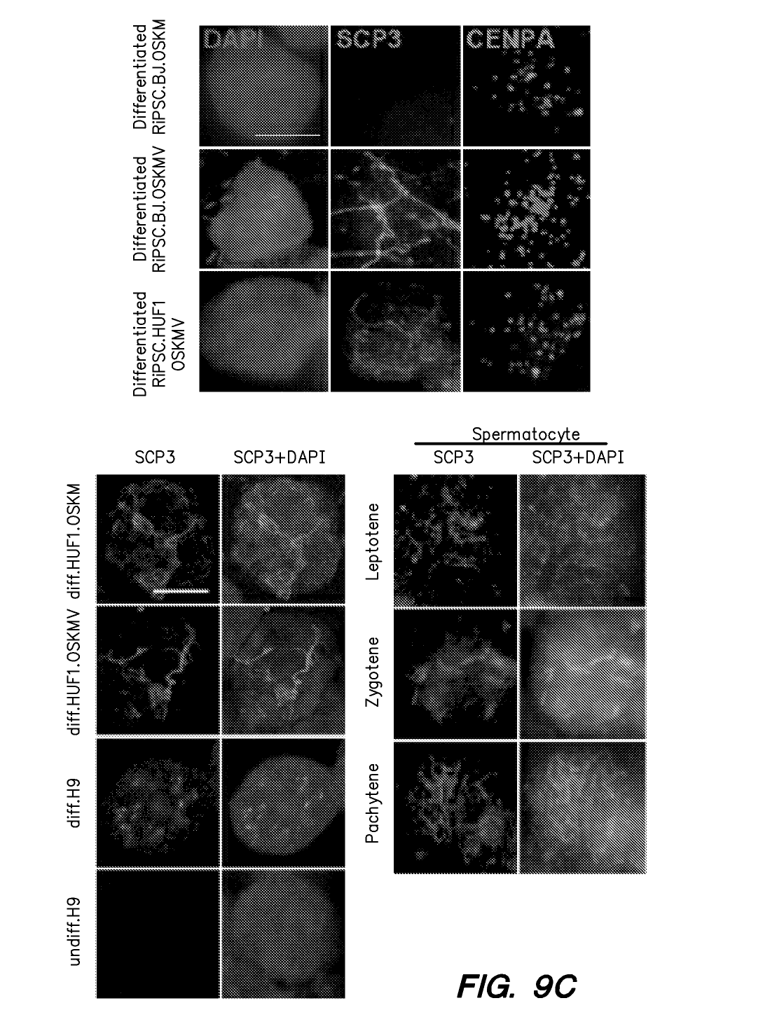

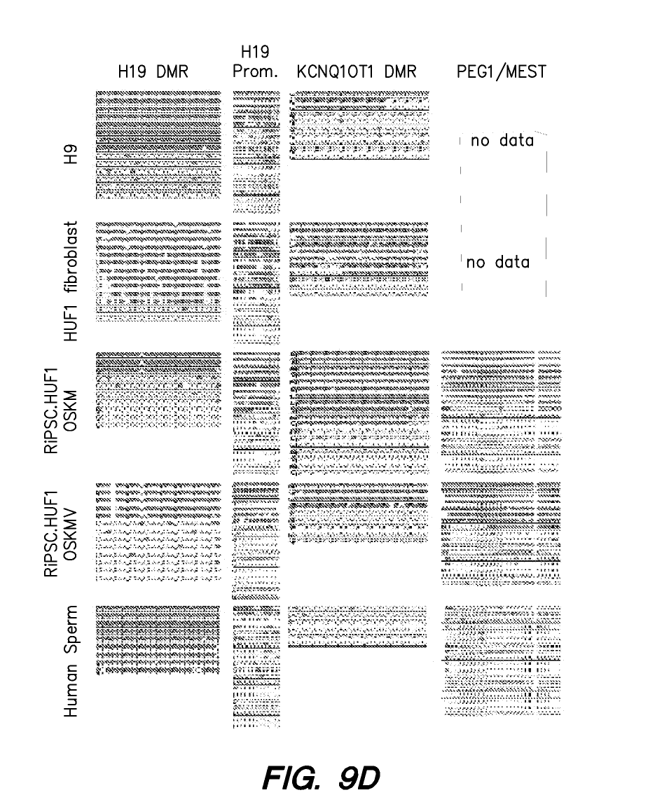

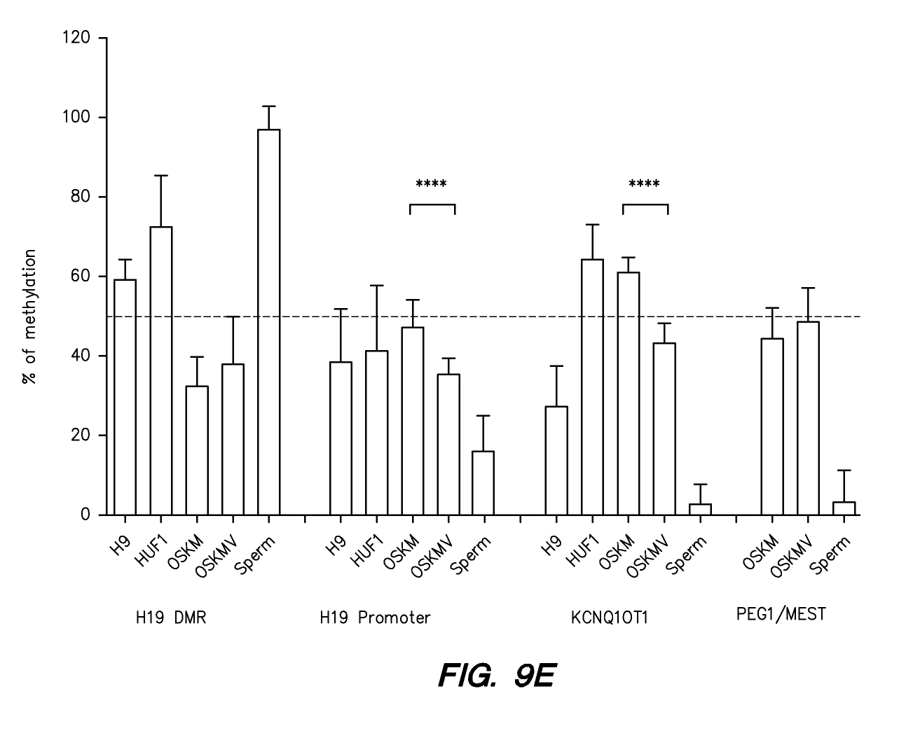

We also examined gene expression changes of pluripotency-associated markers during BMP4 supplementation and expected downregulation based on observed morphology changes during differentiation (FIG. 9a). DNMT3B, POUF51, and SALL4, all genes that are strongly expressed in hESCs and RiPSCs, showed a rapid decrease in gene expression after four days of BMP4 treatment in both OSKM and OSKMV derived lines as well as both control lines (FIG. 9b).

OSKMV and OSKM derived lines display distinct SCP3 staining patterns. Synaptonemal Complex Protein 3 (SCP3) encodes a meiosis-specific protein that is essential for formation of meiotic synaptonemal complexes of the maternal and paternal homologous chromosomes. Its localization and distribution along the chromosomes is a significant indicator of meiotic progression in pluripotent stem cell differentiation to germ cells. Ectopic overexpression of VASA in pluripotent human iPSCs has recently been shown to promote meiotic progression as judged by positive SCP3 staining patterns. Given the results in this study, we reasoned that perhaps OSKMV-derived RiPSCs might be predisposed relative to OSKM-derived RiPSCs to differentiate efficiently to meiotic intermediates with culture in BMP4. Thus, we assessed co-expression of SCP3 and CENPA, a centromeric protein. Our analysis revealed that the majority of cells regardless of VASA mRNA addition during reprogramming stained negative for SCP3 protein indicating no meiotic activity (FIG. 2c and FIG. 9c). Nonetheless, clusters of differentiated cells showed a punctuated staining pattern, indicative of early meiotic events (leptotene stage); moreover, cells reprogrammed with OSKMV displayed slightly higher percentages (p<0.05) of cells positive for SCP3 staining. In addition, we noticed a distinct staining pattern that was most prominent in RiPSCs reprogrammed with OSKMV with elevated SCP3 clusters with elongated structures indicative of late, albeit somewhat disorganized, assembly of meiotic chromosomes in zygotene, pachytene or diplotene meiotic prophase I stages. This staining pattern was observed in approximately 16% of cells, a percentage that is significantly higher (p<0.05) than in the OSKM reprogrammed line or hESC control line (8% and 10%, respectively) (FIG. 2c). Few cells in the undifferentiated negative control (H9) displayed SCP3 staining patterns as expected.

Analysis of epigenetic status and global transcription profiles. We next examined the epigenetic status and global transcriptional profiles of derived lines. As shown (FIG. 9d,e), we analyzed four key imprinted genes via bisulfite sequencing: KCNQ1OT1- and PEG1/MEST-linked differentially methylated regions (DMRs), both maternally imprinted as well as the H19 DMR and H19 promoter loci, both paternally imprinted. Our analysis revealed slight but significant differences (p<0.0001) in methylation status of both maternally- and paternally-imprinted loci (FIG. 9e).

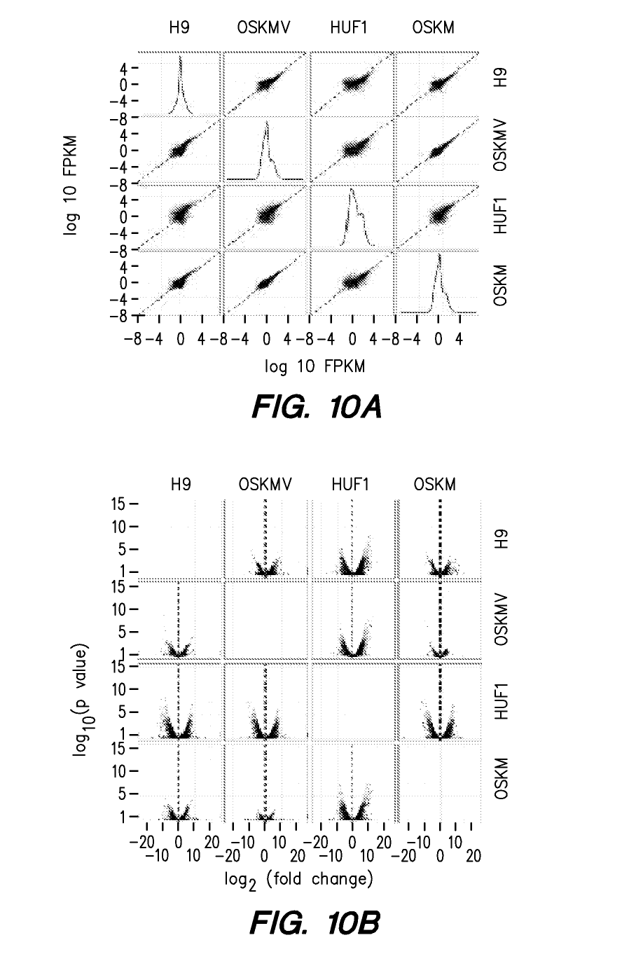

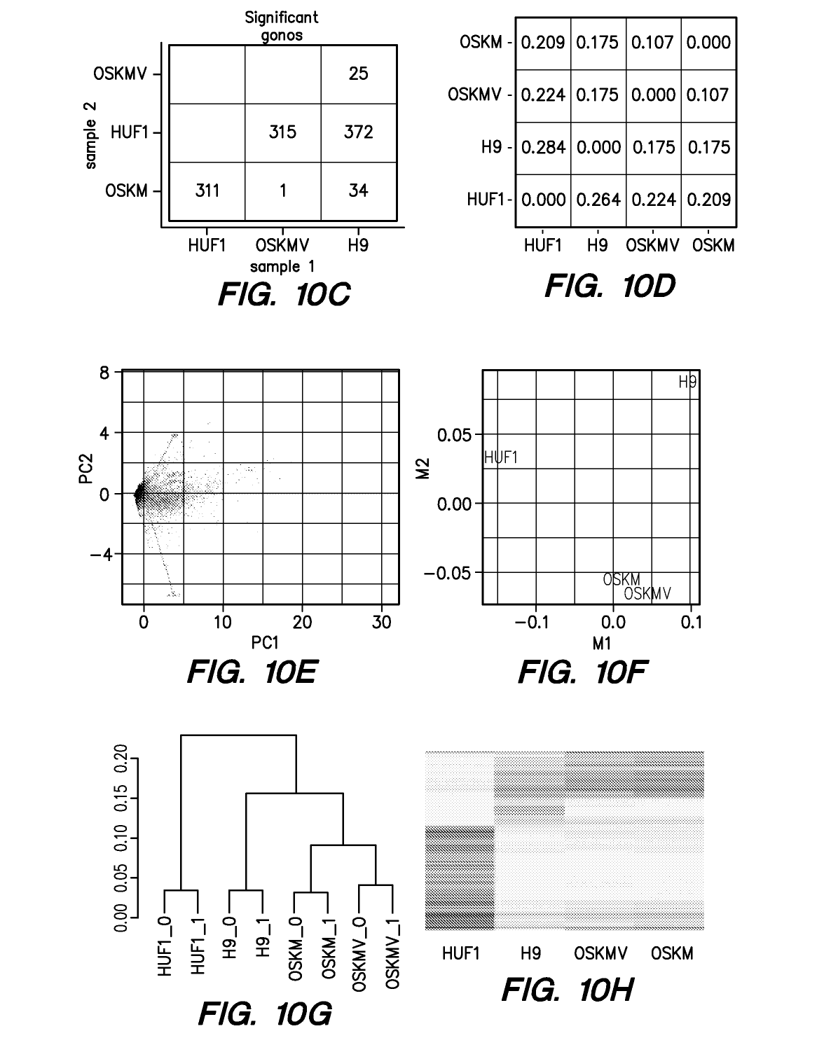

To examine global gene expression, we performed RNAseq followed by differential gene expression analysis on the OSKM and OSKMV derived HUF1 line together with controls (parental HUF1 and H9). As expected, gene expression profiles between HUF1 fibroblasts and the three pluripotent populations (OSKM, OSKMV, H9 hESCs) were significantly different (p<0.05) (FIG. 10). Interestingly, HUF1 fibroblasts and H9 hESCs expressed more genes at significantly different levels (p<0.05) than HUF1 fibroblasts compared to their RiPSC derivatives (FIG. 10c). Pairwise volcano and scatter plots emphasize the gene expression differences between each sample (FIG. 10a,b). We further explored the relationship between all four lines and visualized the Jensen-Shannon (JS) distances in a heatmap and a significant genes (p<0.05) overview matrix across all genes (FIG. 10c,d). Principal component analysis (PCA) and multi-dimensional scaling (MDS) revealed a very similar global gene expression in the OSKM and OSKMV derived lines that were significantly different (p<0.05) from the parental fibroblasts (FIG. 10e,f). Derived RiPSC lines also were distinct from the H9 hESC cell control but revealed a similar global gene expression phenotype (all three pluripotent lines with a positive second PC in PCA analysis). Hierarchical clustering analysis (FIG. 10g) and a heatmap of differentially expressed genes (FIG. 10h) highlighted the relationship between all pluripotent stem cell lines. When we compared both derived RiPSC lines with hESCs, we observed only a few genes, isoforms and transcription start sites (TSSs) with statistically significant differential expression, consistent with previous reports.

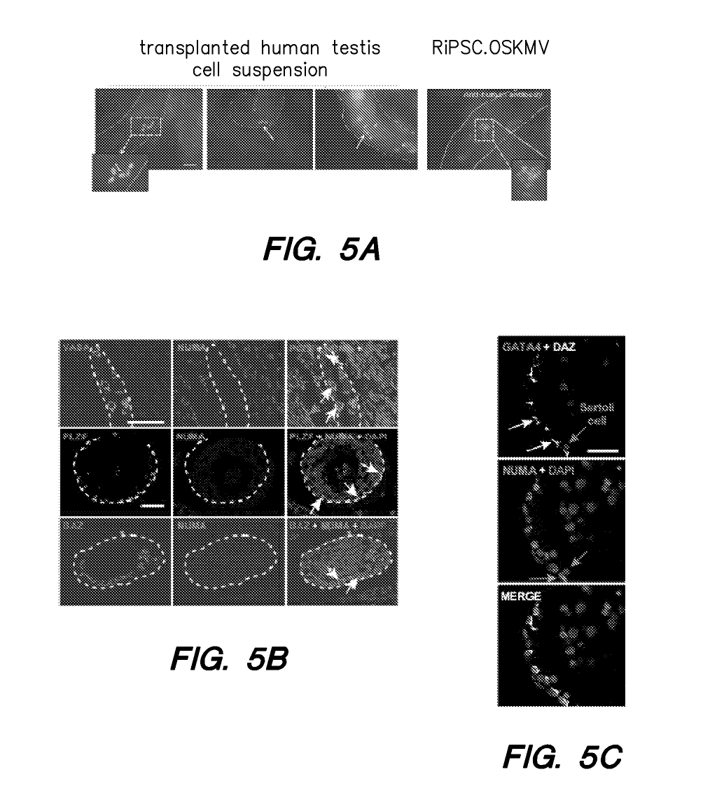

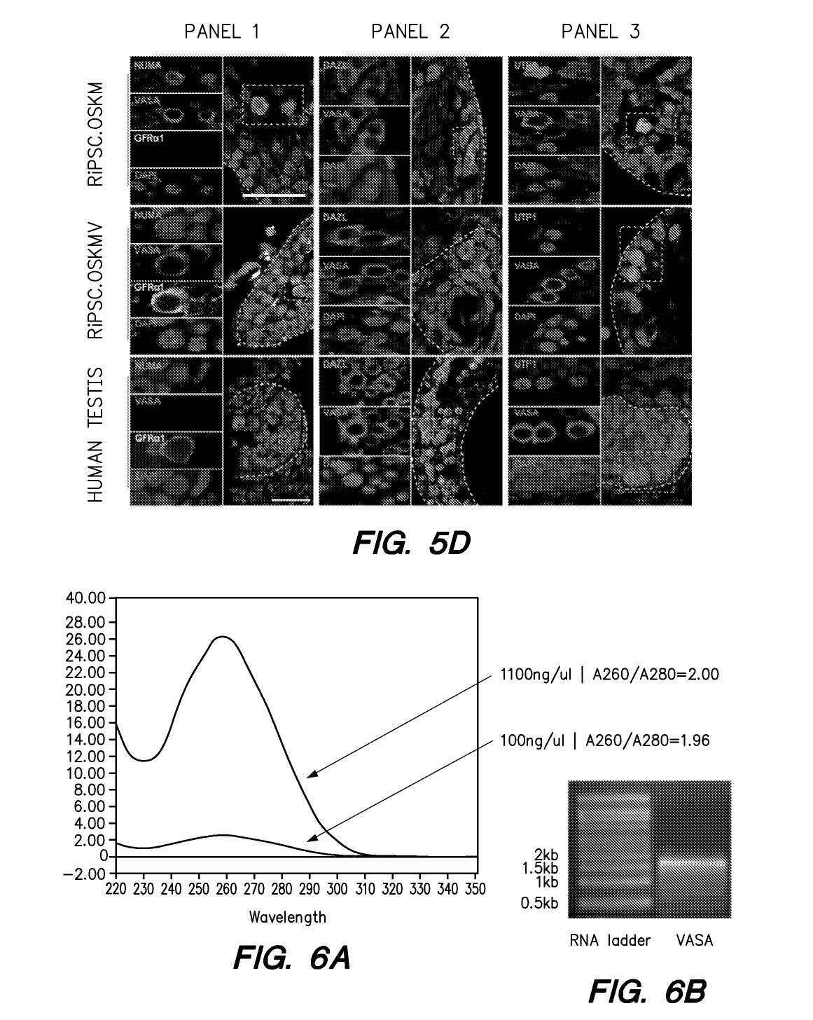

Xenotransplantation unveils germ cell formation in OSKM and OSKMV reprogrammed cells. Testicular assays of stem cell pluripotency typically depend upon injection of cells into the testicular capsule rather than into the tubules per se. In contrast, here we made use of a xenotransplant assay in which cells from reprogrammed lines were directly injected into the seminiferous tubules of busulfan-treated immune deficient nude mice as previously described. We used this assay to investigate OSKMV and OSKM cell differentiation into germ cells in vivo. Given the similarities of undifferentiated pluripotent ESCs and PGCs in both mouse and humans, we reasoned that OSKM and/or OSKMV cells might be directed to form germ cells with injection directly into mouse spermatogenic tubules. As previously reported, transplanted human spermatogonial stem cells migrate to the seminiferous tubule basement membrane and proliferate to form chains and patches of spermatogonia that persist long-term. Note that complete spermatogenesis is a function of evolutionary distance such that rat transplantation into mouse tubules or human transplantation into nonhuman primates is expected to yield complete spermatogenesis whereas human transplantation of germ cells into mouse tubules will be limited to formation of pre-spermatogonia, spermatogonia or and/or possibly early meiotic derivatives. As a positive control, we transplanted human fetal testicular cells (22 week old tissue) into busulfan-depleted spermatogenic tubules of immunodeficient mice and observed clusters and chains of spermatogonia two months post-transplantation (FIG. 5a). Transplantation of H9 hESCs (XX karyotype) served as a negative control that we hypothesized would result in formation of few, if any, germ cells in seminiferous tubules relative to our XY lines.

Two standard methods to examine the potential to form germ cells in transplants were pursued: Immunohistochemistry of serial sections of fixed tissue and whole mount staining of testis to assess potential differentiation to chains of spermatogonia, in vivo. Whole mounting is only possible when engraftment occurs in the absence of large masses post-transplantation. For immunohistochemical (IHC) analysis of serial cross-sections of transplanted tissues, we used a panel of well characterized germ cell markers.

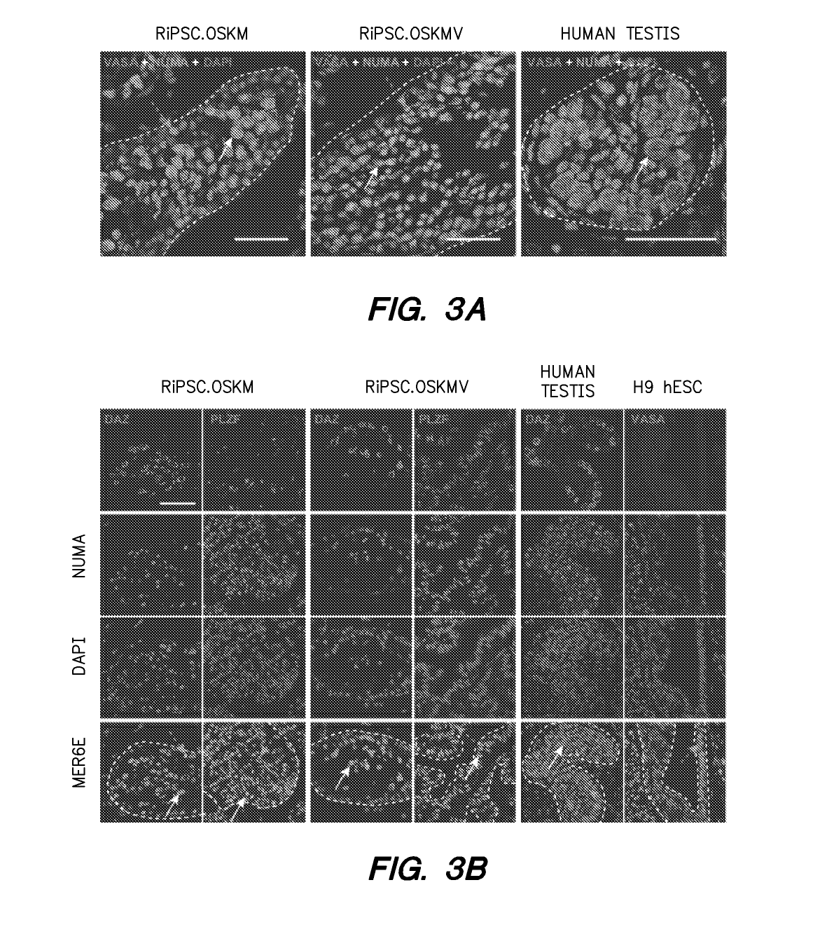

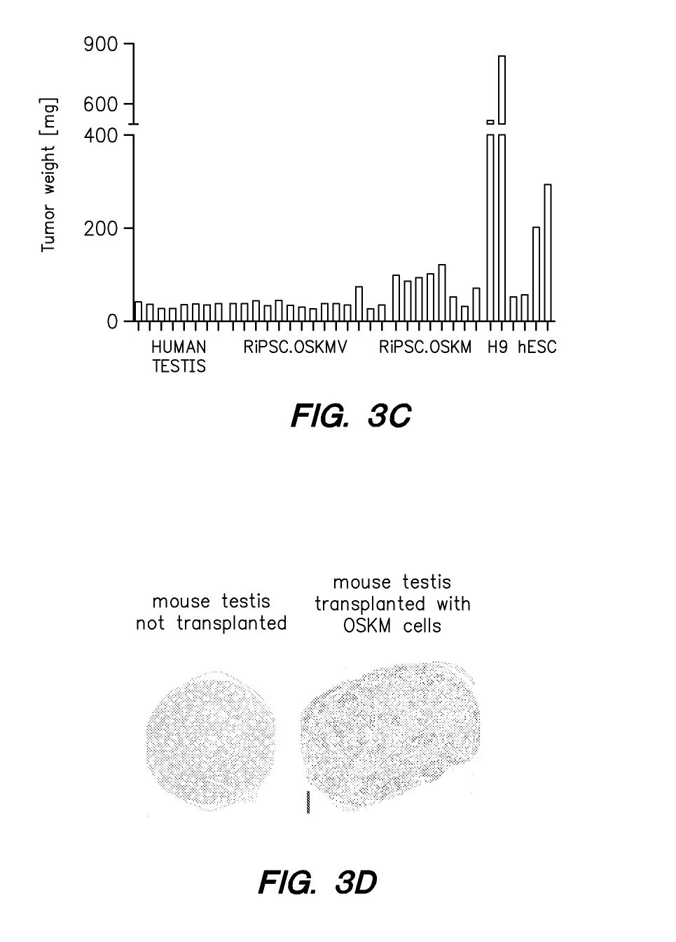

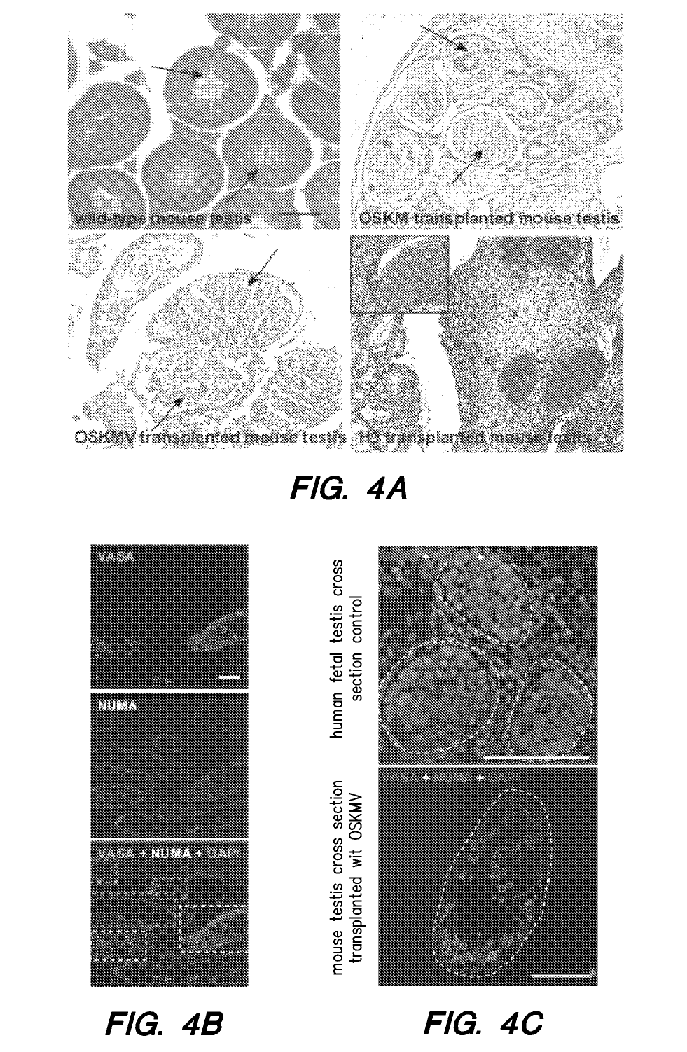

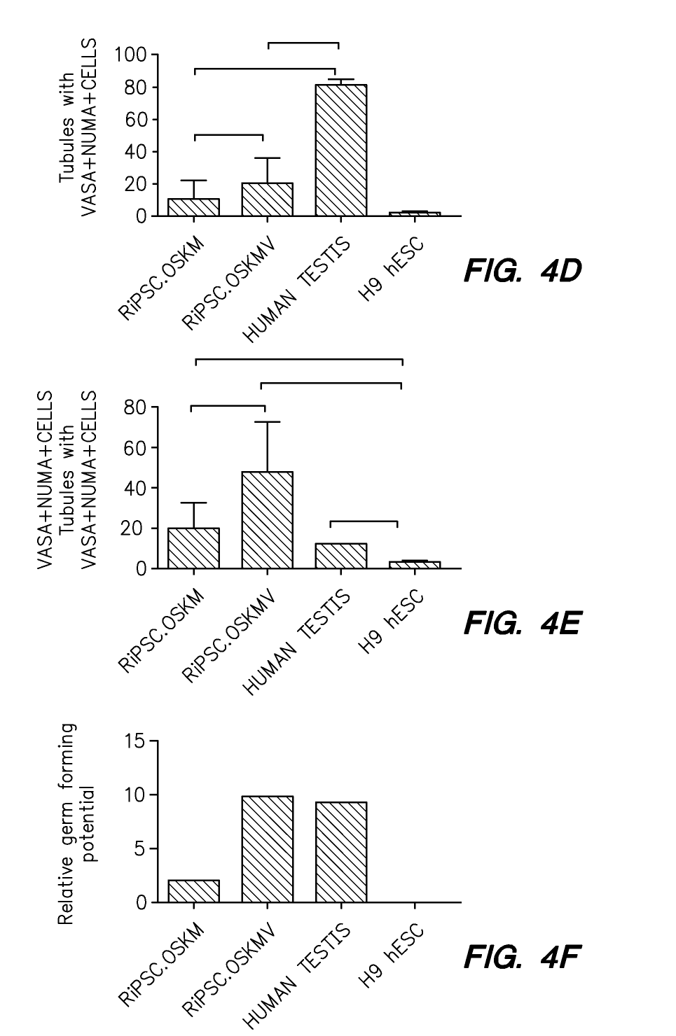

We injected undifferentiated RiPSCs derived with OSKM or OSKMV into spermatogenic tubules of 8 testes each to evaluate their potential to form spermatogonia in vivo in a transplant assay. Interestingly, depending on which factors were used during reprogramming, we obtained strikingly different results. Testes injected with OSKMV cells kept their naïve tissue structure 2 months post injection and 1 out of 8 testes was positive for a cluster of human cells that persisted long term (FIG. 5a). In contrast, testes transplanted with OSKM cells developed internal cell masses that appeared fibrotic with no signs of teratoma formation in all 8 out of 8 transplanted testes transforming the testis into an enlarged tissue (FIG. 3c,d). To examine outcomes more extensively, we then repeated the transplantation and used the same method of direct comparison of germ cell activity across transplantations. Our replicate transplantations consisted of OSKMV cell injections into the seminiferous tubules of an additional 6 testes with subsequent IHC analysis of serial cross sections regardless of tissue structure. We confirmed previous observations that indicated that OSKMV-derived iPSCs do not result in formation of large masses of cells but instead leave the mouse testis structure intact. Note that this is in contrast to testes transplanted with H9 (XX) control cells which developed enlarged tissues in the majority of testes similar to OSKM cells and in addition formed teratoma-like structures indicative of germ layer tissue formation (FIG. 4a).

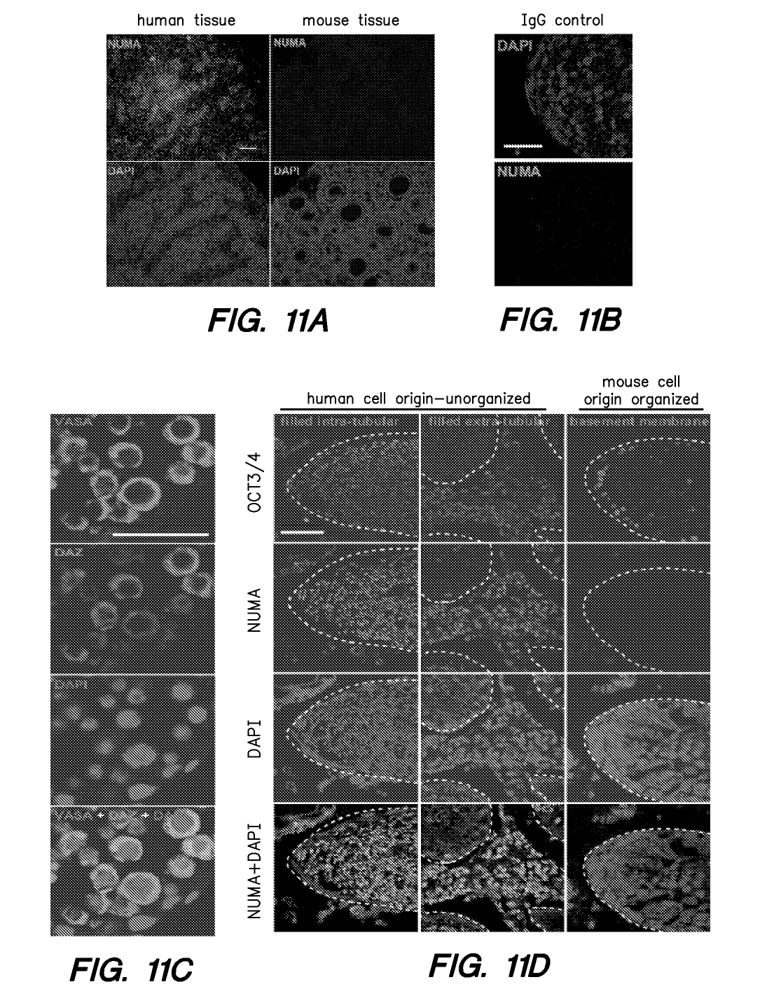

Next, we focused on IHC analysis and extensively screened serial cross sections for a panel of germ cell markers in all transplanted mouse testis (with OSKM, OSKMV, H9) and compared our results to cross sections from a human fetal testis sample. Our analysis revealed germ cells inside tubules of OSKM and OSKMV transplanted testes that are of human origin (nuclear NUMA, a human specific antibody, FIG. 11a,b) and co-localize with several germ cell specific markers including VASA, DAZ and PLZF (FIG. 3a,b). In contrast, transplanted H9 cells that were detected inside tubules did not stain positive for germ cell markers (FIG. 3b). Note, busulfan-depleted spermatogenic tubules treated bear the risk of incomplete germ cell pool erasure, thus we carefully discriminated positively stained germ cells that are of human or mouse origin (FIG. 5b) in all of our IHC analysis and subsequent quantification efforts.

OSKMV in contrast to OSKM reveals greater germ cell forming potential. In order to quantify our immunohistochemical analysis, we counted tubules that stained positive for VASA/NUMA cells to determine the average fraction of tubules that had residing human germ cell marker positive cells (FIG. 4b). We observed approximately 20% for both OSKM and OSKMV transplanted cells (FIG. 4c,d). In contrast, we observed over 80% of tubules filled with NUMA/VASA double positive cells in the human fetal testis cross sections. Note, these are non-transplanted cells but instead are naturally existing germ cells. We then considered only those tubules positive for NUMA/VASA activity and counted individual cells across all sections; results indicate a more than 2-fold difference between OSKM and OSKMV with the latter being superior (FIG. 4e). To compare germ cell production between OSKM and OSKMV derivations and our human fetal testis control, we calculated relative germ cell numbers by multiplying the percent occupied tubules by the number of cells per tubule and compared them to our positive control. Our conversion resulted in a 5-fold difference between OSKM compared to OSKMV and the human fetal testis, respectively (FIG. 4f). Note that mouse testes that were transplanted with H9 cells had significantly lower to no germ cell activity in our quantitative analysis.

In our efforts to further examine germ cells were induced from iPSCs in vivo, we extended our panel of germ cell markers and demonstrate co-localization of 2 germ cell markers (VASA/DAZL and VASA/UTF1) (FIG. 5d and FIG. 11c) for OSKM and OSKMV transplanted cells in conjunction with the positive control (FIG. 5d). In addition we observed, in rare cases, cells that stained positive for GFRα1/NUMA/VASA in human fetal testis sections and OSKMV transplanted mouse testis sections only, suggesting that OSKMV iPSCs are not only superior in germ cell formation to OSKM iPSCs in vitro but also in vivo.

To evaluate whether transplanted cells differentiated to other lineages (such as Sertoli cells) or remained in their undifferentiated state, we co-stained for NUMA and germ cell markers with GATA4, a Sertoli cell marker. We observed GATA4 positive cells (FIG. 5c) at the basement membrane of tubules that did not co-stain with NUMA, indicating their murine origin; we note that the GATA4 cells resided in the vicinity of NUMA/DAZ double positive cells highlighting their supportive phenotype in germ cell differentiation. Tubules filled with large numbers of cells both intra-tubular and extra-tubular appeared to be unorganized, of human origin and OCT3/4 positive, suggesting an excess of transplanted cells that retained their undifferentiated state (FIG. 11d). This is in contrast to OCT3/4 positive cells which did not co-localize with NUMA but instead revealed an organized staining pattern along the basement membrane indicating endogenous cell of mouse origin.

### DISCUSSION

In this study, we use in vitro transcribed modified mRNAs in combination with immunosuppression molecules and the appropriate culture conditions to reprogram somatic target cells into induced pluripotent stem cells in feeder- and xeno-free culture conditions. With this technology, we eliminate potential limitations of germ cell production for basic and clinical studies such as insufficient silencing of ectopic gene expression, random genomic integration associated with the risk of mutation, and low kinetics (2-4 weeks). Moreover, we included VASA mRNA during cellular reprogramming and demonstrate that similar efficiencies and kinetics were observed in terms of line derivation as with the Yamanaka factors alone. Moreover, in spite of the fact that they revealed an overall decreased pluripotent state based on gene expression analysis, they passed all in vitro as well as in vivo plurioptency assays. Yet, OSKMV lines differed primarily in two major ways, in particular: 1) Expression of early germ cell markers in vitro and 2) in phenotypes associated with xenotransplantation.

The relationship between germ cells and pluripotent stem cells (hESCs/iPSCs) is of particular interest but to date, little is known of molecular requirements for human germ cell specification. The finding that transient ectopic expression of VASA during reprogramming endows RiPSC clones with a PGC-like phenotype was further confirmed with BMP4 differentiation assays and the ability to initiate and enter meiosis. OKSMV derived clones respond with a significant upregulation (p<0.01) of the stage-specific germ cell markers NANOS3, VASA and DPPA3 upon BMP4 differentiation and a greater subset of punctuated and elongated SCP3 staining patterns were observed compared to the OSKM counterpart and control lines. These observations are complemented by studies of RNAseq that demonstrate that, on a global level, the transcriptional programs of OSKMV and OSKM derived RiPSCs, as well as epigenetic programs, are remarkably similar to each other and hESCs. Notably, PGCs are clearly related to pluripotent stem cells in many aspects, both on a molecular and cell functional level. PGC culture in vitro can result in derivation of pluripotent embryonic germ cell (EGC) lines capable of differentiating to somatic and germ line progeny cells; moreover, recent reports show evidence that the closest in vivo equivalent of an ESC is the early germ cell. Thus, it is tempting to speculate that with the inclusion of VASA, we can reprogram a somatic cell into a pluripotent cell type that is ‘primed’ to become a PGC in vitro and in vivo more efficiently than an embryonic stem cell or a OSKM factor induced iPSC.

Finally, we pursued the idea to differentiate undifferentiated iPSCs in vivo when directly injected into spermatogenic tubules. Transplantation of iPSCs in various organs has successfully been reported in different disease mouse models emphasizing their potential for cell therapy. We note that there are no previous reports of direct injection of human pluripotent stem cells into spermatogenic tubules; we hypothesized that OSKM and/or OSKMV cells would form spermatogonia in mouse testes with the latter perhaps being more efficient based on PGC-like resemblance in vitro. Notably, OSKM cells developed cell masses (distinct from teratomas in the lack of diverse cells of the three germ layers) in transplanted testis resulting in enlarged tissues that were subject to immunohistochemistry studies. Whole mount analysis was not performed due to the size and extent of tissue formation. A thorough analysis of germ cell activity in cross-sections of isolated cell masses however identified cells that migrated into the basal membrane of the mouse tubules and were positive for multiple germ line markers as well as the human specific marker NUMA. Interestingly, cells reprogrammed with OSKMV formed no cell masses in all injected sites (14 testes) but were able to colonize in vivo to form a small cluster in one whole-mount evaluated testes in this study. However, location and appearance cannot rule out that detected cells are simply a cluster of undifferentiated human iPSCs. Thus, we directly compared testes of transplanted OSKM vs OSKMV cells in IHC analysis and observed that OSKMV cells are superior in germ cell formation in vivo with higher frequency of cells expressing germ cell markers, inclusion of markers such as of GFRα1 and a reduction in exogenous masses of cells with characteristics of undifferentiated iPSCs. This correlates with our findings in in vitro differentiation assays. Based on these results we suggest that OSKMV cells and OSKM cells responded differently to the seminiferous tubule environment post transplantation.

Inclusion of VASA in mRNA-based derivation of iPSCs and techniques for transplantation provide a quantitative functional assay to characterize germ cell activity in derived iPSC populations. However, it is important to note limitations associated with cell loss and failure of donor cell foci to exhibit spermatogonial features as previously described. We suggest that the titration of number of cells transplanted may be critical to avoid “overbooking” of the somatic niche and/or creation of an artificial niche that promotes proliferation of iPSCs rather than differentiation.

In summary, coupling of mRNA reprogramming and transplantation holds great promise for fertility restoration and preservation in men. We note that the mRNA based reprogramming method is likely to be preferable for potential clinical applications with germ cells as risk of integration is absolutely minimized. Moreover, infertility is common, affecting 10-15% of couples with approximately 50% of infertility cases linked to a male factor. Previous studies with nonhuman primate models serve as a model for extension of results here. We propose that the transplantation of RiPSCs derived with OSKMV merits further investigation in nonhuman primate models towards the goal of clinical applications to begin the process of exploring methods to alleviate the devastating consequences to quality of life that are encountered by many infertile men.

### Materials and Methods

Cells. BJ human fibroblast cells (passage 6) were established from normal human fetal foreskin and purchased from Stemgent (Cambridge, Mass.). Human adult dermal fibroblasts were derived from a 28 year old healthy control male (HUF1), 30 year old healthy control female (HUF3), and a 31 year old female with premature ovarian failure (HUF9). To derive HUF lines, adult donors were consented and an inner arm 4 mm skin punch biopsy was obtained at the Stanford University Dermatology Clinic by a qualified dermatologist. GM13325 fibroblasts were purchased from Coriell.

Derivation of mRNA Induced Pluripotent Stem Cells (RiPSCs).

For feeder-dependent reprogramming, irradiated human foreskin fibroblasts (NuFFs, GlobalStem) were seeded at 250 k per well of a 6 well plate on 0.2% gelatin. Next day, 1×10₄-2.5×10₄ target fibroblasts per well were plated on top of the NuFFs and cultured in MEF media. 24 h after MEF media was changed with Pluriton media (Stemgent) supplemented with Pluriton supplement (Stemgent) and B18R (200 ng/ml, eBioscience). Cells were transferred to a low oxygen environment (5%) for higher reprogramming efficiencies before the first transfection. After 2 h of equilibration in low oxygen conditions mRNA cocktail containing OSKM(V)g (Oct3/4, Sox2, Klf4, cMyc, (VASA, d2eGFP) was transfected and repeated every 24 h until colony formation was observed. Incubation of mRNA and transfection mix with cells was carried out for 4 h. To compensate for the NuFF detachment, media was replaced with fresh NuFF-conditioned Pluriton media at day 6 post first-transfection (Stemgent) containing Pluriton supplement and B18R recombinant protein (200 ng/ml) to inhibit cellular immune response. Reprogramming was also performed in hES media which was replaced with NuFF conditioned hES media around day 6 post first-transfection.

For feeder-free reprogramming target fibroblasts were seeded at 1×10₄-4×10₄ cells per well of a 6 well plate on 0.2% gelatin coated wells and cultured in MEF media. 24 h later MEF media was replaced with NuFF-conditioned Pluriton media and the protocol for feeder-dependent reprogramming was followed. Primary colonies were picked onto fresh culture dishes coated with Matrigel (BD Bioscience) and media was replaced with mTeSR1 (STEMCELL Technologies) supplemented with 5×mTeSR1 supplement (STEMCELL Technologies). Established iPSCs lines were cultured under 20% oxygen conditions.

Gene Expression Analysis.

Gene expression analysis was performed using Real-Time PCR using a micro-fluidic platform (Fluidigm Corporation) analysis and following manufacturer's protocols. Briefly, purchased forward and reverse primer pairs were mixed together to a final concentration of 20 μM. All primer pairs were pooled together at a final concentration of 200 nM each for pre-amplification. CellsDirect 2× Reaction Mix (Invitrogen), SuperScript III RT Platinum Taq Mix (Invitrogen), 4× Primer Mix (200 nM) and TE buffer were prepared at total volume of 9 μl. Cells (10-200) were manually picked under the dissection hood added in 1 μl to each reaction and the following thermal cycling protocol was set: Reverse transcription—50° C., 15 min; inactivate RT/activate Taq—95° C., 2 min; 18 cycles—95° C., 15 sec and 60° C., 4 min; 4° C. hold. ExoSAP-IT treatment removed unincorporated primers and was performed at 37° C., 15 min (digest) and 80° C., 15 min (inactivation). Reaction was diluted 1:5 in TE buffer and stored at −20° C. or immediately used for Sample Pre-Mix. Sample pre-mix, samples and assay mix was prepared according to the protocol. The Fluidigm chip was primed and loaded with the assay and sample mix. Data was collected and analyzed using the Fluidigm Data Collection Software v.3.0.2.

Teratoma Formation.

For each graft, one RiPSCs line from one confluent 10 cm dish was enzymatically harvested, washed and resuspended in a 1.5 ml tube containing 300 μl 30% Matrigel diluted in PBS. Cells were then injected subcutaneously into female SCID mice (Charles River Laboratories International, Inc.). Visible tumors were dissected 4-8 weeks post transplantation and fixed overnight with 4% PFA diluted in PBS. Kidney capsule injection was performed as follows: Cells were prepared (manually picked in 100-200 cell clumps in 30 μl culture media) and the recipient mouse was anesthetized. The mouse was placed on its abdomen on a sterile paper towel. A vertical incision was made through the skin along the animal's spine, about 2 cm from the base of the tail to the top curve of the spine. With forceps the edge of the incision on one side was gripped and skin was carefully separated from the peritoneum by placing closed scissors under the skin and gently opening the scissors. A small incision into the peritoneum was made and the kidney was localized. With a little pressure on the abdomen, the kidney was exposed and popped out of the body cavity. A small tear with a sharp glass pipet was made in the kidney capsule and cells were inserted beneath the kidney capsule using a mouth pipet. The kidney was placed back into the body cavity, the skin was pulled upward and skin was clamped together using sterile surgical clamps. 3-4 weeks after the surgery tumors were dissected and fixed overnight in 4% PFA diluted in PBS. Fixed samples were sent to AML Laboratories (Baltimore, Md.) for paraffin embedding, sectioning and staining with hematoxylin and eosin. Sections were then examined for the presence of tissue representatives of all three germ layers.

Xenotransplantation Assay.

Human cell lines were transplanted into the testes of busulfan-treated, immune-deficient nude mice (NCr nu/nu; Taconic) as previously described for primate and human spermatogonia. Briefly, immunodeficient nude mice were treated with a single dose of busulfan (40 mg/kg, Sigma) at 6 weeks of age to eliminate endogenous spermatogenesis. Xenotransplantation was then performed 5 weeks after busulfan treatment by injecting cell suspensions containing 10% trypan blue (Invitrogen) into the seminiferous tubules of recipient testes via cannulation of the efferent ducts. Approximately 7 μl of cell suspension was injected per testis.

Eight weeks after transplantation, recipient mouse seminiferous tubules were dispersed gently with Collagenase IV (1 mg/mL) and DNase I (1 mg/mL) in D-PBS and fixed in 4% paraformaldehyde, as previously described. Clusters of human cells were observed on the basement membranes of recipient mouse seminiferous tubules by staining with a rabbit anti-primate testis cell primary antibody and a goat anti-rabbit IgG secondary antibody conjugated with AlexaFluor488 (Invitrogen). All dehydration, rehydration, and staining steps were carried out in 12-mm Transwell baskets (Corning Life Sciences) to prevent loss of seminiferous tubules.

Immunohistochemistry (IHC).

Formalin-fixed mouse testes from xenotransplants were sent to AML Laboratories (Baltimore, Md.) for paraffin embedding and sectioned into serial cross-sections of 10 mm thickness each. For immunostaining, testes sections were deparaffinized in xylene (thrice, 10 min each), rehydrated through a graded series of treatment with ethanol (100%, 90%, 80%, and 70%, 5 min each) and rinsed in tap water. For all samples, antigen retrieval was performed by boiling the sections in 0.01 M sodium citrate buffer (pH 6.0) for 20 min, followed by incubation at RT for 30 min. A 10% solution of normal donkey serum (Jackson ImmunoResearch) in PBS was used as a blocking buffer. Sections were incubated with the following primary antibodies diluted in blocking solution (0.25% BSA, 0.3% Triton X-100, sterile PBS) overnight at 4° C.: VASA (R&D Biosystems), NUMA (Abcam) and PLZF (Calbiochem), UTF1 (Millipore), and DAZ (Abcam). The sections were washed and immunofluorescence staining was performed by incubating sections with fluorescently labelled secondary antibodies raised in donkey. Stained sections were mounted in ProLong Gold Anti-fade mounting media containing DAPI (Life Technologies). Negative controls included incubation with Rabbit IgG antibodies and omission of the primary antibody for all samples. Quantification of stained sections for NUMA/VASA double staining was determined manually from 3-5 independent 10× fields taken from 3-5 different depths in testis tissue and from at least 3 separate biological replicates.

Statistical Analysis.

Analysis of variance and statistical comparisons were performed using GraphPad Prism (La Jolla, Calif.) and SPSS (IBM, Armonk, N.Y.) with statistical significance set at 0.05. Student's two-tailed t test was used to determine statistical significance for data generated from Fluidigm gene expression assays. Fisher's exact test was performed for bisulfite sequencing analysis.

Cell Culture.

Human fibroblast cell lines were cultured on 0.2% gelatin (Sigma) coated wells in DMEM-FBS a culture medium consisting of Dulbecco's modified Minimal Essential Medium+GlutaMAX (DMEM), 10% fetal bovine serum (FBS), 100 U/ml penicillin and 100 μg/ml streptomycin. When 80-90% confluent, cells were passaged using TrypLE Express and replated at a 1:3 dilution. Human ES cells (H9) and RiPSCs were cultured in hES media containing DMEM/F12 (Invitrogen) supplemented with 20% Knockout Serum Replacer, 2 mM L-glutamine, 0.1 mM Non-Essential Amino Acids (NEAA), 0.1 mM 2-Mercaptoethanol (Millipore) and 10 ng/ml b-FGF on irradiated mouse embryonic fibroblasts.

For feeder-independent maintenance of human ESCs and iPSCs, basal mTeSR 1 medium (STEMCELL Technologies) supplemented with 5×mTeSR1 supplement (STEMCELL Technologies) was used. Culture plates were pre-coated with growth factor reduced matrigel (BD Biosciences). Cells were passaged mechanically or enzymatically using 200 units/ml of Collagenase IV or Dispase (STEMCELL Technologies), washed and replated at a dilution of 1:2 to 1:5. Differentiated cells were removed and/or cleaned under a laminar flow dissection hood.

Cultures were maintained at 37° C. and 5% CO₂ and medium changed every other day for fibroblast lines and every day for hESC and RiPSC lines. Fibroblasts were frozen in 90% FBS (Invitrogen) and 10% dimethyl sulfoxide (DMSO, Sigma). hESC and RiPSC cells were frozen in Bambanker (Wako Chemicals). Tissue culture reagents were purchased from Invitrogen unless otherwise stated.

Alkaline Phosphatase Staining.

Alkaline phosphate (AP) staining was performed using the Vector Red Alkaline Phosphatase Substrate Kit I (Vector Laboratories) following the manufacturer's instructions.

Immunostaining of Live Cells.

StainAlive DyLight 488 anti-Human TRA-1-60/TRA-1-81 antibody (Stemgent) was diluted in fresh cell culture medium to a final concentration of 5 μg/ml. Old medium was aspirated and replaced with medium containing diluted antibodies. Cells were incubated for 30 min at 37° C. and 5% CO₂. Medium was aspirated and cells were washed gently 2× with cell culture medium. Fresh cell culture medium was added and cells were examined under a fluorescent microscope using the appropriate filters. Cells were kept in culture after examination.

Design and Construction of In Vitro Transcription (ivT) Templates.

A “backbone sequence” containing 5′ and 3′ UTR regions, T7 promoter and multiple cloning site was synthesized by DNA2.0 (Menlo Park, Calif.). ORF PCRs were amplified from different plasmids as follows: human OCT3/4, SOX2, KLF4, cMYC, and d2eGPF from Addgene; VASA from our group. PCR reactions were carried out with HiFi Hotstart (KAPA Biosystems, Woburn, Mass.). Specific primer pairs were synthesized by the Protein and Nucleic Acid Facility (PAN, Stanford University). Products on agarose gel were cut and purified using Qiaquick gel extraction kit (QIAGEN, Valencia, Calif.). ORF fragments and DNA2.0 vector containing the “backbone sequence” were digested with AgeI and NheI (New England Biolabs, Ipswich, Mass.) for 45 min at 37° C., followed by agarose gel verification and purification with Qiaquick gel extraction kit (QIAGEN). Enzyme treated ORFs and DNA2.0 vector were ligated for 2 h with a PCR cycle program (30 sec: 10° C., 30 sec: 30° C.) using a T4 ligase (New England Biolabs).

Transformation was carried out with One Shot®TOP10 Chemically Competent E. coli (Invitrogen) according to the manufacturer's instructions and positive clone selection was verified with a Qiaquick Mini-Prep purification kit (QIAGEN) followed by a test digestion with SpeI (New England Biolabs). Plasmid inserts were excised by restriction digest and purified using Qiaquick PCR purification kit (QIAGEN) before being subject to template tail PCR. Vectors from Addgene containing the Yamanaka factors were used in addition to our own designed mRNA. A polyA tail was added with a T₁₂₀-heeled reverse primer and carried out with HiFi Hotstart (KAPA Biosystems) PCR reaction. Purification of amplicons was carried out with Qiaquick PCR purification kit (QIAGEN) and amplicons were used for ivT reactions.

In Vitro Transcription.

Synthesis for modified mRNA was carried out with the MEGAscript T7 kit (Ambion) according to the manufacturer's instructions with slight modifications. A custom ribonucleoside blend was used comprising 6 mM 3′-O-Me-m7G(5′)ppp(5′)G ARCA cap analog (New England Biolabs), 7.5 mM of adenosine triphosphate and 1.5 mM of guanosine triphosphate (Ambion), 7.5 mM of 5-methylcytidine triphosphate and pseudouridine triphosphate (TriLink Biotechnologies). Reactions were incubated for 3 h at 37° C., followed by DNase treatment for 15 min at 37° C. DNase treated RNA was purified using the MEGAclear kit (Ambion). Correct RNA synthesis and RNA purification was verified and quantitated using a Nanodrop (A260/A280 between 1.8-2.1) and concentration was adjusted to 100 ng/ml. RNA reprogramming cocktails were prepared by pooling individual 100 ng/μl RNA stocks to produce a 100 ng/μl total blend. Stocks were stored at −80° C. RNAse mediated RNA degradation was prevented by cleaning the working space and instruments with RNaseZap (Ambion).

Denaturing Formaldehyde-Agarose Gel Electrophoresis.

mRNAs were analyzed to verify specificity of ivT reaction and correct size of the transcripts. A 1.5% denaturing formaldehyde-agarose gel was prepared by dissolving 0.75 g agarose in 36 ml DI water, 5 ml 10×MOPS running buffer (Ambion) and 9 ml 37% formaldehyde (12.3 M, Sigma-Aldrich). mRNA samples (1 μg) were mixed with 3× the volume of Formaldehyde Loading Dye (Ambion) and 0.25 μl ethidium bromide (10 mg/ml, Bio-Rad) followed by heat denaturation for 15 min at 70° C. RNA ladder (RNA Millenium marker, Ambion) was treated like mRNA samples and used for size comparison.

mRNA Transfection.

mRNA transfection was carried out with RNAiMAX (Invitrogen) according to the manufacturer's instruction in a 6 well format. Briefly, RNA and reagent were first diluted in Opti-MEM basal medium (Invitrogen). 1.2 μg (100 ng/μl) RNA was diluted in 48 μl Opti-MEM and 6 μl RNAiMAX was diluted in 54 μl Opti-MEM. Both dilutions were combined to a total of 120 μl, briefly vortexed and incubated for 15 min at room temperature. After complex formation, mix was dispensed drop-wise into the culture medium. RNA transfection was performed in Pluriton media (Stemgent) supplemented with Pluriton supplement (Stemgent) and B18R interferon inhibitor (eBioscience) at 200 ng/ml or hESC media.

EB formation, in vitro differentiation and cardiomyocyte formation. RiPSCs cells from one confluent 10 cm dish were harvested, washed and seeded into 2 wells of an ultra low attachment plates (Corning) containing DMEM+20% FBS. 7 days after cells grew in suspension, embryoid bodies were transferred to gelatin-coated wells of a 24 well plate containing the same medium to allow the cells to attach. Medium was changed every 2-3 days for up to 2 weeks. Beating cardiomyocytes were observed 10-14 days after transfer to gelatin-coated plates. Cells were fixed in 4% PFA for staining of representative germ layer markers.

Differentiation Towards Primordial Germ Cells.

The developmental potential for in vitro PGC formation was assessed by treating cell cultures with BMP4 (R&D Systems, 50 ng/ml) or vehicle (4 mM HCl+0.1% BSA) in culture media. Samples were isolated at day 0, 2, and 4 and gene expression analysis was performed using Fluidigm.

Bisulfite sequencing. Genomic DNA was extracted using QIAamp DNA Mini kit (Qiagen) and processed using the Epitect Bisulfite Kit (Qiagen) according to manufacturer's instructions. Converted DNA was amplified by polymerase chain reaction (PCR) using primers published previously. PCR conditions were 95° C. for 10 min and 40 cycles of 95° C. for 1 min, 58° C. for 1 min, and 72° C. for 1 min, followed by 10 min at 72° C. (see. PCR products were purified with QIAquick PCR purification kit (Qiagen), cloned using TOPO TA cloning kit (Invitrogen) and the resulting plasmids transformed into OneShot® Top10 chemically competent E. coli (Invitrogen). 10 bacterial clones per genomic region (for pluripotency genes) and 20 bacterial clones per genomic region (for imprinted genes) were either sent to Sequetech for sequencing or picked and DNA was extracted using QIAprep Spin Miniprep kit (Qiagen) followed by reverse-sequencing using the M13 reverse primer (PAN facility Stanford University). Sequenced clones were aligned by Geneious software (Biomatters, Auckland, New Zealand) and CpG methylation analysis was performed by BioQ Analyzer software (Max Planck Institut Informatik, Saarbruecken, Germany).

Meiotic spreads. Differentiated cells were harvested in PBS and put on a glass slide dipped in a solution of 1% PFA in DW (pH 9.2) containing 0.15% Triton X-100 and 3 mM dithiothreitol (Sigma-Aldrich). The slide was then incubated overnight in 4° C. After fixation, the slide was washed in 0.4% Photoflo (Kodak) in DW and dried for 30 min at room temperature. Subsequent immunostaining was performed as follows. The samples were washed with PBS containing 0.1% Tween-20 (PBST) and treated with 0.5% Triton for 15 min. After washing with PBST, they were incubated in 1% BSA in PBS overnight at 4° C. The samples were incubated with a primary antibody diluted in 1% BSA/PBS, anti-Scp3 ( 1/400, Novus Biologicals) and anti-Cenpa ( 1/400, Abcam) for 3 h at room temperature. After washing with 1% BSA/PBS, the cells were incubated with a 1:1000 dilution of A488-conjugated anti-mouse IgG (Invitrogen) and A594-conjugated antirabbit IgG (Invitrogen) for 1 hr. After brief washing in 1% BSA/PBS the samples were then mounted on a glass slide in Vectashield anti-bleaching solution (Vector Laboratories, Burlingame, Calif.) containing 3 μg/mL 4′,6-diamidino-2-phenylindole (DAPI) followed by fluorescence microscopy.

RNA Seq.

Total RNA was extracted with the RNeasy Mini Kit (Qiagen) per manufacturer's instructions and subjected to cDNA synthesis. 100 ng of total RNA was subjected to first and second cDNA synthesis using the Ovation RNA-Seq System V2 (NuGEN Technologies, Inc.; San Carlos, Calif.) following the fragmentation with an average size of 200-300 bases using the Covars S-Series System. Briefly, 1-5 ug of each cDNA sample was diluted in 120 μl 1×TE buffer. The Covaris S-Series System settings were as follows: duty cycle—10%, intensity—5, cycles/burst—100, time—5 min. Illumina library construction was then performed using the NEBNext DNA Sample Prep Master Mix Set 1 and Agencourt AMPure XB beads for clean-up. Briefly, 10-25 ng of fragmented DNA was end repaired following the manufacturer's instructions of the NEBNext DNA Sample Prep master Mix Set 1 kit followed by cleanup with the Agencourt RNACIean XB beads. End repaired DNA was subject to dA-tailing followed by a second clean up.

dA-tailed DNA and adaptor were ligated followed by a PCR for enrichment of adaptor ligated DNA (98° C. for 30 sec; 17×98° C. for 10 sec, 65° C. for 30 sec, 72° C. for 30 sec; 72° C. for 5 min, 4° C. on hold). Samples were cleaned once again and the built library was analyzed on a HS Agilent DNA chip using a Bioanalyzer. Samples were sequenced in 2 lanes of Illumina HiSeq 2000 platform (Illumina, Inc) as 100 bp paired end reads. Quality check of raw data was processed through the web-based Galaxy platform using the FASTQC tool. Reads with a median score lower than 20 were trimmed using FASTQ Trimmer. Reads were then mapped using TopHat v.2.0.5 with default settings. The mean insert sizes as determined by the Bioanalyzer were employed in the TopHat mapping. Transcript assembly and expression level quantification of transfrags was performed using Cufflinks v.2.0.2 to filter out background and artifactual transfrags. Each sample was assembled individually and all assemblies were merged together using Cuffmerge. Transcripts with a p<0.05 were considered to be differentially expressed. Visualization of differential gene expression analysis was performed with CummeRbund v.1.2.0.

### Example 2

### Fate of iPSCs, Derived from Azoospermic Infertile Men, Following Transplantation to Murine Seminiferous Tubules

Much progress has been made in recent years in elucidating the molecular genetic requirements of germline formation and differentiation in diverse organisms, including studies focused on murine germline development in vivo and in vitro from mouse embryonic stem cells (mESCs). Studies indicate that a key set of transcriptional regulators, including Prdm1, Prdm14, and Tfap2c, comprises a tripartite transcriptional core that functions in suppression of somatic fate and acquisition of germline fate in vivo. In vitro studies have similarly demonstrated that induced expression of these factors converts mouse epiblast-like cells (mEpiLCs) to primordial germ-cell-like cells (PGCLCs); moreover, Prdm14 appears to be sufficient for this activity. The resulting PGCLCs are capable of contributing to spermatogenesis and fertile offspring following transplantation to murine seminiferous tubules. In parallel, in other studies, EpiSCs were shown to have an infinite capacity for generating PGCLCs as long as conditions were maintained to sustain pluripotency and self-renewal in vitro.

In contrast to mouse germline development, much less is known of the genetic and molecular requirements to establish the population of PGCs that ultimately gives rise to human sperm and oocytes. Indeed, this is in spite of the fact that infertility is remarkably common, affecting 10%-15% of couples. Moreover, genetic causes of infertility are surprisingly prevalent among men, most commonly due to the de novo deletion of one or more AZF (azoospermia factor) regions of the human Y chromosome. Pure sterile phenotypes in men with deletions vary from the complete absence of germ cells and sperm (termed Sertoli cell-only [SCO] syndrome) to production of germ cells that arrest in development (early maturation arrest; EMA) to very low sperm counts (oligospermia). It is not known whether the genes of the Y chromosome AZF regions are required for PGC formation, maintenance of germline stem cell populations, and/or commitment to later stages of meiosis and haploid germ cell morphogenesis. Because of the unique nature of Y chromosome gene content in men, studies that probe the function of genes that map to the AZF regions must be conducted on a human genome background.

In order to recapitulate human germ cell formation in vivo, we tested the hypothesis that the somatic niche of murine seminiferous tubules can direct formation and maintenance of GCLCs from undifferentiated human induced pluripotent stem cells (iPSCs). Thus, we generated patient-specific iPSCs from infertile men that harbor the most common genetic deletions of the AZF regions and then we induced germ cell formation from these iPSCs via xenotransplantation. Our studies offer a strategy for probing the function of naturally occurring mutations in germ cell development.

### Results

Derivation and Characterization of iPSCs from Azoospermic Men with Y Chromosome Deletions.

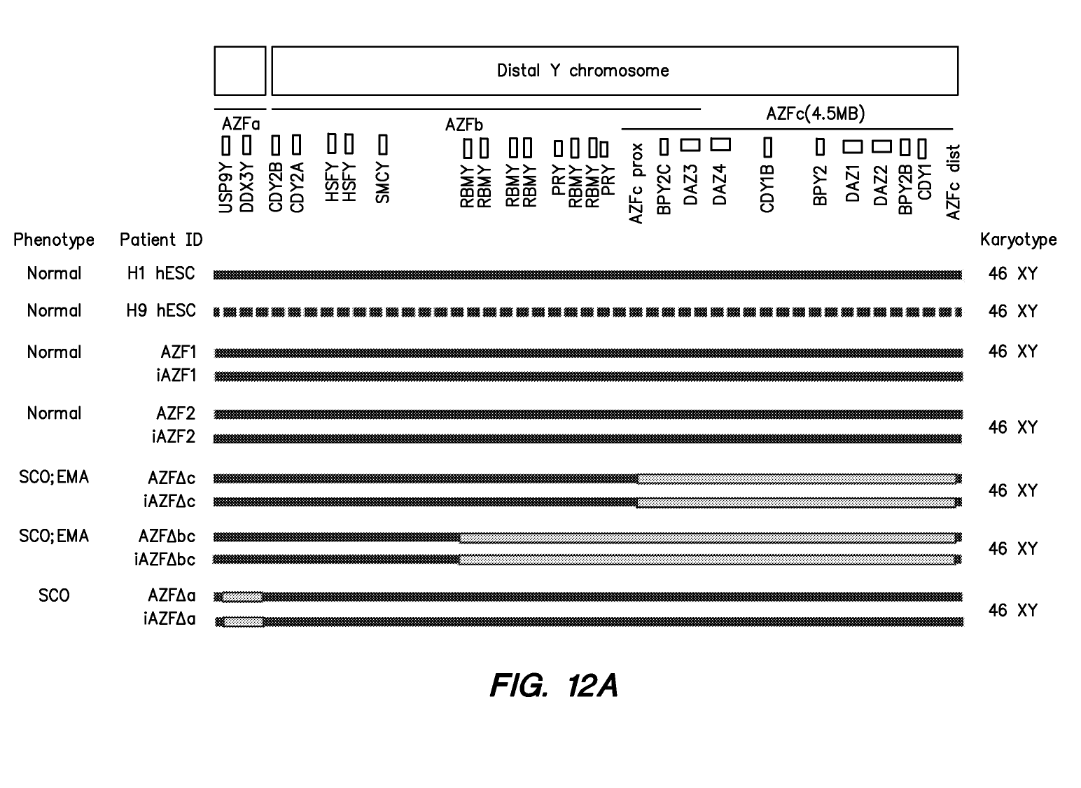

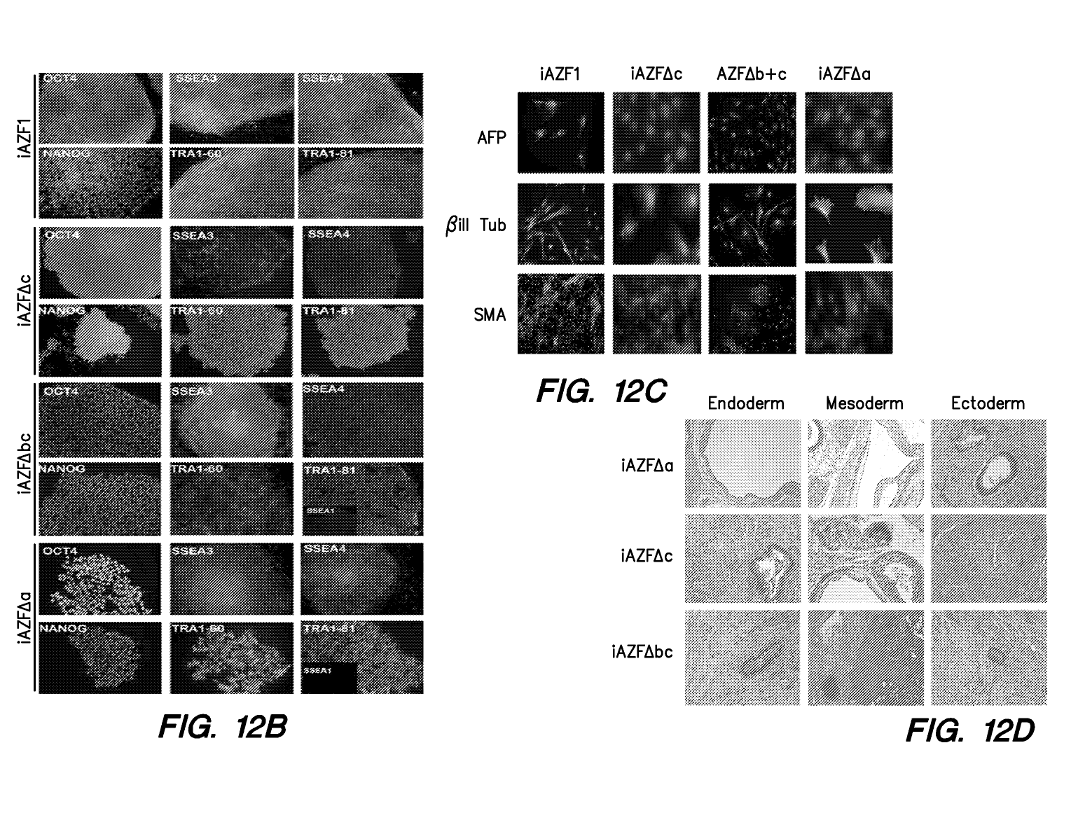

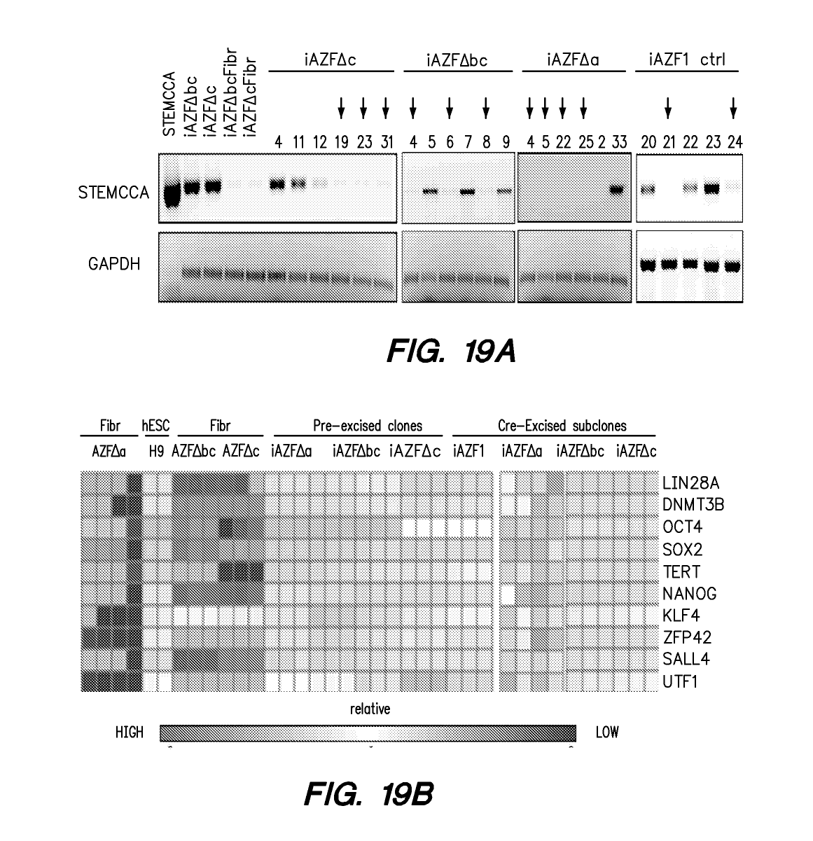

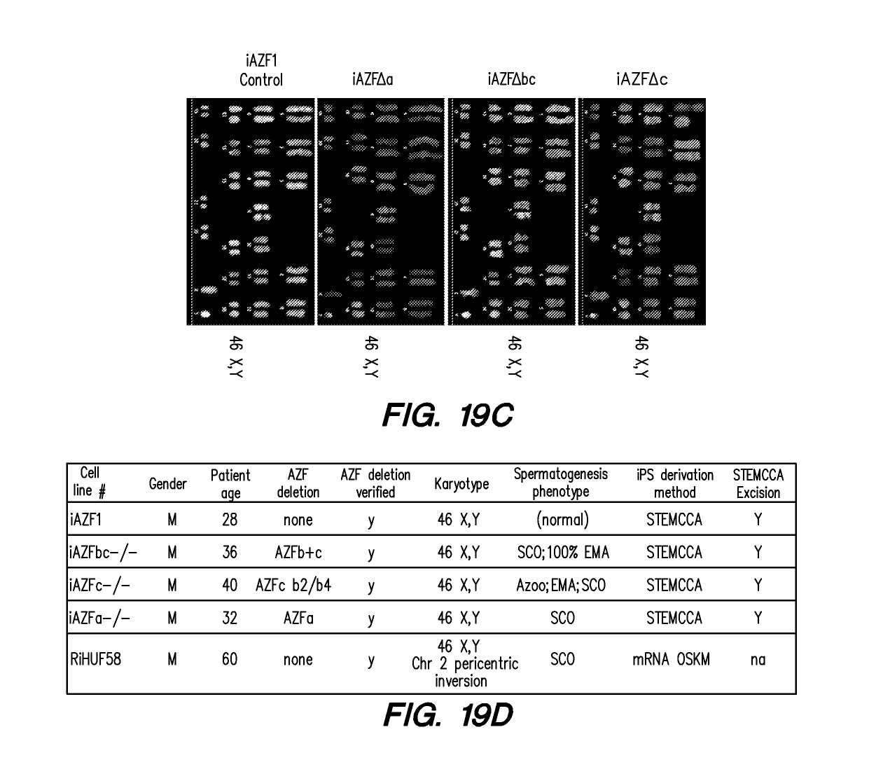

iPSC lines were derived from dermal fibroblasts from five males; lines were analyzed for Y chromosome deletions, and a deletion map was constructed (FIG. 12). We verified that the fertile controls (AZF1 and AZF2) had intact Y chromosomes, whereas all three infertile patients had deletions: one had a complete deletion of the AZFa region (AZFΔa); the second, a deletion of both the AZFb and AZFc regions (AZFΔbc); and the third, an AZFc deletion (AZFΔc). The men with AZFΔa and AZFΔc deletions presented with SCO syndrome and had no germ cells found in their testes upon extensive clinical examination; AZFΔbc was severely oligospermic. iPSCs generated from fibroblast cell lines (iAZF1, iAZF2, iAZFΔa, iAZFΔbc, and iAZFΔc) were isogenic with the parental fibroblast samples; no additional deletions occurred as a consequence of reprogramming. All iPSC lines met classic criteria of pluripotency (FIG. 12). They expressed pluripotency markers at the mRNA and protein levels (FIGS. 19B and 12B), were karyotypically normal (FIG. 19C; except for the presence of Y chromosome deletions as indicated), and differentiated in vitro and in vivo to cells of all three germ layers (FIGS. 12C and 12D).

In Vitro Differentiation of PGCs from Azoospermic Men and Controls.

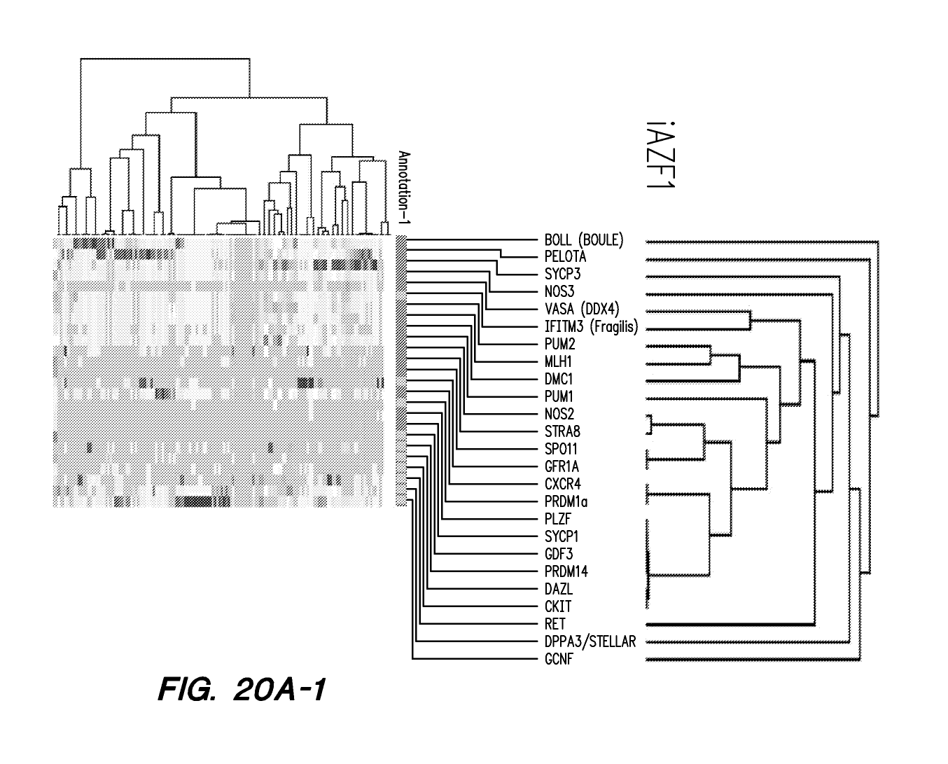

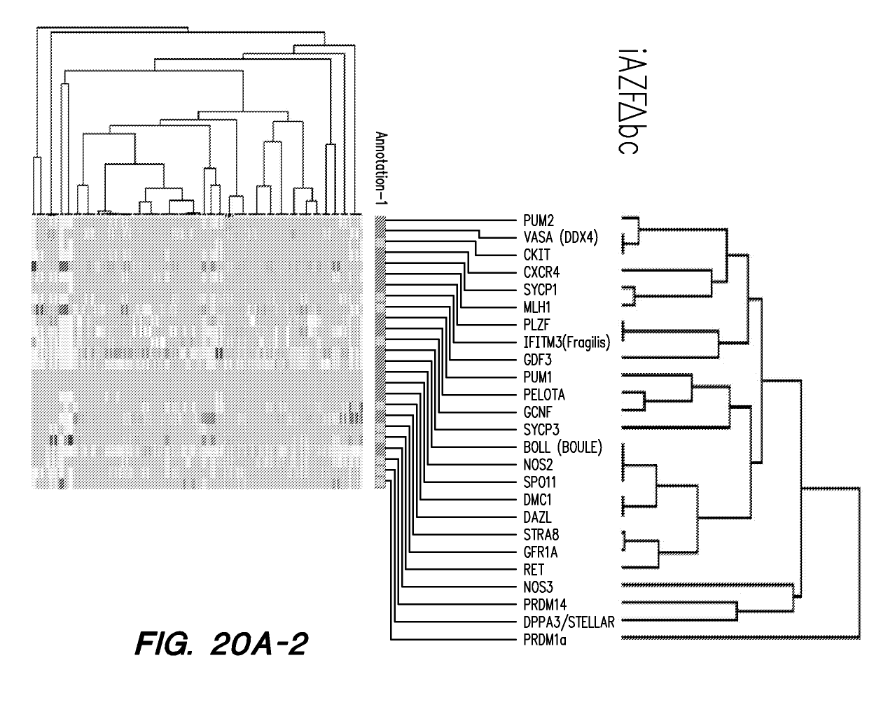

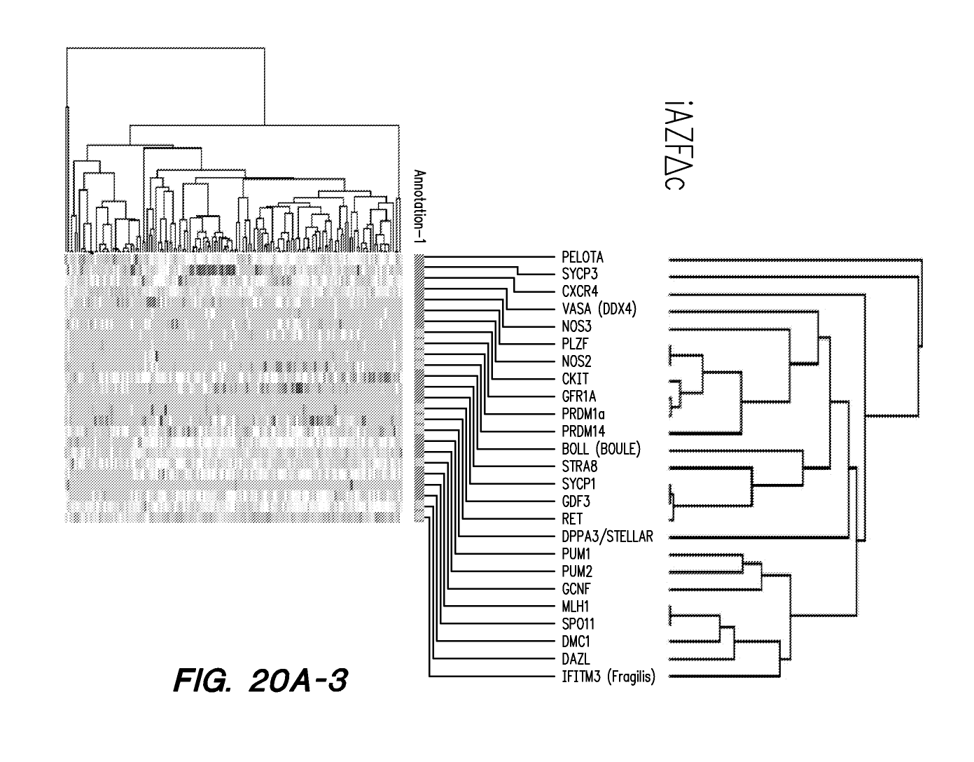

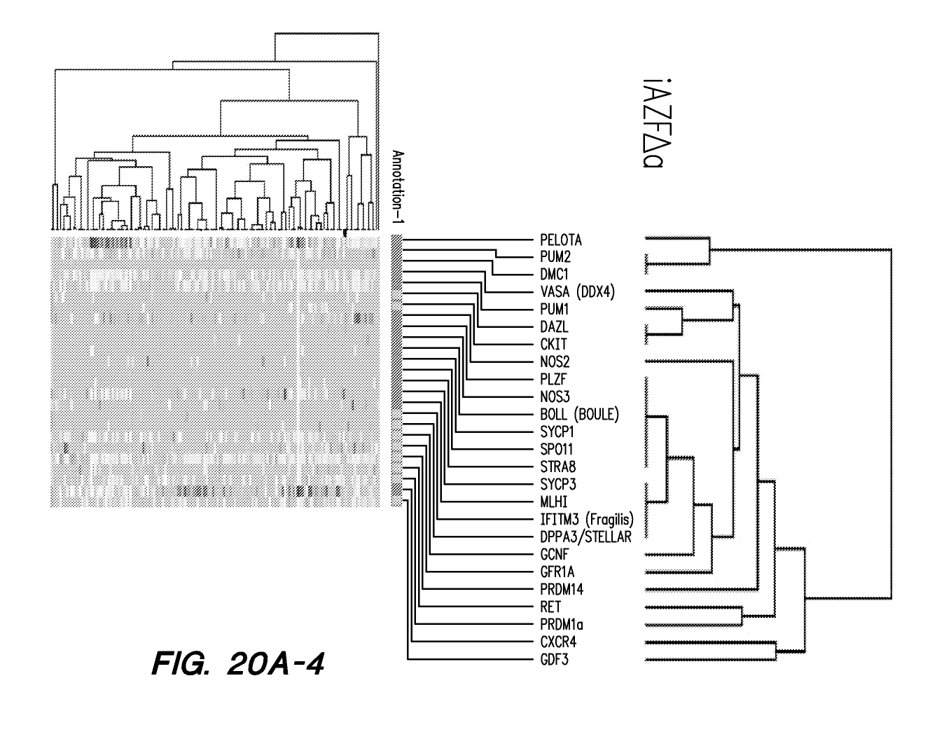

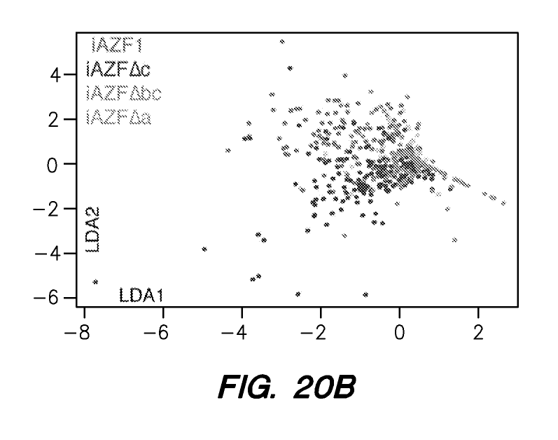

We previously reported germ cell differentiation from both hESCs and iPSCs in vitro. To assess germ cell development from azoospermic men relative to controls, we introduced a VASA:GFP reporter into all iPSCs, differentiated cells, and enriched for presumptive VASA:GFP+ single-germ cells via FACS (fluorescence-activated cell sorting). We observed that the percentage of GFP+ cells across all lines was similar (2%-8%) as determined from two independent clones per line (FIGS. 20A-20E) but that germ cells differed in gene expression as a function of genotype. First, to determine similarities and differences between single cells differentiated from each of the four cell lines, we performed hierarchical clustering on single-cell expression (150-160 cells) and constructed a condensed heatmap of 25 single cells (iPGCs) expressing VASA and GFP mRNAs (FIG. 20F). To evaluate germ-cell-specific gene expression of in-vitro-derived PGCs (iPGCs) from each line, we validated the identity of 50 individual cells that expressed the GFP transcript (13A) and queried expression of germ cell genes in a subset of cells for each line. Downregulation of key pluripotency genes and expression of VASA and other classical human PGC genes such as DAZL is considered indicative of a distinct PGC fate of iPSCs and hESCs following germ cell differentiation in vitro.

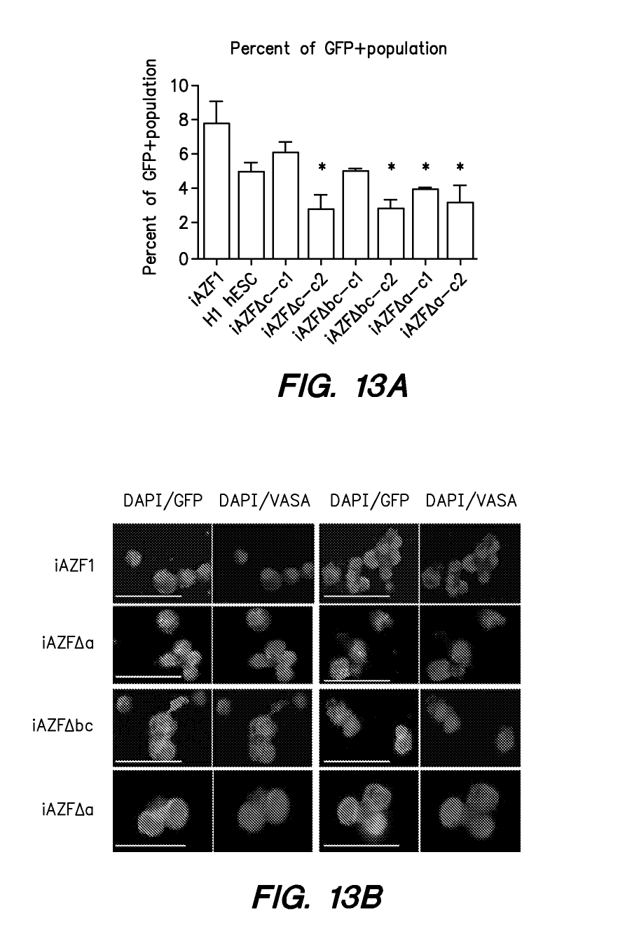

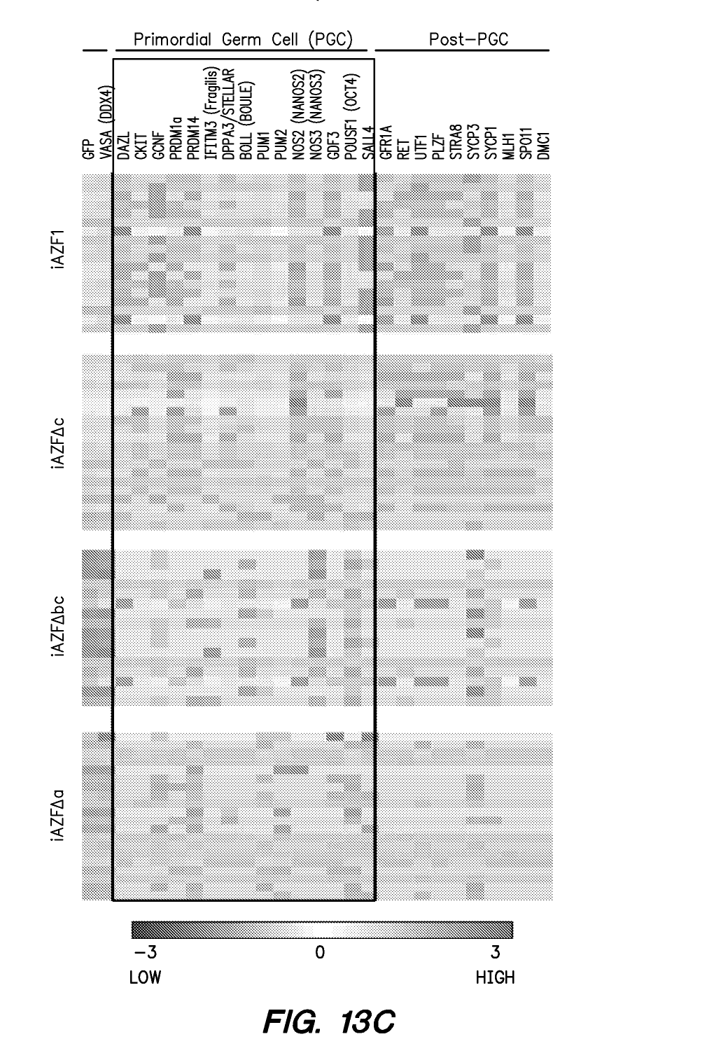

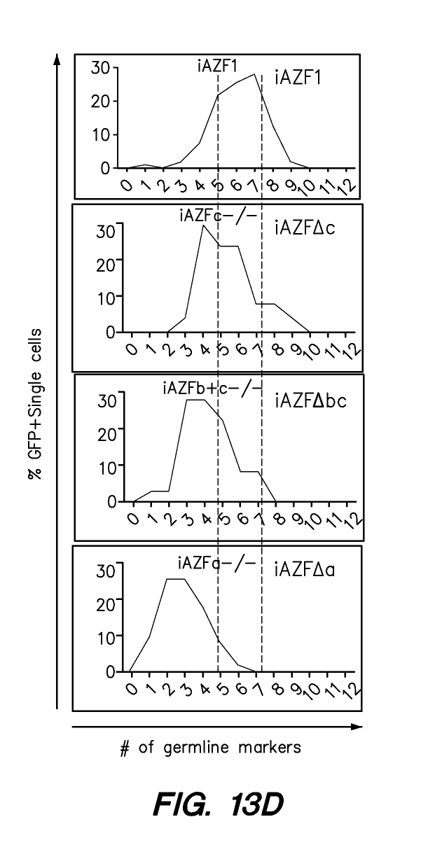

We observed that iPGCs derived from all five lines expressed PRDM1, PRDM14, and DAZL mRNAs (FIG. 13A). This was also reflected at the protein level for DAZL in GFP+ cells (FIG. 13B). However, the overall number of cells expressing VASA, STELLA, IFITM3, and NANOS3 genes varied across the lines, with the greatest number observed in iAZF1-derived iPGCs compared to AZF-deleted iPGCs (FIG. 13A). Moreover, we noted that GFP+ cell populations derived from iAZFΔa and iAZFΔbc lines contained the fewest number of cells expressing at least seven germ-cell-specific genes. At the protein level, iPGCs expressed VASA, STELLA, and DAZL, but we detected fewer cells positive for each protein in iAZFΔc, iAZFΔbc, and iAZFΔa cells relative to controls (FIG. 13B). For all iPGCs derived, core pluripotency genes were expressed at very low levels relative to undifferentiated cells (FIG. 13A, right). Moreover, linear discrimination analysis of 12 candidate germ cell genes in each population of GFP+ single cells revealed that iAZF1 and iAZFΔc iPGCs were distinct from iAZFΔbc and iAZFΔa iPGCs (FIG. 13C). To further quantify these observations, we determined the percentage of VASA+/GFP+ cells expressing between 1 and 12 candidate germ cell genes and observed that iAZF1 GFP+ cells expressed a minimum of 5-8 germ cell genes in 70% of the cell population (FIG. 13D, left column, dashed lines). By comparison, 70% of GFP+ single cells from each of the three AZF-deleted lines expressed a range of only one to six germ cell genes with the iAZFΔa and iAZFΔbc lines expressing the fewest germ-cell-specific genes within single cells. Further analysis of stage-specific gene expression revealed that, independent of genotype, the majority of GFP+iPGCs expressed at least four to six PGC markers (FIG. 13D, right column). In contrast, AZF-deleted germ cells, particularly from iAZFΔbc and iAZFΔa, expressed zero to two spermatogonial markers, whereas iAZF1 expressed two to four spermatogonial genes (FIG. 13D). On the basis of germ-cell-specific gene expression at single-cell resolution, these analyses suggest that all lines can differentiate to iPGCs regardless of genetic background. However, when iAZFΔa and iAZFΔbc cells are differentiated, fewer germ cells are produced relative to control iAZF1 cells.

Transplantation and Survival of Fetal Germ Cells to Murine Seminiferous Tubules.

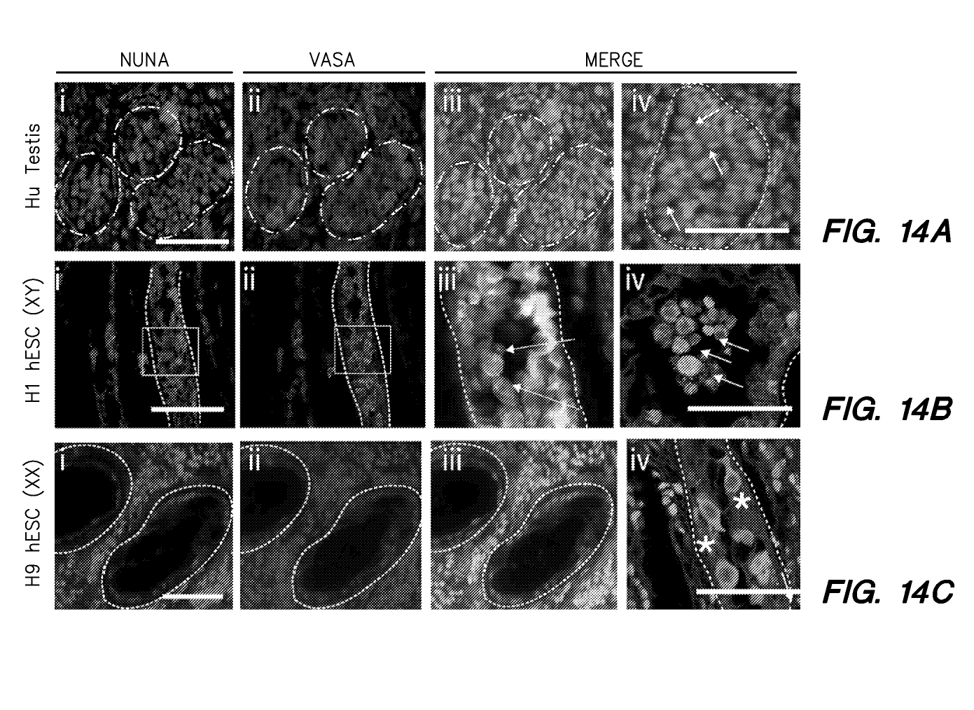

The gold standard for phenotypic characterization of stem cells is assessment of function in vivo. As previously reported, in transplantation with human spermatogonial stem cells, germ cells migrate to the seminiferous tubule basement membrane and proliferate to form chains and patches of spermatogonia that persist long term but do not appear to initiate or complete meiosis. To date, the fate of human pluripotent stem cells in the mouse seminiferous tubule has not been explored. To verify that human germ cells could survive and engraft inside murine seminiferous tubules, we transplanted human fetal testicular cell suspensions (22 weeks old) into busulfan-treated testes of immunodeficient mice (FIG. 14A). The use of busulfan treatment eliminates endogenous mouse germ cells from the seminiferous tubules (FIG. 14B). Prior to transplantation, we observed that the 22-week-old human fetal testis contained a subset of cells positive for the germ-cell-specific protein VASA (FIG. 14Ci). Using whole-mount staining analyses, we immunostained with a primate-specific antibody that is known to recognize all human donor cells only (regardless of germ cell fate) and, consistent with reports on adult testis transplants, observed single-donor cells, small chains (FIG. 14Cii), or larger clusters of human donor cells on the basement membrane 2 months posttransplantation. In parallel, we also performed immunohistochemistry in cross-sections of testes transplanted with human fetal testis cells. In order to detect all human donor cells in recipient testes, we assessed expression of NuMA (FIG. 14D), a well-characterized protein that exclusively labels human cells and tissues. We observed significant human germ cell engraftment 8 weeks posttransplantation (FIG. 14Ciii).

Xenotransplantation of hESCs and Human iPSCs to Seminiferous Tubules Directs Germ Cell Development.

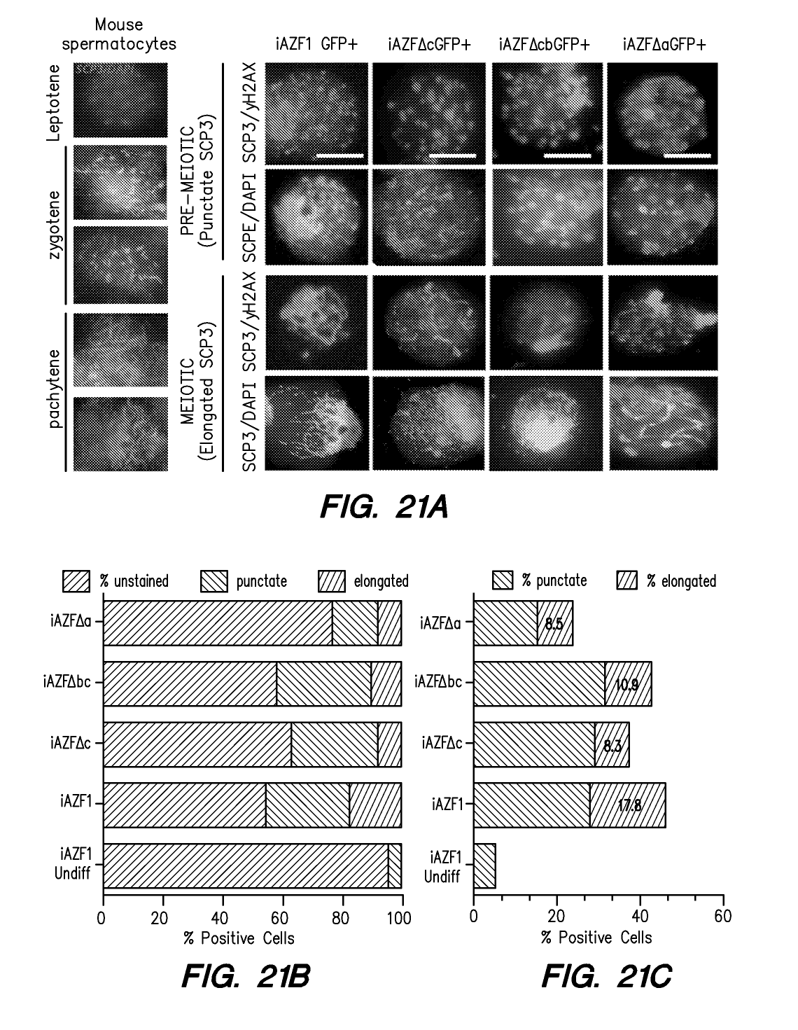

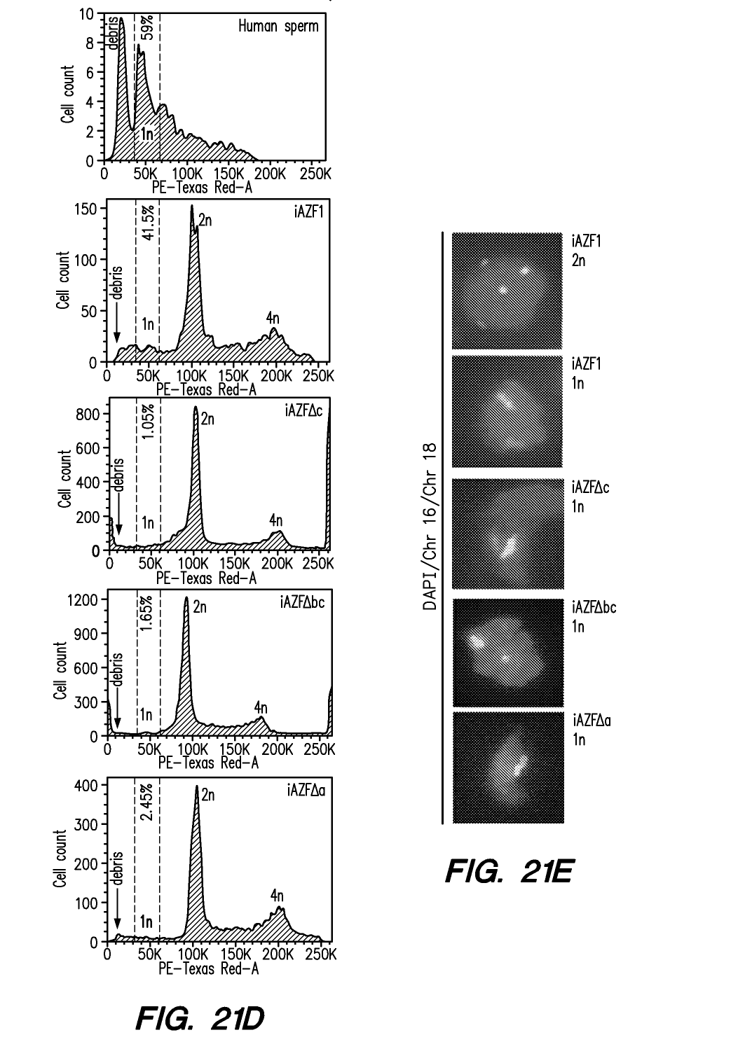

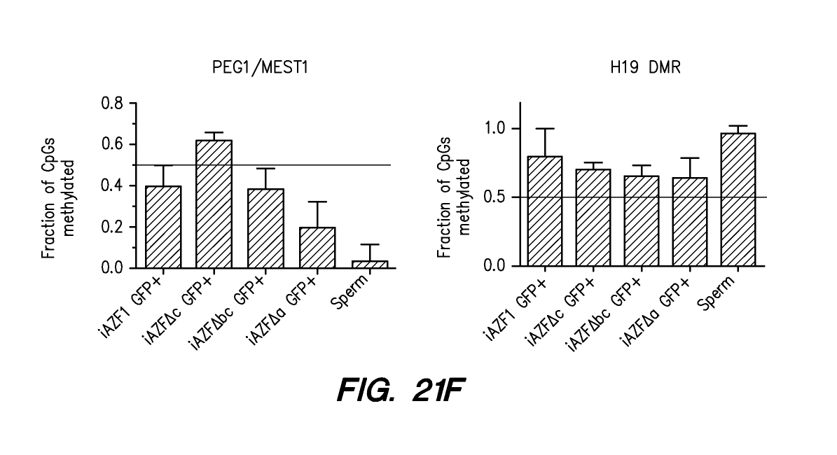

As noted above, we hypothesized that human iPSCs, which are distinct from mouse iPSCs (miPSCs), would survive, engraft, and be directed to a germ cell developmental fate if directly injected into the mouse seminiferous tubule, in response to instructive cues from the niche. Thus, we next transplanted undifferentiated male hESCs (H1) and the iAZF1 control iPSCs to eight recipient testes each and analyzed results by whole-mount immunohistochemistry as performed previously with human fetal testis transplants (FIG. 14A). We observed that all donor-derived cells were detected either as single cells or in clusters of cells; no clear evidence of chain-like structures was observed (FIG. 21B). We note that chain formation in human donor cells is a hallmark of spermatogonia but is not a property of PGCs and gonocytes. Thus, we reasoned that engrafted cells might have differentiated to PGCLCs or gonocyte-like cells and tested systematically for the presence of germ-cell-specific proteins on NuMA+ surviving donor cells using serial immunohistochemical analysis across testis cross-sections. We observed that all transplantations, regardless of fertility status, resulted in two fates of donor undifferentiated iPSCs in the testes: (1) differentiation to GCLCs that were spatially located near or at the basement membrane of seminiferous tubules and (2) extensive proliferation of iPSCs distant from the basement membrane most often within the interstitial space. These proliferative cells resembled embryonal carcinoma (EC) and yolk sac tumors (YSTs).

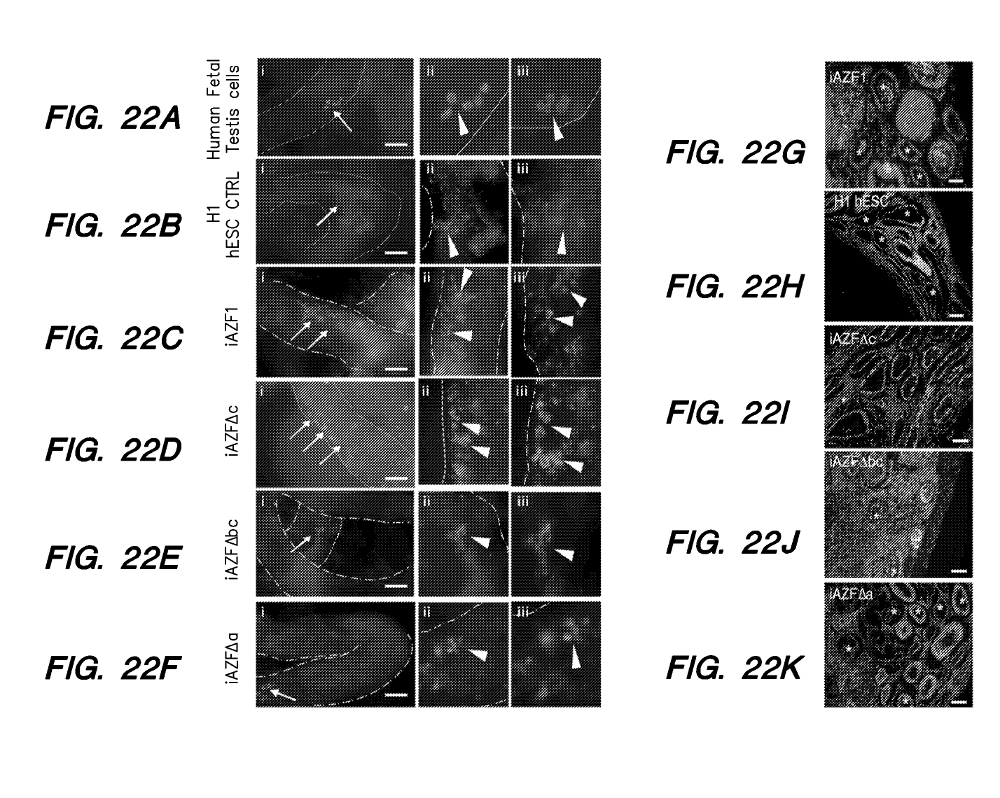

Upon further analysis of H1 and iAZF1 xenotransplants, and as expected, we observed that a subset of tubules was occupied with NuMA+ donor cells (FIGS. 21C-21I). In tubules containing donor cells, NuMA+ donor cells engrafted both near the basement membrane and in the center of the tubule. A significant number of NuMA+ cells coexpressed the germline protein, VASA (henceforth referred to as NuMA+/VASA+ cells) (FIG. 14E, white asterisks). We also observed that a few NuMA+ cells negative for VASA engrafted at the edges of seminiferous tubules (FIG. 14E, red asterisk). To exclude the possibility that NuMA+ donor cells could differentiate to Sertoli cells, we costained for GATA4 and SOX9, two nuclear proteins expressed by mouse and human Sertoli cells (FIG. 22). We did not observe the localization of SOX9 and GATA4 signals in nuclei of NuMA+ cells (FIGS. 22A and 22D), but only in cells that resemble human or mouse Sertoli cells at the edge of most tubules (FIGS. 22B, 22C, and 22E). Based on these results, we defined human GCLCs by the presence of NuMA+ cells that engrafted near the basement membrane of seminiferous tubules, colocalization of germ cell markers, especially VASA, and overall morphological resemblance to human fetal germ cells (FIG. 14E). By comparison, staining of human fetal testis cross-sections revealed VASA+ germ cells near the tubule basement membrane (FIG. 14Ci). Specifically, in testis xenografts, a large number of NuMA+/VASA+ cells localized to the basal membrane of the tubule either as single cells, rows or in clusters that bear clear resemblance to the arrangement of VASA+ germ cells of the human fetal testis (FIGS. 14E and 15A-4F).

Male Donor-Derived Cells Engrafted Outside Spermatogonial Tubules Remain Undifferentiated or Differentiate to Primitive Tumors.

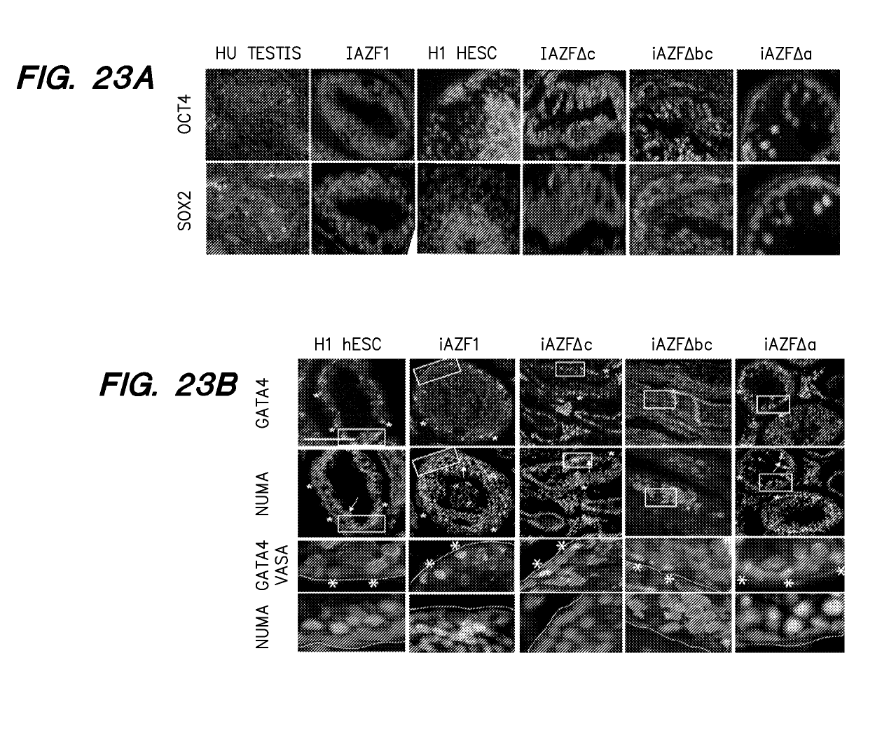

As noted above, in addition to cells that engrafted at the basement membrane, we observed that donor cells that filled the entire seminiferous tubule or exited the tubule proliferated extensively and did not demonstrate clear differentiation to either GCLCs or somatic cells. Instead, based on histology of xenografts, cells distant from the basement membrane, outside the tubules, resembled the histology of EC or, in some instances, YST cells (FIG. 23A). To verify, we stained xenografts for SOX2 and OCT4, protein markers diagnostic, at least in part, for EC formation and undifferentiated cells. We observed extensive SOX2 and OCT4 expression in NuMA+ nuclei in the interstitial space (FIG. 23B). We reasoned that these cells likely resulted from leakage of donor hESCs and iPSCs from tubules into the interstitial spaces of the testis. Notably, donor cells in the interstitial spaces did not form teratomas as assessed by thorough histological analysis, suggesting that they did not receive the appropriate cues to differentiate to somatic lineages or that somatic cells are efficiently removed following differentiation. NuMA+ cells that filled the entire tubular space expressed OCT4 but did not appear to form GCLCs (VASA negative), suggesting that they remained undifferentiated (FIG. 23C). We determined the efficiency of interstitial tumor formation across all testis xenografts performed and found that, except for human fetal testis transplantation, hESC and iPSC xenotransplants consistently produced interstitial tumors in more than half of all samples (FIG. 23D). Curiously, iAZFΔc iPSCs produced interstitial tumors only 30% of the time, but the tumors were always of EC or YST types. Altogether, these results suggested that the environment inside the seminiferous tubule is permissive for germ cell formation but appears to prevent differentiation of some donor cells to somatic lineages. In contrast, the environment outside the tubules promotes somatic differentiation but not teratoma formation in donor cells that exited tubules.

Transplantation of iPSCs into Murine Seminiferous Tubules Reveals Differences in Germ Cell Differentiation Between Control and AZF-Deleted Lines.

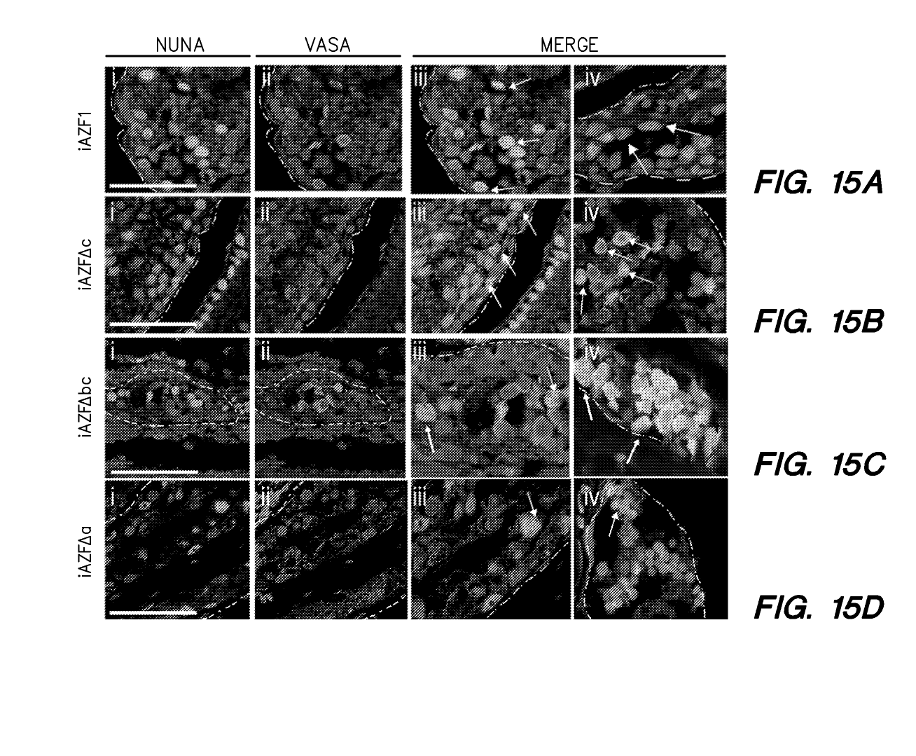

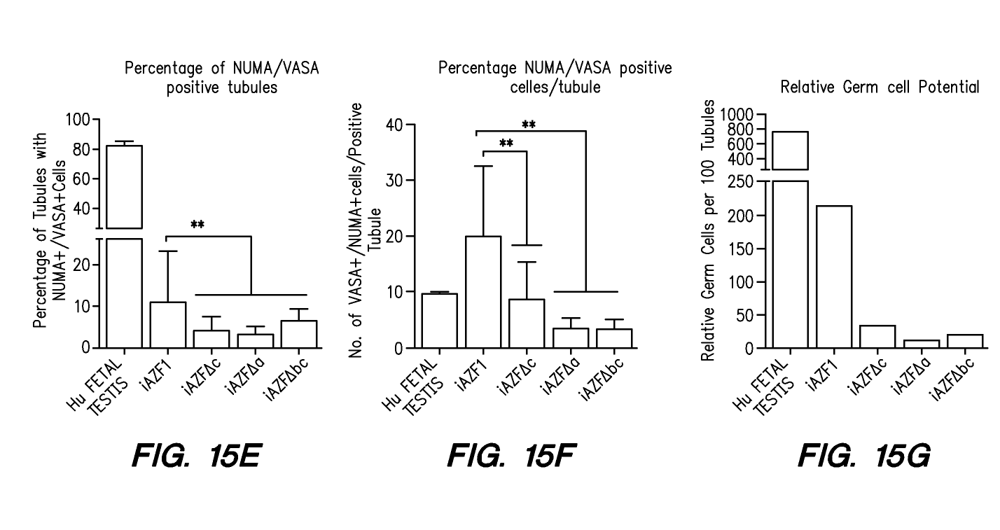

We predicted that AZF-deleted iPSCs would form and/or maintain fewer GCLCs than AZF-intact iPSC lines. From our immunohistochemical analysis, we observed that transplanted human fetal testis cells, H1 hESCs, iAZF1, iAZF2, and iAZFΔc iPSCs were usually localized in NuMA+/VASA+ clusters containing at least three or more cells (FIGS. 14E and 15A-4D, arrows and white asterisks). In contrast, iAZFΔbc and iAZFΔa iPSCs gave rise to significantly fewer clusters of NuMA+/VASA+ cells (FIGS. 15E and 15F, arrows and white asterisks). To quantify this observation for each donor sample, we counted NuMA+/VASA+ cells and tubules across entire cross-sections (20× magnification) at three to four different depths of cross-sections and in at least four biological replicates per sample. Using this strategy, we extracted the average percentage of positive seminiferous tubules (FIG. 15G). Although human fetal donor cells always produced the highest percentage of positive tubules (>30%), both iAZF1 and iAZF2 lines produced significantly higher tubule occupancy over AZF-deleted iPSCs (FIG. 15G). We next determined the average number of NuMA+/VASA+ cells in each positively stained tubule (FIG. 15H). We determined that, on average, >30 NuMA+/VASA+ cells per tubule were observed with human fetal donor cells and approximately 20-25 NuMA+/VASA+ cells per tubule with human iAZF1 and iAZF2 donor cells. These values were significantly higher than those observed in the case of iAZFΔa, iAZFΔbc, and iAZFΔc donor cells, where we observed four to eight NuMA+/VASA+ cells per tubule (FIG. 15H). To determine the relative germ-cell-forming potential of each donor cell population, we multiplied the tubule occupancy (per 100 tubules) by NuMA+/VASA+ cells per tubule (FIG. 15I). Our calculations reveal an approximate 50- to 100-fold reduction in formation of GCLCs from AZF-deleted iPSCs relative to AZF-intact iPSCs.

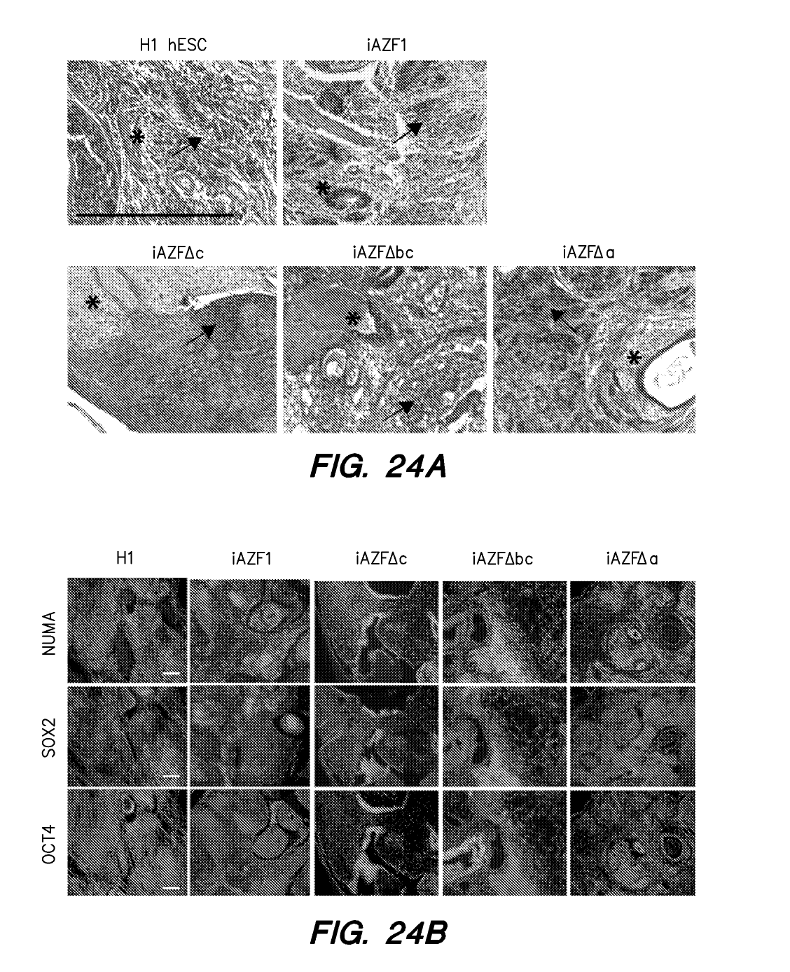

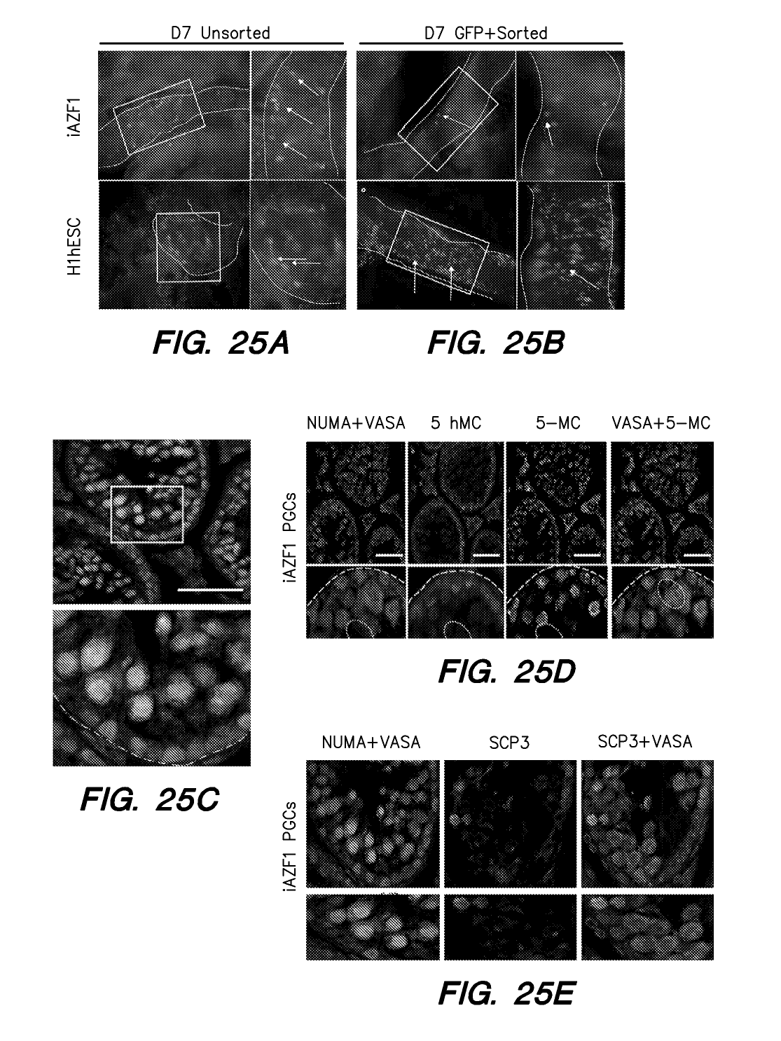

To further validate in vivo GCLC formation from donor cells, we examined expression of the germ-cell-specific proteins DAZL, PLZF, UTF1, STELLA, and DAZ in donor-derived GCLCs from xenotransplants (FIGS. 15 and 24). Based on previous reports, we predicted that STELLA, DAZL, and VASA would label iPSCs that had differentiated to PGCLCs, whereas UTF1, PLZF, and DAZ proteins would label iPSCs that had entered the pool of gonocyte-like or prospermatogonia-like cells and would overlap with expression of VASA. Fetal testes at 22 weeks of gestation are expected to contain gonocytes and undifferentiated spermatogonia. Interestingly, fetal germ cells expressed DAZL, STELLA, and UTF1 simultaneously (FIG. 24A). NuMA+/VASA+ cells derived from human fetal testis donor cells expressed the PGC proteins STELLA and DAZL in a similar nuclear and cytoplasmic pattern, respectively, to endogenous germ cells in the human fetal testis (FIG. 16A, panel 1, and FIG. 24A). Similarly, NuMA+/VASA+ cells derived from all AZF-intact and AZF-deleted donor lines expressed STELLA and DAZL proteins (FIGS. 16B-16F, panel 1). We further observed that expression of UTF1, PLZF, and DAZ proteins varied between donor cell lines dependent on genotype. In the control human fetal testis, UTF1, PLZF, and DAZ proteins were expressed in a few GCLCs in a subset of seminiferous tubules (FIG. 24A). In a similar fashion to the fetal testis, UTF1 was localized in several DAZL+ donor-derived germ cells near the edges of tubules in all samples except iAZFΔbc and iAZFΔa, where we could not detect UTF1 signals (FIGS. 16A-5D, panel 2, and FIG. 24). In addition, the prospermatogonial protein PLZF was detected in only a handful of VASA+ cells in iAZF1 xenografts, but not in other samples (FIG. 16C, panel 2). We further observed that none of the three AZF-deleted lines expressed the Y chromosome-encoded protein DAZ. In contrast, we detected cytoplasmic expression of DAZ proteins in NuMA+ cells of AZF-intact donor cells (H1, iAZF1, and iAZF2) and donor cells from the human fetal testis (FIGS. 16G and 24B). The pattern of DAZ expression closely corresponded to that of endogenous fetal germ cells in the human testis (FIG. 6A). Collectively, results indicate that the human fetal testis donor cells and all patient-derived iPSCs are capable of forming PGCLCs but that, in general, AZF-deleted iPSCs form fewer GCLCs with altered expression of germ-cell-specific proteins (FIG. 16H).

Epigenetic Analysis of Donor-Derived GCLCs from AZF-Intact iPSCs.

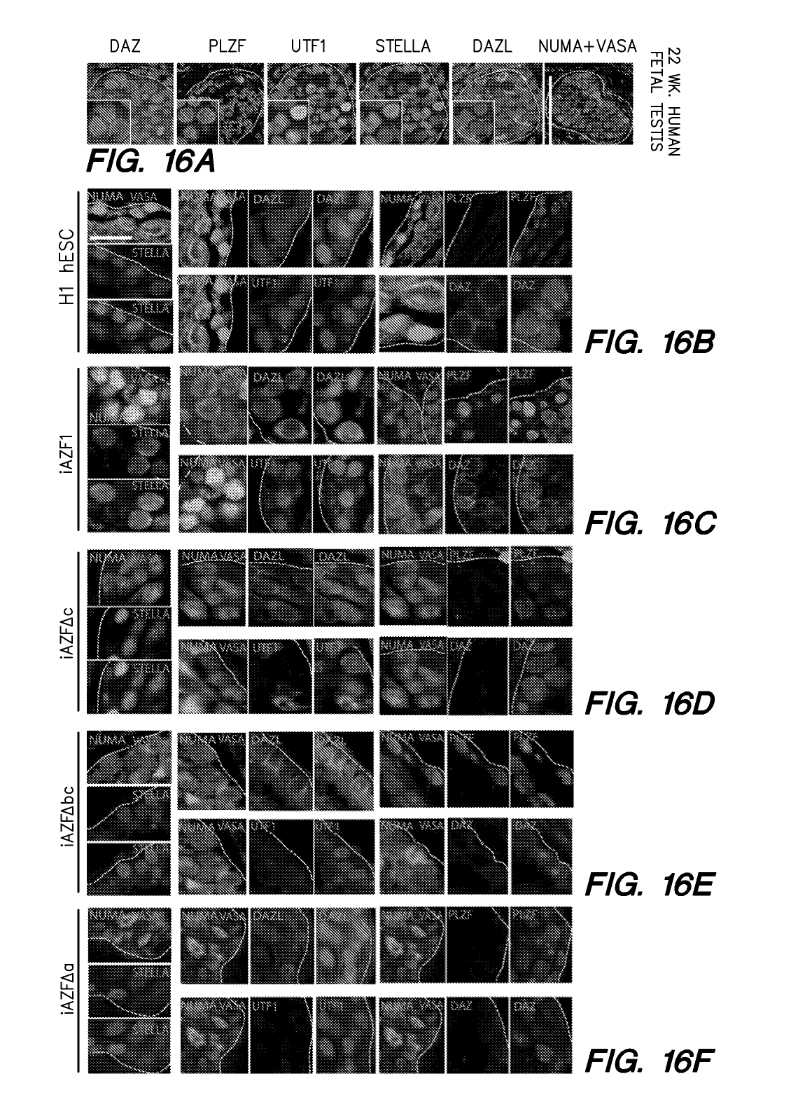

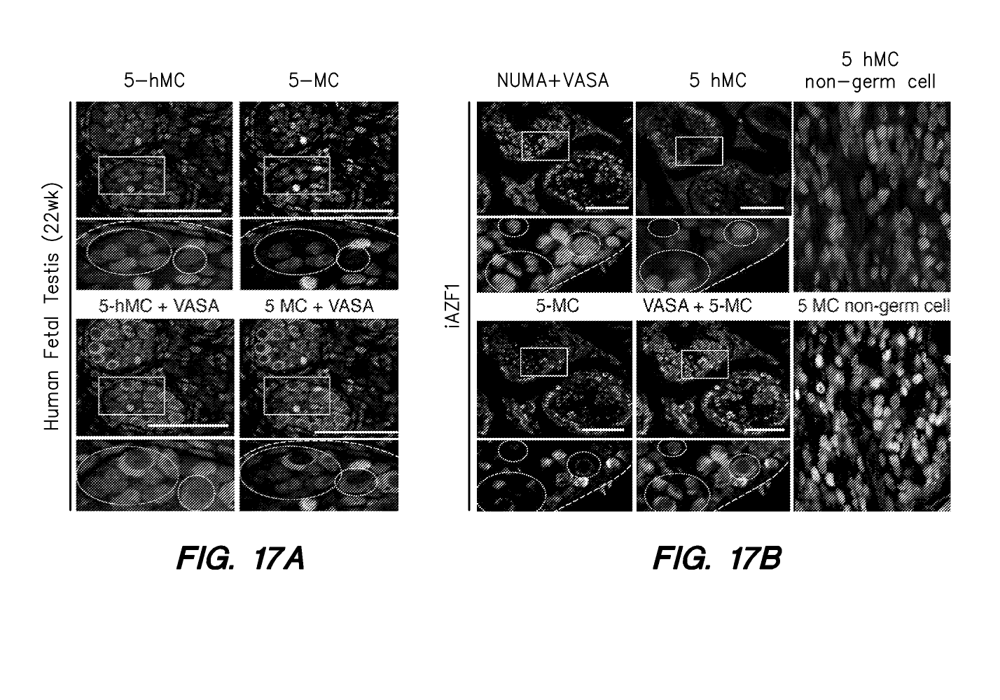

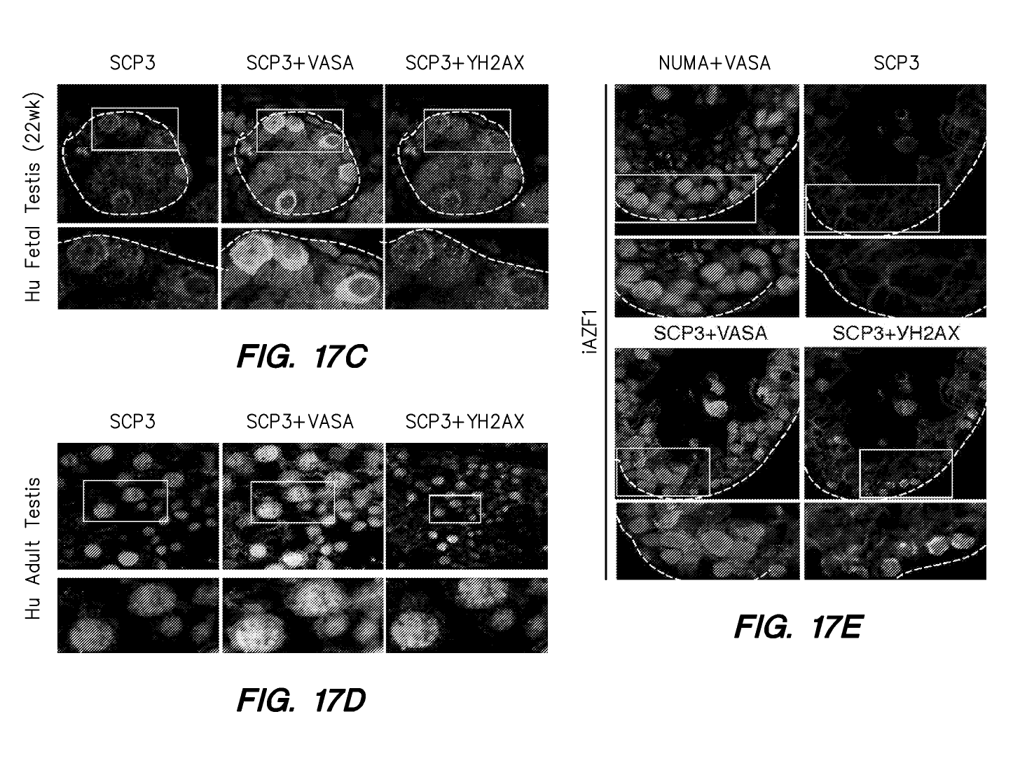

In order to evaluate if epigenetic reprogramming, characteristic of endogenous germ cells, occurs in donor iPSC xenografts, we performed immunohistochemistry for 5-methylcytosine (5-mC) as a marker of global CpG methylation. We compared 5-mC status in endogenous germ cells of both human fetal and adult testes (FIGS. 17A and 17B) and in all recipient mouse testis xenografts (FIGS. 17C-17G). We observed that a majority of endogenous VASA+ germ cells in fetal testes were 5-mC positive, with a subset of VASA+ cells 5-mC negative (FIG. 17A, yellow arrowheads and white dotted circles, respectively). Intriguingly, NuMA+/VASA+ cells in all xenografts derived from human fetal testis donor cells or iPSC lines exhibited similar numbers of cells negative or positive for 5-mC (FIGS. 17C-6G, yellow arrowheads and white dotted circles, respectively). The 5-mC reduction was confined to germ cells within tubules (i.e., NuMA+ donor cells outside tubules were all 5-mC positive) (FIG. 17H). Additionally, undifferentiated iPSC donor cells in culture exhibited uniform levels of 5-mC prior to transplantation (FIG. 17H, bottom). In addition to immunohistochemical analysis, we quantified the pattern of 5-mC signals in NuMA+/VASA+ cells within tubules and observed a fairly uniform percentage (15%-20%) of 5-mC-negative cells in all xenografts, including those derived from human fetal testis donor cells. This observation mirrored the approximate 25% of endogenous fetal testis germ cells that were 5-mC negative (FIG. 17I). Meanwhile, endogenous adult testis germ cells were highly methylated (only 3%-5% 5-mC negative). Collectively, our results demonstrate that human donor cells undergo global DNA demethylation upon initiation of germ cell differentiation inside the mouse seminiferous tubule.

A major emphasis in stem cell biology and regenerative medicine is focused on iPSC-derived cell transplantation to restore somatic cellular and tissue function. Much less focus has been directed at the use of human iPSCs to derive germ cells for potential cell replacement therapies, despite elegant studies in the mouse that demonstrate the ability to completely reconstitute mouse germline development from ESCs and iPSCs. Both mouse and human studies have demonstrated requirements for key germ-cell-specific genes and dependence of germ cell development on interaction with the niche. Yet, there are difficulties in interpreting the human data given the low numbers of GCLCs formed in vitro, the extensive overlap of gene expression shared between ESCs and PGCs such that fewer than a dozen genes may differ, and the asynchrony in development of germ cells in vitro.

In this study, we observed that transplantation of normal and AZF-deleted iPSCs into the environment of the seminiferous tubule resulted in GCLC formation, whereas iPSCs outside the tubules failed to differentiate to GCLCs. Based on these results, we reason that iPSC and hESC donor cells inside seminiferous tubules may receive signals that permit them to engraft near the basement membrane. Our findings are supported by the observation that human spermatogonia transplanted to mouse testes “home” to the basement membrane but cannot differentiate to more mature stages. We show that cell-to-cell contacts between cells of the basement membrane niche, especially Sertoli cells, and donor cells enable exchange of instructive signals. Furthermore, this crosstalk favors acquisition of a PGC- or gonocyte-like fate in human iPSCs with suppression of extensive proliferation and somatic cell differentiation. In a similar fashion, undifferentiated murine spermatogonia self-renew or differentiate based on Sertoli cell density and factors secreted by Sertoli cells. These Sertoli cell-produced factors include paracrine growth factors such as transforming growth factor (TGF) and stem cell factor (SCF), which have been implicated in the survival and/or differentiation of PGCs. In mice, the transition to epiblast stem cells and overexpression of Prdm14 facilitates germ cell differentiation posttransplantation. In contrast, both undifferentiated hESCs and human iPSCs are believed to be “epiblast-like” and moreover express high levels of PRDM14 endogenously as shown herein.

Based on our results, undifferentiated human iPSCs inside murine seminiferous tubules receive critical molecular cues from Sertoli cells and, potentially, from peri-tubular cells that assist in localization and physical interaction, accessibility to signaling cues, and ultimately direct germline differentiation. Moreover, if the niche is “overbooked” and iPSCs leak out of tubules or are not able to contact Sertoli cells, these cells will proliferate extensively to form EC outside the tubule or remain undifferentiated inside the tubule. Indeed, it is remarkable that in sharp contrast to studies with miPSCs, human iPSCs and hESCs never formed teratomas in our studies, perhaps due to their transient interaction with the intratubular environment.

Historically, it has not been possible to address when or how germ cells are depleted in men with spontaneous deletions; heterogeneity of clinical phenotypes associated with the same genetic deletions may be linked to depletion rate or time of presentation. Moreover, it was not known whether AZF deletions might disrupt early germ cell development or maintenance. Here, we observed that iPSCs derived from men with AZFa and AZFbc deletions formed fewer and poor-quality PGCLCs both in vitro and in vivo. Regardless of genotype, however, we determined that human donor-derived GCLCs are induced to undergo global DNA demethylation from somatic methylation levels in a manner that is characteristic of the development of male PGCs to gonocytes. Importantly, our results indicate that there is a clear defect linked to expression of later genes such as DAZ, PLZF, and UTF1 that coexpress with or follow expression of PGC markers; in particular, those with AZF deletions did not express Y chromosome DAZ and the genes UTF1 and PLZF in any germ cells. Thus, our results of germ cell differentiation are well correlated with clinical outcomes but also provide some interesting insights. iPSCs formed PGCLCs, suggesting that men with these deletions likely form germ cells early in development but that these cells are depleted prior to clinical presentation. This concept is consistent with many mouse models of infertility in which initial populations of germ cells are not maintained. Human genetic data indicate that the XO genotype (Turner syndrome) is compatible with formation of fetal germ cells, but they are depleted by birth or thereafter.

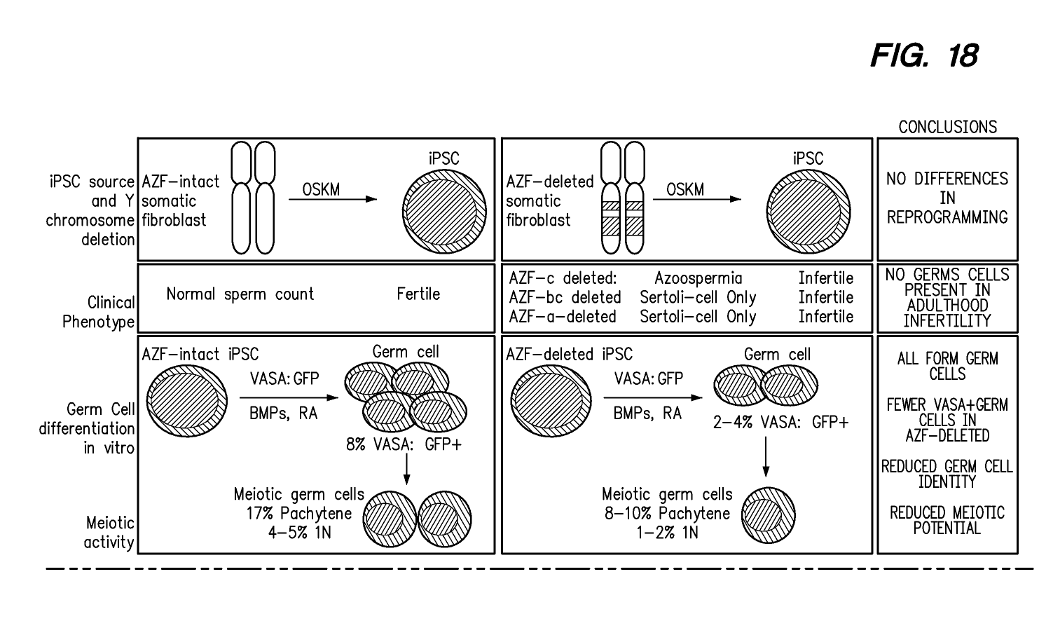

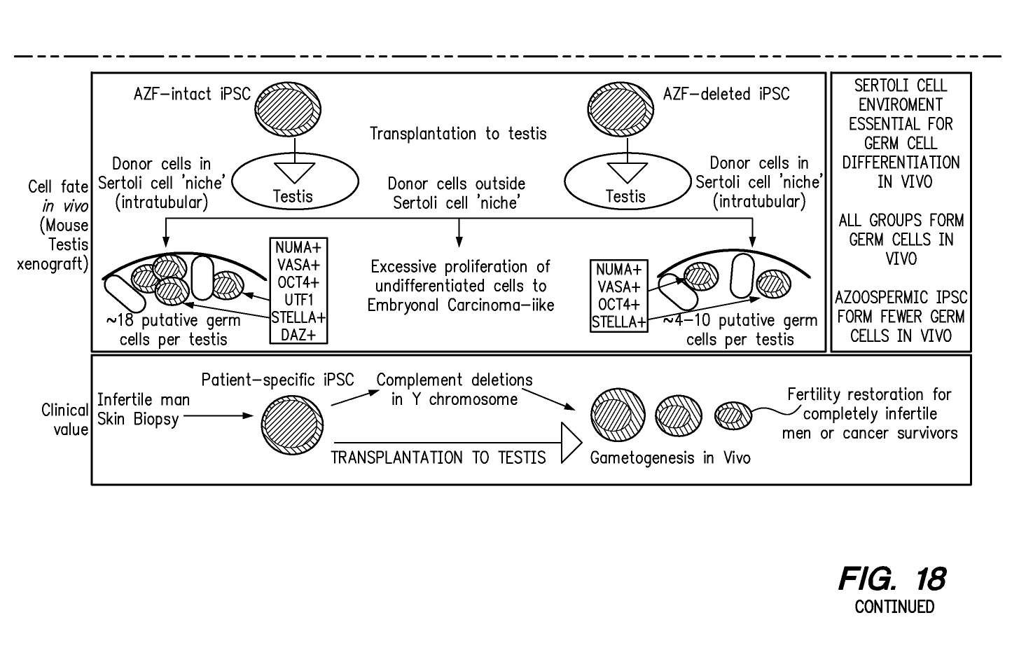

Our results have been summarized into a working model for the efficiency and fate of human iPSCs during human germ cell formation in the mouse testes (FIG. 18).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for the treatment of male infertility, the method comprising:
obtaining a somatic cell from a male mammal suffering from infertility;
reprogramming the somatic cell to pluripotency by contacting with an effective dose of a cocktail of reprogramming factors to generate a population of pluripotent cells;
transplanting the population of pluripotent cells directly into the seminiferous tubules of the male, wherein the pluripotent cells undergo spermatogenesis.

2. The method of claim 1, wherein the male is a human.

3. The method of claim 2, wherein the infertility is caused by a deletion in the AZF region of the Y chromosome.

4. The method of claim 1, wherein the reprogramming factors provide for integration-free reprogramming.

5. The method of claim 1, wherein the cocktail of reprogramming factors comprises a member of the Oct family, a member of the Sox family, a member of the Kif family, and a member of the Myc family.

6. The method of claim 5, wherein the cocktail of reprogramming factors comprises Oct3/4; Sox2; Klf4; and c-Myc (OSKM).

7. The method of claim 6, wherein, during reprogramming, the cells are also contacted with an effective dose of VASA (OSKMV) as a protein or a nucleic acid encoding the protein.

8. The method of claim 5, wherein the factors are provided as modified mRNA.

9. The method of claim 8, wherein cells are transfected with the modified mRNA daily for a period of from about 10 to about 20 days.

10. The method of claim 9, wherein the mRNA is provided at a concentration of from 0.06 to about 0.12 ng/cell.

11. The method of claim 8, wherein the cocktail is OSKMV, and the ratio of factors is 3:0.5:1:0.5:1.

12. The method of claim 11, wherein the somatic cells are repeatedly transfected with the modified mRNA.

13. A method of generating male germ cells, the method comprising:
introducing pluripotent stem cells into a Sertoli cell environment, wherein the pluripotent cells are induced to differentiate into male germ cells.

14. The method of claim 13, wherein the pluripotent stem cells are reprogrammed from somatic cells.

15. The method of claim 14, wherein the Sertoli cell environment is in vivo.

16. The method of claim 15, wherein the pluripotent cells are reprogrammed with a cocktail of factors in combination with an effective dose of VASA.
